(19) United States
(12) Patent Application Publication
(10) Pub. No.: US 2022/0324924 A1
(43) Pub. Date: Oct. 13, 2022

(54) MULTISPECIFIC TRANSTHYRETIN IMMUNOGLOBULIN FUSIONS

(71) Applicant: AMGEN INC., Thousand Oaks, CA (US)

(72) Inventor: Kenneth William WALKER, Newbury Park, CA (US)

(73) Assignee: AMGEN INC., Thousand Oaks, CA (US)

(21) Appl. No.: 17/624,824

(22) PCT Filed: Jul. 6, 2020
(86) PCT No.: PCT/US2020/040873

§ 371 Date: Jan. 4, 2022

Related U.S. Application Data
(60) Provisional application No. 62/871,247, filed on Jul. 8, 2019.

(51) Int. Cl. C07K 14/47; C12N 5/071; C12N 15/85; C12N 15/62; C12P 21/02

(52) U.S. Cl. CPC C07K 14/47; C12N 5/0686; C12N 15/85; C12N 15/625; C12P 21/02; A61K 38/00

(57) ABSTRACT

The present invention relates to multispecific transthyretin (TTR) complexes useful as multispecific binding proteins. The multispecific TTR complexes described herein are particularly useful in binding to one, two, or more epitopes which may be present on one or more proteins. Methods for treating diseases using the TTR complexes of the present invention are described herein.

### CROSS REFERENCE TO RELATED APPLICATIONS

This application claims priority to and the benefit of U.S. Provisional Application No. 62/871,247 filed Jul. 8, 2019; which is incorporated by reference herein in its entirety.

### REFERENCE TO THE SEQUENCE LISTING

This application contains a Sequence Listing in computer-readable form. The Sequence Listing is provided as a text file entitled A-2414-WO-PCT_SeqList_ST25.txt, created Jul. 1, 2020, which is 101,660 bytes in size. The information in the electronic format of the Sequence Listing is incorporated herein by reference in its entirety.

### FIELD OF THE INVENTION

The present invention relates to multispecific transthyretin (TTR) complexes useful as multispecific binding proteins. The multispecific TTR complexes described herein are particularly useful in binding to one, two, or more epitopes which may be present on one or more proteins. Methods for treating diseases using the TTR complexes of the present invention are described herein.

### BACKGROUND OF THE INVENTION

Monospecific antibodies, i.e., antibodies that bind a single antigen, are a well-established class of compounds approved in a variety of therapeutic areas. Indeed, in the past decade, many monospecific antibody-based pharmaceuticals have been approved in various countries.

Multispecific proteins, such as multispecific antibodies, have been the subject of increasing amounts of research. Multispecific proteins are capable of binding to two or more different antigens on the same or different protein. This allows for the possibility or effecting two different biological pathways at the same time.

Transthyretin (TTR) is a non-covalent tetrameric human serum and cerebral spinal fluid protein that plays a role in carrying a portion of circulating thyroxine and in the serum half-life of retinol binding protein. TTR is typically present as a tetrameric (˜56 kDa) serum protein with each monomeric unit having an approximate molecular weight of 14 kDa.

Previous efforts to multimerize proteins include the use of streptavidin (Kipriyanov et al., Protein Engineering, 9(2):203-211 (1996)), helix-turn-helix constructs (Kriangkum et al., Biomolecular Engineering, 18:31-40 (2001)), leucine zippers (Kruif et al., The Journal of Biological Chemistry, 271(13):7630-7634, 1996 (1996)), barnase/barstar complexes (Deyev et al., Nature Biotechnology, 21(12):1486-1492 (2003)), and Dock N Lock technology (protein kinase and A-kinase anchoring protein anchoring domain interactions) (Goldenberg et al., Journal of Nuclear Medicine, 49(1):158-163 (2008)).

However, there remains a need for an efficient means of generating multispecific proteins (e.g., whole antibodies and antibody fragments) that can bind multiple epitopes on the same or different protein.

### SUMMARY OF THE INVENTION

The present invention relates to a TTR protein complex, wherein
the TTR protein complex comprises TTR subunits A, B, C, and D;
TTR subunits A and B dimerize to form TTR dimer AB;
TTR subunits C and D dimerize to form TTR dimer CD;
TTR dimers AB and CD further dimerize to form TTR tetramer ABCD; and
each of A, B, C, and D comprise the amino acid sequence of SEQ ID NO: 1, except that at least one amino acid in the interface between TTR dimer AB and TTR dimer CD is mutated such that the formation of an ABCD tetramer is favored over the formation of any other tetramer (e.g., an ABAB tetramer or a CDCD tetramer).

Each of the A, B, C, and D subunits of the TTR protein complex may comprise the amino acid sequence of SEQ ID NO: 1 with the following mutations: C10A, K15A, or both C10A and K15A.

Thus, in one embodiment, the present invention relates to TTR protein complexes wherein both A and B, both C and D, or all four of A, B, C, and D comprise a mutation at one or more amino acids positions selected from the list comprising: 6, 7, 8, 9, 10, 13, 15, 17, 19, 20, 21, 22, 23, 24, 26, 50, 51, 52, 53, 54, 56, 57, 60, 61, 62, 63, 78, 82, 83, 84, 85, 100, 101, 102, 103, 104, 106, 108, 110, 112, 113, 114, 115, 117, 119, 121, 123, 124, 125, 126, and 127 of SEQ ID NO: 1.

In another embodiment, the present invention relates to a TTR protein complex wherein both A and B, both C and D, or all four of A, B, C, and D comprise a mutation at one or more amino acids positions selected from the list comprising: 15, 17, 20, 21, 22, 23, 24, 51, 52, 84, 106, 108, 112, 114, 115, 119, 121, and 123 of SEQ ID NO: 1.

In another embodiment, the present invention relates to a TTR protein complex wherein both A and B, both C and D, or all four of A, B, C, and D comprise a mutation at one or more amino acids positions selected from the list comprising: 15, 17, 20, 21, 22, 23, 24, 51, 52, 84, 106, 108, 112, 114, 115, 119, 121, and 123 of SEQ ID NO: 1, wherein said amino acid is mutated to an aspartate, glutamate, arginine, lysine, or histidine.

In another embodiment, the present invention relates to a TTR protein complex wherein A and B comprise a mutation at one or more amino acids positions selected from the list comprising: 15, 17, 20, 21, 22, 23, 24, 51, 52, 84, 106, 108, 112, 114, 115, 119, 121, and 123 of SEQ ID NO: 1, wherein said amino acid is mutated to an aspartate or glutamate.

In yet another embodiment, the present invention relates to a TTR protein complex wherein C and D comprise a mutation at one or more amino acids positions selected from the list comprising: 15, 17, 20, 21, 22, 23, 24, 51, 52, 84, 106, 108, 112, 114, 115, 119, 121, and 123 of SEQ ID NO: 1, wherein said amino acid is mutated to an arginine, lysine, or histidine.

In a particular embodiment, A and B comprise a mutation at one or more amino acids positions selected from the list comprising: 15, 17, 20, 21, 22, 23, 24, 51, 52, 84, 106, 108, 112, 114, 115, 119, 121, and 123 of SEQ ID NO: 1, wherein said amino acid is mutated to an aspartate or glutamate; and C and D comprise a mutation at one or more amino acids positions selected from the list comprising: 15, 17, 20, 21, 22, 23, 24, 51, 52, 84, 106, 108, 112, 114, 115, 119, 121, and 123 of SEQ ID NO: 1, wherein said amino acid is mutated to an arginine, lysine, or histidine.

In some embodiments, A and B comprise at least one mutation in SEQ ID NO: 1, wherein said mutation is selected from the list comprising: K15D, L17D, V20D, R21D, G22D, S23D, P24D, S52D, I84D, T106D, A108D, S112D, Y114D, S115D, T119D, V121D, S123D, K15E, L17E, V20E, R21E, G22E, S23E, P24E, D51E, S52E, 184E, T106E, A108E, S112E, Y114E, S115E, T119E, V121E, and S123E. The present invention also relates to TTR protein complexes wherein A and B comprise at least one mutation in SEQ ID NO: 1, wherein the mutation is selected from the list comprising: L17D, L17E, V20D, V20E, G22D, G22E, S112D, S112E, T119D, T119E, V121D, and V121E.

In some embodiments, C and D comprise at least one mutation in SEQ ID NO: 1, wherein said mutation is selected from the list comprising: K15R, L17R, V20R, G22R, S23R, P24R, D51R, S52R, 184R, T106R, A108R, S112R, Y114R, S115R, T119R, V121R, S123R, L17K, V20K, R21K, G22K, S23K, P24K, D51K, S52K, 184K, T106K, A108K, S112K, Y114K, S115K, T119K, V121K, S123K, K15H, L17H, V20H, R21H, G22H, S23H, P24H, D51H, S52H, I84H, T106H, A108H, S112H, Y114H, S115H, T119H, V121H, and S123H. The present invention also relates to TTR protein complexes wherein C and D comprise at least one mutation in SEQ ID NO: 1, wherein the mutation is selected from the list comprising: L17R, L17K, L17H, V20R, V20K, V20H, G22R, G22K, G22H, S112R, S112K, S112H, T119R, T119K, T119H, V121R, V121K, and V121H.

In other embodiments, both A and B, both C and D, or all four of A, B, C, and D independently comprise one mutation discussed above. In yet other embodiments, both A and B, both C and D, or all four of A, B, C, and D independently comprise two said mutations discussed above.

In specific embodiments, the present invention relates to a TTR protein complex wherein each of A, B, C, and D comprise the amino acid sequence of SEQ ID NO: 1 with the following mutations:

A and B comprise C10A/K15A/L17D, and C and D comprise C10A/K15A/L17R (or vice versa);
A and B comprise C10A/K15A/L17E, and C and D comprise C10A/K15A/L17R (or vice versa);
A and B comprise C10A/K15A/V20D, and C and D comprise C10A/K15A/L17R (or vice versa);
A and B comprise C10A/K15A/V20E, and C and D comprise C10A/K15A/L17R (or vice versa);
A and B comprise C10A/K15A/G22D, and C and D comprise C10A/K15A/L17R (or vice versa);
A and B comprise C10A/K15A/G22E, and C and D comprise C10A/K15A/L17R (or vice versa);
A and B comprise C10A/K15A/S112D, and C and D comprise C10A/K15A/L17R (or vice versa);
A and B comprise C10A/K15A/S112E, and C and D comprise C10A/K15A/L17R (or vice versa);
A and B comprise C10A/K15A/T119D, and C and D comprise C10A/K15A/L17R (or vice versa);
A and B comprise C10A/K15A/T119E, and C and D comprise C10A/K15A/L17R (or vice versa);
A and B comprise C10A/K15A/V121D, and C and D comprise C10A/K15A/L17R (or vice versa);
A and B comprise C10A/K15A/V121E, and C and D comprise C10A/K15A/L17R (or vice versa);
A and B comprise C10A/K15A/L17D, and C and D comprise C10A/K15A/L17K (or vice versa);
A and B comprise C10A/K15A/L17E, and C and D comprise C10A/K15A/L17K (or vice versa);
A and B comprise C10A/K15A/V20D, and C and D comprise C10A/K15A/L17K (or vice versa);
A and B comprise C10A/K15A/V20E, and C and D comprise C10A/K15A/L17K (or vice versa);
A and B comprise C10A/K15A/G22D, and C and D comprise C10A/K15A/L17K (or vice versa);
A and B comprise C10A/K15A/G22E, and C and D comprise C10A/K15A/L17K (or vice versa);
A and B comprise C10A/K15A/S112D, and C and D comprise C10A/K15A/L17K (or vice versa);
A and B comprise C10A/K15A/S112E, and C and D comprise C10A/K15A/L17K (or vice versa);
A and B comprise C10A/K15A/T119D, and C and D comprise C10A/K15A/L17K (or vice versa);
A and B comprise C10A/K15A/T119E, and C and D comprise C10A/K15A/L17K (or vice versa);
A and B comprise C10A/K15A/V121D, and C and D comprise C10A/K15A/L17K (or vice versa);
A and B comprise C10A/K15A/V121E, and C and D comprise C10A/K15A/L17K (or vice versa);
A and B comprise C10A/K15A/L17D, and C and D comprise C10A/K15A/V20R (or vice versa);
A and B comprise C10A/K15A/L17E, and C and D comprise C10A/K15A/V20R (or vice versa);
A and B comprise C10A/K15A/V20D, and C and D comprise C10A/K15A/V20R (or vice versa);
A and B comprise C10A/K15A/V20E, and C and D comprise C10A/K15A/V20R (or vice versa);
A and B comprise C10A/K15A/G22D, and C and D comprise C10A/K15A/V20R (or vice versa);
A and B comprise C10A/K15A/G22E, and C and D comprise C10A/K15A/V20R (or vice versa);
A and B comprise C10A/K15A/S112D, and C and D comprise C10A/K15A/V20R (or vice versa);
A and B comprise C10A/K15A/S112E, and C and D comprise C10A/K15A/V20R (or vice versa);
A and B comprise C10A/K15A/T119D, and C and D comprise C10A/K15A/V20R (or vice versa); A and B comprise C10A/K15A/T119E, and C and D comprise C10A/K15A/V20R (or vice versa);
A and B comprise C10A/K15A/V121D, and C and D comprise C10A/K15A/V20R (or vice versa);
A and B comprise C10A/K15A/V121E, and C and D comprise C10A/K15A/V20R (or vice versa);
A and B comprise C10A/K15A/L17D, and C and D comprise C10A/K15A/V20K (or vice versa);
A and B comprise C10A/K15A/L17E, and C and D comprise C10A/K15A/V20K (or vice versa);
A and B comprise C10A/K15A/V20D, and C and D comprise C10A/K15A/V20K (or vice versa);
A and B comprise C10A/K15A/V20E, and C and D comprise C10A/K15A/V20K (or vice versa);
A and B comprise C10A/K15A/G22D, and C and D comprise C10A/K15A/V20K (or vice versa);
A and B comprise C10A/K15A/G22E, and C and D comprise C10A/K15A/V20K (or vice versa);
A and B comprise C10A/K15A/S112D, and C and D comprise C10A/K15A/V20K (or vice versa);
A and B comprise C10A/K15A/S112E, and C and D comprise C10A/K15A/V20K (or vice versa);
A and B comprise C10A/K15A/T119D, and C and D comprise C10A/K15A/V20K (or vice versa);
A and B comprise C10A/K15A/T119E, and C and D comprise C10A/K15A/V20K (or vice versa);
A and B comprise C10A/K15A/V121D, and C and D comprise C10A/K15A/V20K (or vice versa);
A and B comprise C10A/K15A/V121E, and C and D comprise C10A/K15A/V20K (or vice versa);
A and B comprise C10A/K15A/L17D, and C and D comprise C10A/K15A/G22R (or vice versa);
A and B comprise C10A/K15A/L17E, and C and D comprise C10A/K15A/G22R (or vice versa);
A and B comprise C10A/K15A/V20D, and C and D comprise C10A/K15A/G22R (or vice versa);
A and B comprise C10A/K15A/V20E, and C and D comprise C10A/K15A/G22R (or vice versa);
A and B comprise C10A/K15A/G22D, and C and D comprise C10A/K15A/G22R (or vice versa);
A and B comprise C10A/K15A/G22E, and C and D comprise C10A/K15A/G22R (or vice versa);
A and B comprise C10A/K15A/S112D, and C and D comprise C10A/K15A/G22R (or vice versa);
A and B comprise C10A/K15A/S112E, and C and D comprise C10A/K15A/G22R (or vice versa);
A and B comprise C10A/K15A/T119D, and C and D comprise C10A/K15A/G22R (or vice versa);
A and B comprise C10A/K15A/T119E, and C and D comprise C10A/K15A/G22R (or vice versa);
A and B comprise C10A/K15A/V121D, and C and D comprise C10A/K15A/G22R (or vice versa);
A and B comprise C10A/K15A/V121E, and C and D comprise C10A/K15A/G22R (or vice versa);
A and B comprise C10A/K15A/L17D, and C and D comprise C10A/K15A/G22K (or vice versa);
A and B comprise C10A/K15A/L17E, and C and D comprise C10A/K15A/G22K (or vice versa);
A and B comprise C10A/K15A/V20D, and C and D comprise C10A/K15A/G22K (or vice versa);
A and B comprise C10A/K15A/V20E, and C and D comprise C10A/K15A/G22K (or vice versa);
A and B comprise C10A/K15A/G22D, and C and D comprise C10A/K15A/G22K (or vice versa);
A and B comprise C10A/K15A/G22E, and C and D comprise C10A/K15A/G22K (or vice versa);
A and B comprise C10A/K15A/S112D, and C and D comprise C10A/K15A/G22K (or vice versa);
A and B comprise C10A/K15A/S112E, and C and D comprise C10A/K15A/G22K (or vice versa); A and B comprise C10A/K15A/T119D, and C and D comprise C1 OA/K15A/G22K (or vice versa);
A and B comprise C10A/K15A/T119E, and C and D comprise C10A/K15A/G22K (or vice versa);
A and B comprise C10A/K15A/V121D, and C and D comprise C10A/K15A/G22K (or vice versa);
A and B comprise C10A/K15A/V121E, and C and D comprise C10A/K15A/G22K (or vice versa);
A and B comprise C10A/K15A/L17D, and C and D comprise C10A/K15A/S112R (or vice versa);
A and B comprise C10A/K15A/L17E, and C and D comprise C10A/K15A/S112R (or vice versa);
A and B comprise C10A/K15A/V20D, and C and D comprise C10A/K15A/S112R (or vice versa);
A and B comprise C10A/K15A/V20E, and C and D comprise C10A/K15A/S112R (or vice versa);
A and B comprise C10A/K15A/G22D, and C and D comprise C10A/K15A/S112R (or vice versa);
A and B comprise C10A/K15A/G22E, and C and D comprise C10A/K15A/S112R (or vice versa);
A and B comprise C10A/K15A/S112D, and C and D comprise C10A/K15A/S112R (or vice versa);
A and B comprise C10A/K15A/S112E, and C and D comprise C10A/K15A/S I12R (or vice versa);
A and B comprise C10A/K15A/T119D, and C and D comprise C10A/K15A/S112R (or vice versa);
A and B comprise C10A/K15A/T119E, and C and D comprise C10A/K15A/S112R (or vice versa);
A and B comprise C10A/K15A/V121D, and C and D comprise C10A/K15A/S112R (or vice versa);
A and B comprise C10A/K15A/V121E, and C and D comprise C1 OA/K15A/S112R (or vice versa);
A and B comprise C10A/K15A/L17D, and C and D comprise C10A/K15A/S112K (or vice versa);
A and B comprise C10A/K15A/L17E, and C and D comprise C10A/K15A/S112K (or vice versa);
A and B comprise C10A/K15A/V20D, and C and D comprise C10A/K15A/S112K (or vice versa);
A and B comprise C10A/K15A/V20E, and C and D comprise C10A/K15A/S112K (or vice versa);
A and B comprise C10A/K15A/G22D, and C and D comprise C10A/K15A/S112K (or vice versa);
A and B comprise C10A/K15A/G22E, and C and D comprise C10A/K15A/S112K (or vice versa);
A and B comprise C10A/K15A/S112D, and C and D comprise C10A/K15A/S112K (or vice versa);
A and B comprise C10A/K15A/S112E, and C and D comprise C10A/K15A/S112K (or vice versa);
A and B comprise C10A/K15A/T119D, and C and D comprise C10A/K15A/S112K (or vice versa);
A and B comprise C10A/K15A/T119E, and C and D comprise C10A/K15A/S I12K (or vice versa);
A and B comprise C10A/K15A/V121D, and C and D comprise C10A/K15A/S112K (or vice versa);
A and B comprise C10A/K15A/V121E, and C and D comprise C10A/K15A/S112K (or vice versa);
A and B comprise C10A/K15A/L17D, and C and D comprise C10A/K15A/T119R (or vice versa);
A and B comprise C10A/K15A/L17E, and C and D comprise C10A/K15A/T119R (or vice versa);
A and B comprise C10A/K15A/V20D, and C and D comprise C10A/K15A/T119R (or vice versa);
A and B comprise C10A/K15A/V20E, and C and D comprise C10A/K15A/T119R (or vice versa);
A and B comprise C10A/K15A/G22D, and C and D comprise C10A/K15A/T119R (or vice versa);
A and B comprise C10A/K15A/G22E, and C and D comprise C10A/K15A/T119R (or vice versa);
A and B comprise C10A/K15A/S112D, and C and D comprise C10A/K15A/T119R (or vice versa); A and B comprise C10A/K15A/S112E, and C and D comprise C10A/K15A/T119R (or vice versa);
A and B comprise C10A/K15A/T119D, and C and D comprise C10A/K15A/T119R (or vice versa);
A and B comprise C10A/K15A/T119E, and C and D comprise C10A/K15A/T119R (or vice versa);
A and B comprise C10A/K15A/V121D, and C and D comprise C10A/K15A/T119R (or vice versa);
A and B comprise C10A/K15A/V121E, and C and D comprise C10A/K15A/T119R (or vice versa);
A and B comprise C10A/K15A/L17D, and C and D comprise C10A/K15A/T119K (or vice versa);
A and B comprise C10A/K15A/L17E, and C and D comprise C10A/K15A/T119K (or vice versa);
A and B comprise C10A/K15A/V20D, and C and D comprise C10A/K15A/T119K (or vice versa);
A and B comprise C10A/K15A/V20E, and C and D comprise C10A/K15A/T119K (or vice versa);
A and B comprise C10A/K15A/G22D, and C and D comprise C10A/K15A/T119K (or vice versa);
A and B comprise C10A/K15A/G22E, and C and D comprise C10A/K15A/T119K (or vice versa);
A and B comprise C10A/K15A/S112D, and C and D comprise C10A/K15A/T119K (or vice versa);
A and B comprise C10A/K15A/S112E, and C and D comprise C10A/K15A/T119K (or vice versa);
A and B comprise C10A/K15A/T119D, and C and D comprise C10A/K15A/T119K (or vice versa);
A and B comprise C10A/K15A/T119E, and C and D comprise C10A/K15A/T119K (or vice versa);
A and B comprise C10A/K15A/V121D, and C and D comprise C10A/K15A/T119K (or vice versa);
A and B comprise C10A/K15A/V121E, and C and D comprise C10A/K15A/T119K (or vice versa);
A and B comprise C10A/K15A/L17D, and C and D comprise C10A/K15A/V121R (or vice versa);
A and B comprise C10A/K15A/L17E, and C and D comprise C10A/K15A/V121R (or vice versa);
A and B comprise C10A/K15A/V20D, and C and D comprise C10A/K15A/V121R (or vice versa);
A and B comprise C10A/K15A/V20E, and C and D comprise C10A/K15A/V121R (or vice versa);
A and B comprise C10A/K15A/G22D, and C and D comprise C10A/K15A/V121R (or vice versa);
A and B comprise C10A/K15A/G22E, and C and D comprise C10A/K15A/V121R (or vice versa);
A and B comprise C10A/K15A/S112D, and C and D comprise C10A/K15A/V121R (or vice versa);
A and B comprise C10A/K15A/S112E, and C and D comprise C10A/K15A/V121R (or vice versa);
A and B comprise C10A/K15A/T119D, and C and D comprise C10A/K15A/V121R (or vice versa);
A and B comprise C10A/K15A/r119E, and C and D comprise C10A/K15A/V121R (or vice versa);
A and B comprise C10A/K15A/V121D, and C and D comprise C10A/K15A/V121R (or vice versa);
A and B comprise C10A/K15A/V121E, and C and D comprise C10A/K15A/V121R (or vice versa);
A and B comprise C10A/K15A/L17D, and C and D comprise C10A/K15A/V121K (or vice versa);
A and B comprise C10A/K15A/L17E, and C and D comprise C10A/K15A/V121K (or vice versa);
A and B comprise C10A/K15A/V20D, and C and D comprise C10A/K15A/V121K (or vice versa);
A and B comprise C10A/K15A/V20E, and C and D comprise C10A/K15A/V121K (or vice versa);
A and B comprise C10A/K15A/G22D, and C and D comprise C10A/K15A/V121K (or vice versa);
A and B comprise C10A/K15A/G22E, and C and D comprise C10A/K15A/V121K (or vice versa); A and B comprise C10A/K15A/S112D, and C and D comprise C10A/K15A/V121K (or vice versa);
A and B comprise C10A/K15A/S112E, and C and D comprise C10A/K15A/V121K (or vice versa);
A and B comprise C10A/K15A/T119D, and C and D comprise C10A/K15A/V121K (or vice versa);
A and B comprise C10A/K15A/T119E, and C and D comprise C10A/K15A/V121K (or vice versa);
A and B comprise C10A/K15A/V121D, and C and D comprise C10A/K15A/V121K (or vice versa); or A and B comprise C10A/K15A/V121E, and C and D comprise C10A/K15A/V121K (or vice versa).

In other specific embodiments, the present invention relates to a TTR protein complex wherein each of A, B, C, and D comprise the amino acid sequence of SEQ ID NO: 1 with the following mutations:

A and B comprise C10A/K15A/L17D, and C and D comprise C10A/K15A/V121R (or vice versa);
A and B comprise C10A/K15A/L17D, and C and D comprise C10A/K15A/V121K (or vice versa);
A and B comprise C10A/K15A/L17E, and C and D comprise C10A/K15A/V121R (or vice versa);
A and B comprise C10A/K15A/V20D, and C and D comprise C10A/K15A/V20R (or vice versa);
A and B comprise C10A/K15A/V20D, and C and D comprise C10A/K15A/V20K (or vice versa);
A and B comprise C10A/K15A/V20E, and C and D comprise C10A/K15A/V20R (or vice versa);
A and B comprise C10A/K15A/V20E, and C and D comprise C10A/K15A/V20K (or vice versa);
A and B comprise C10A/K15A/T119D, and C and D comprise C10A/K15A/L17R (or vice versa);
A and B comprise C10A/K15A/T119D, and C and D comprise C10A/K15A/L17K (or vice versa); or A and B comprise C10A/K15A/V121E, and C and D comprise C10A/K15A/L17K (or vice versa).

In some embodiments, A and B comprise two mutations in SEQ ID NO: 1, wherein said mutations are selected from the list comprising: L17D/V20D, L17D/V20E, L17E/V20D, L17E/V20E, L17D/T119D, L17D/V121E, L17E/T119D, L17E/V121E, V20D/T119D, V20D/V121E, V20E/T119D, and V20E/V121E.

In some embodiments, C and D comprise two mutations in SEQ ID NO: 1, wherein said mutations are selected from the list comprising: L17K/V20K, L17K/V20R, L17R/V20K, L17R/V20R, L17K/V121K, L17K/V121R, L17R/V121K, L17R/V121R, V20K/V121K, V20K/V121R, V20R/V121K, and V20R/V121R.

The present invention also comprises embodiments wherein each of A, B, C, and D in the TTR protein complex comprise the amino acid sequence of SEQ ID NO: 1 with the following mutations:

A and B comprise C10A/K15A/L17D/V20D, and C and D comprise C10A/K15A/L17K/V20K (or vice versa);
A and B comprise C10A/K15A/L17D/V20E, and C and D comprise C10A/K15A/L17K/V20R (or vice versa);
A and B comprise C10A/K15A/L17E/V20D, and C and D comprise C10A/K15A/L17R/V20K (or vice versa); A and B comprise C10A/K15A/L17E/V20E, and C and D comprise C10A/K15A/L17R/V20R (or vice versa);
A and B comprise C10A/K15A/L17D/T119D, and C and D comprise C10A/K15A/L17K/V121K (or vice versa);
A and B comprise C10A/K15A/L17D/V121E, and C and D comprise C10A/K15A/L17K/V121R (or vice versa);
A and B comprise C10A/K15A/L17E/T119D, and C and D comprise C10A/K15A/L17RN 121K (or vice versa);
A and B comprise C10A/K15A/L17E/V121E, and C and D comprise C10A/K15A/L17R/V121R (or vice versa);
A and B comprise C10A/K15A/V20D/T119D, and C and D comprise C10A/K15A/V20K/V121K (or vice versa);
A and B comprise C10A/K15A/V20D/V121E, and C and D comprise C10A/K15A/V20K/V121R (or vice versa);
A and B comprise C10A/K15A/V20E/T119D, and C and D comprise C10A/K15A/V20R/V121K (or vice versa); or A and B comprise C10A/K15A/V20E/V121E, and C and D comprise C10A/K15A/V20R/V121R (or vice versa)

In some embodiments, the TTR protein complex is attached to 1, 2, 3, 4, 5, 6, 7, or 8 bioactive proteins, peptides, or small molecules. In some embodiments, the TTR protein complex is attached to 1, 2, 3, 4, 5, 6, 7, or 8 antigen binding proteins or peptides. In other embodiments, the TTR protein complex is attached to 1, 2, 3, or 4 antigen binding proteins or peptides. The antigen binding proteins or peptides may be attached to the TTR protein complex at the C-terminus of a TTR subunit or the N-terminus of a TTR subunit. In addition, the TTR protein complex may be directly attached to 1, 2, 3, 4, 5, 6, 7, or 8 antigen binding proteins or peptides; or may be attached to 1, 2, 3, 4, 5, 6, 7, or 8 antigen binding proteins or peptides via a linker. In particular embodiments, the TTR protein complex is directly attached to 1, 2, 3, or 4 antigen binding proteins or peptides; or is attached to 1, 2, 3, or 4 antigen binding proteins or peptides via a linker.

The linker may be an amino acid-based linker comprising 1, 2, 3, 4, 5, 6, 7, 8, 9, 10, I1, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In other embodiments, the linker is an amino acid-based linker comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids. In other embodiments, the linker is an amino acid-based linker comprising 1, 2, 3, 4, 5, 6, 7, 8, 9, 10, 11, 12, 13, 14, 15, 16, 17, 18, 19, or 20 amino acids. In yet other embodiments, the linker is an amino acid-based linker comprising 2, 3, 4, 5, 6, 7, 8, 9, or 10 amino acids. In particular embodiments, the linker is G, GG, GGG GGGG, GGGGG, GGGGGG, GGGGGGG, GGGGGGGG, GGGGGGGGG, or GGGGGGGGGG. In other particular embodiments, the linker is selected from the list comprising: GG, GGGG, GGGSGG, GGGSGGGG, and GGAGGGAGGG.

Other suitable linkers include a G(GₓB_{y})ᵣG_{z} linker wherein G=glycine; B=any amino acid; x=1-15; y=1-5; z=1-15; and r=1-20. In another embodiment, the linker is a G(GₓB_{y})ᵣG_{z} linker wherein B=Q, S, A, E, P, T, K, R, D or N; x=4; y=1; z=4; and r=1.

In some embodiments of the present invention, the TTR protein complex is attached to two antigen binding proteins, wherein each antigen binding protein binds a different antigen. In other embodiments of the present invention, the TTR protein complex is attached to four antigen binding proteins, wherein the antigen binding proteins bind to at least two different antigens (e.g., one antigen binding protein binds to a first antigen and three antigen binding proteins bind to a second antigen; or two antigen binding proteins bind to a first antigen and two antigen binding proteins bind to a second antigen).

The antigen binding proteins may be antibodies. In other embodiments, the antigen binding proteins are Fabs or scFvs. In a particular embodiment the antigen binding proteins are Fabs. In other embodiments, the antigen binding proteins are a mixture of antibodies and Fabs.

The present invention also includes pharmaceutical compositions comprising any of the TTR protein complexes discussed herein.

In addition, the present invention includes methods of treating cancer using any of the TTR protein complexes discussed herein. The TTR protein complexes of the present invention may be used in the treatment of cancer. The present invention also includes any of the TTR protein complexes discussed herein for use in the treatment of cancer.

In another embodiment, the present invention includes one or more isolated nucleic acid(s) encoding any of the TTR protein complexes discussed herein. In addition, the present invention includes expression vector(s) comprising a nucleic acid encoding any of the TTR protein complexes discussed herein. The present invention further includes a recombinant host cell comprising such nucleic acid(s) or vector(s). In some embodiments, the host cell is a Chinese hamster ovary (CHO) cell, E5 cell, baby hamster kidney (BHK) cell, monkey kidney (COS) cell, human hepatocellular carcinoma cell, or human embryonic kidney 293 (HEK 293) cell.

In some embodiments, the present invention relates to a method of making a TTR protein complex described herein, wherein the method comprises: a) culturing a recombinant host cell; and b) isolating the TTR protein complex from the culture.

### BRIEF DESCRIPTION OF THE DRAWINGS

FIG. 1. FIG. 1 is a schematic representation of the TTR protein complex (constructs or fusions proteins) of the present invention. FIG. 1a depicts the four TTR subunits that make up the TTR complexes of the present invention. FIG. 1b is an exemplary TTR antibody heterodimer fusion protein, where the C-terminus of both antibody heavy chains is linked to the N-terminus of each TTR subunit. In this example, the two antibodies bind different epitopes either on the same or different protein. FIG. 1c is an exemplary TTR antibody/Fab heterotrimer fusion protein. In this example, the antibody binds a first epitope and the Fabs bind a second epitope either on the same or different protein. FIG. 1d is an exemplary TTR Fab heterotetramer fusion protein, where the C-terminus of each Fab is linked to the N-terminus of each TTR subunit. In this example, two Fabs bind a first epitope and two Fabs bind a second epitope either on the same or different protein. Each of FIGS. 1b-1d Shows an Optional Linker Between the heavy chain and TTR.

FIG. 2. FIGS. 2a-e represent exemplary heteromeric TTR Ab, TTR Fab, and TTR Ab/Fab protein complexes. Any antigen binding protein (e.g., Fab, antibody, scFv, scFab) or proteins such as enzymes can be used in the TTR protein complexes of the present invention. The “+” and “−” signs in FIG. 2c indicate Fc charge pairs which allow for consistent attachment of one TTR subunit per whole antibody. FIGS. 2a-e also recite a shorthand description for each of the protein complexes (e.g., [[Fab “A”]-[negative TTR]]₂: [[positive TTR]-[Fab “B”]]₂) wherein “A” refers to a first target and “B” refers to a second target. A “−” denotes a single point of attachment between two moieties (e.g., [positive TTR] and [Fab “B”]), while a “=” denotes two points of attachment between two moieties (e.g., [655-341 Ab] and [negative TTR]). Notably, the shorthand does not imply N→C or C→N orientation, but rather is shorthand description of the molecule from “left to right.” A second form of shorthand is also depicted. For example, the FIG. 2a construct can be noted as a “4×-Fab-TTR” indicated that 4 Fabs are attached to the TTR complex. Similarly, the FIG. 2b and FIG. 2e constructed can be noted as “2×-Ab-TTR” and “4×-Ab-TTR,” respectively.

FIG. 3. FIG. 3 depicts the interface between the TTR monomers which form two sets of TTR dimers (left side) and the interface between the TTR dimers which form a TTR tetramer (right side). As can be seen, the interface between the TTR monomers differs from the interface between the TTR dimers.

FIG. 4. FIG. 4 depicts the 18 TTR charge variants (C10A/K15A/XX) of TTR (SEQ ID NO: 1) that were made to evaluate whether charge mutations would result in substantial repulsion of the TTR dimer/dimer interface.

FIG. 5. FIG. 5 depicts how various TTR dimer/dimer interfaces (with mutations) responded to the presence of SDS (chaotrope) with and without heating.

FIG. 6. FIG. 6 depicts the effect of non-denaturing conditions on TTR variants. The TTR variant, yield, whether the TTR was present as a tetramer or dimer (according to SEC and SDS-PAGE analysis), and the TTR melting temperature are noted.

FIG. 7. FIG. 7 depicts an assessment of TTR heterotetramer formation by both SEC (top) and SDS-PAGE (bottom). The SEC (top) analysis indicates that many of the variant pairings had a propensity to form heteromultimers as indicated by a non-zero value (% of molecules w/retention time consistent w/TTR tetramer). In addition, many of the TTR heterotetramers were resistant to breakdown by chaotropic SDS as indicated by the SDS-PAGE results.

FIG. 8. FIG. 8 depicts a further SDS-PAGE assessment of positive/negative pairings that demonstrated a high propensity to form stable TTR tetramers. The TTR heterotetramers are resistant to SDS induced denaturation, which is an indicator of good stability. For each pairing, [1] the negative (i.e., basic) variant; [2] the positive (i.e., acidic) variant; [3] the combination of negative and positive variants (which should form a tetramer); and [4] the combination of negative and positive variants exposed to caspase, were evaluated.

FIG. 9. FIG. 9 depicts the evaluation of TTR heterotetramers comprising the L17R/T119D, L17K/T119D, L17K/V121E, V20R/V20D, V20R/V20E, V20K/V20D, V20K/V20E, V121R/L17D, V121R/L17E, and V121K/L17D pairings when exposed to pH 5.0 conditions to determine whether they could maintain their tetrameric state (via SEC) in conditions similar to those found in pharmaceutical formulations.

FIG. 10. FIG. 10 depicts the evaluation of TTR heterotetramers melting temperatures. In each case (mutants noted in figure), the TTR heterotetramer was stable to at least 92° C. indicating the heterotetramer is thermally stable.

FIG. 11. FIG. 11 depicts the construction of bispecific TTR heterotetramer Ab constructs. An exemplary bispecific TTR heterotetramer Ab construct is shown wherein each heavy chain of the 655-341 Ab (lined fill) attached to the N-terminus of the negative TTR monomers (together forming a negative TTR dimer) and each heavy chain of the DNP-3B1 Ab (solid fill) attached to the N-terminus of the positive TTR monomers (together forming a positive TTR dimer). Four negative TTR variants were fused to the 655-341 Ab and four positive TTR variants were fused to the DNP-3B1 Ab. All Ab-TTR fusions were made without a linker between the Ab and the TTR monomer.

FIG. 12. FIG. 12 depicts that TTR heterotetramer Ab constructs are not efficiently produced when the [655-341 Ab]=[negative TTR]₂ and [positive TTR]₂=[DNP-3B1 Ab] tetramer portions are expressed in separate mammalian cells (293-6E HEK cells).

FIG. 13. FIG. 13 depicts the construction of bispecific TTR heterodimer Ab constructs for an evaluation of the effect of adding a linker between the Ab heavy chain and the TTR monomer on expression of the two tetramer portions in two different mammalian cells.

FIG. 14. FIG. 14 depicts the results (ordered by linker) of the expression of the two Ab-TTR heterodimer portions in two different mammalian cells of bispecific TTR heterotetramer Ab constructs with linkers between the Ab heavy chain and the TTR monomer.

FIG. 15. FIG. 15 depicts additional results (ordered by linker) of the expression of the two Ab-TTR heterodimer portions in two different mammalian cells of bispecific TTR heterotetramer Ab constructs with linkers between the Ab heavy chain and the TTR monomer.

FIG. 16. FIG. 16 depicts the averaged results from FIGS. 14 and 15.

FIG. 17. FIG. 17 depicts the construction of bispecific TTR heterotetramer Fab constructs for an evaluation of Fab TTR fusions in a mammalian cell line (CHO K1).

FIG. 18. FIG. 18 depicts the results of the evaluation of the bispecific TTR heterotetramer Fab constructs in FIG. 17.

FIG. 19. FIG. 19 depicts the retention (SEC) of heteromultimeric molecule 15524 ([655-341 Fab]-[GG]-[TTR(C10A/K15A/L17D)] and [TTR(C10A/K15A/V121R)]-[GG]-[DNP-3B1 Fab]) compared to homomultimeric Ab- and Fab-TTR fusions as well as unfused Abs (each as standards).

FIG. 20. FIG. 20 depicts a confirmation of the molecular mass of the eluting species (from FIG. 19) via SEC coupled MS as consistent with that expected for molecule 15524 (as a heteromultimer).

FIG. 21. FIG. 21 depicts the construction of Ab-containing TTR fusions to determine if co-expression of Ab-containing TTR fusions would lead to the generation of desired TTR heterotetramer [655-341 Ab]=[[LX]-[negative TTR]]₂: [[positive TTR]-[LX]]₂=[DNP-3B1 Ab].

FIG. 22. FIG. 22 depicts that significant amount of the desired TTR heterotetramer was formed for many of the combinations from FIG. 21.

FIG. 23. FIG. 23 depicts the retention (SEC) of heteromultimeric molecule 15539 ([655-341 Ab]=[[GGAGGGAGGG]-[TTR(C10A/K15A/L17D]]₂: [[TTR(C10A/K15A/V121K)]-[GGAGGGAGGG]]₂=[DNP-3B1 Ab]) compared to homomultimeric Ab- and Fab-TTR fusions as well as unfused Abs (each as standards).

FIG. 24. FIG. 24 depicts a confirmation of the molecular mass of the eluting species (from FIG. 23) via SEC coupled MS as consistent with that expected for molecule 15539 (as a heteromultimer).

FIG. 25. FIG. 25 depicts the averaged results from FIG. 22.

FIG. 26. FIG. 26 depicts the evaluation of whether co-expression of the negative and positive TTR variants (4 of each mutation) fused to Abs and Fabs in the same cell line would lead to the production of Ab-Fab-TTR constructs (i.e., [Ab “A”]=[negative TTR]₂: [[positive TTR]=[Fab “B”]]₂ constructs).

FIG. 27. FIG. 27 depicts the results of the expression, purification, and analytics of the Ab-Fab-TTR constructs of FIG. 26.

FIG. 28. FIG. 28 depicts the retention (with SEC) of heteromultimeric molecule 15545 ([655-341 Ab]=[[GGGG]-[TTR(C10A/K15A/V20D)]]₂: [[TTR(C10A/K15A/V20R)]-[GG]-[DNP-3B1-Fab]]₂) compared to homomultimeric Ab- and Fab-TTR fusions as well as unfused Abs (each as standards).

FIG. 29. FIG. 29 depicts a confirmation of the molecular mass of the eluting species (from FIG. 28) via SEC coupled MS as consistent with that expected for molecule 15545 (as a heteromultimer).

FIG. 30. FIG. 30 depicts the averaged results from FIG. 27.

FIG. 31. FIG. 31 depicts the TTR double charge variants (C10A/K15A/XX/YY) of TTR (SEQ ID NO: 1) that were made to evaluate whether double charge mutations would enable increased selectivity of heteromultimers over homomultimers.

FIG. 32. FIG. 32 depicts the expression yield, purification yield, and SEC properties of separately expressed single and double interface mutants.

FIG. 33. FIG. 33 depicts the SEC profile of exemplary single and double interface mutants.

FIG. 34. FIG. 34 depicts the TTR double charge variants (C10A/K15A/XX/YY) of TTR (SEQ ID NO: 1) that were made to evaluate whether double charge mutations would enable increased selectivity of heteromultimers over homomultimers.

FIG. 35. FIG. 35 depicts the SEC properties of the post-purification mixing of the single and double interface mutants sorted by negative mutation.

FIG. 36. FIG. 36 depicts the SEC properties of the separate post-purification mixing of the single and double interface mutants sorted by positive mutation.

FIG. 37. FIG. 37 depicts the SEC profiles of exemplary single and double interface mutants separately and after post-purification mixing.

FIG. 38. FIG. 38 depicts the purification yield and SEC profiles of the single and double interface mutants produced by co-culture of the cell lines.

FIG. 39. FIG. 39 depicts a continuation of the data presented in FIG. 38.

FIG. 40. FIG. 40 depicts the SEC profile of exemplary molecules produced by co-culturing double interface mutants.

### DETAILED DESCRIPTION

The section headings used herein are for organizational purposes only and are not to be construed as limiting the subject matter described.

Unless otherwise defined herein, scientific and technical terms used in connection with the present application have the meanings that are commonly understood by those of ordinary skill in the art. Further, unless otherwise required by context, singular terms shall include pluralities and plural terms shall include the singular.

Generally, nomenclatures used in connection with, and techniques of, cell and tissue culture, molecular biology, immunology, microbiology, genetics and protein and nucleic acid chemistry and hybridization described herein are those well-known and commonly used in the art. The methods and techniques of the present application are generally performed according to conventional methods well known in the art and as described in various general and more specific references that are cited and discussed throughout the present specification unless otherwise indicated. See, e.g., Sambrook et al., Molecular Cloning: A Laboratory Manual, 3rd ed., Cold Spring Harbor Laboratory Press, Cold Spring Harbor, N.Y. (2001), Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are those well-known and commonly used in the art. Standard techniques can be used for chemical syntheses, chemical analyses, pharmaceutical preparation, formulation, and delivery, and treatment of patients.

It should be understood that this invention is not limited to the particular methodology, protocols, and reagents, etc., described herein and as such may vary. The terminology used herein is for the purpose of describing particular embodiments only, and is not intended to limit the scope of the disclosed, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean±1%.

All embodiments narrower in scope in any way than the variations defined by specific paragraphs herein are to be considered included in this disclosure. For example, certain aspects are described as a genus, and it should be understood that every member of a genus can be, individually, an embodiment. Also, aspects described as a genus or selecting a member of a genus should be understood to embrace combinations of two or more members of the genus. It should also be understood that while various embodiments in the specification are presented using “comprising” language, under various circumstances, a related embodiment may also be described using “consisting of” or “consisting essentially of” language.

In this application, the use of “or” means “and/or” unless stated otherwise. Furthermore, the use of the term “including,” as well as other forms, such as “includes” and “included”, is not limited. Also, terms such as “element” or “component” encompass both elements and components comprising one unit and elements and components that comprise more than one subunit unless specifically stated otherwise.

### Definitions

“Amino acid” includes its standard meaning in the art. The twenty naturally-occurring amino acids and their abbreviations follow conventional usage. See, Immunology—A Synthesis, 2nd Edition, (E. S. Golub and D. R. Green, eds.), Sinauer Associates: Sunderland, Mass. (1991), incorporated herein by reference for any purpose. Stereoisomers (e.g., D-amino acids) of the twenty conventional amino acids, unnatural amino acids such as [alpha]-, [alpha]-disubstituted amino acids, N-alkyl amino acids, and other unconventional amino acids may also be suitable components for polypeptides and are included in the phrase “amino acid.” Examples of unconventional amino acids include: 4-hydroxyproline, [gamma]-carboxyglutamate, [epsilon]-N,N,N-trimethyllysine, [epsilon]-N-acetyllysine, O-phosphoserine. N-acetylserine, N-formylmethionine, 3-methylhistidine, 5-hydroxylysine, [sigma]-N-methylarginine, and other similar amino acids and imino acids (e.g., 4-hydroxyproline). In the polypeptide notation used herein, the left-hand direction is the amino terminal direction and the right-hand direction is the carboxyl-terminal direction, in accordance with standard usage and convention.

An “antagonist” as used herein generally refers to a molecule, for example, an antigen binding protein such as provided herein, that can bind an antigen and inhibit, reduce, or eliminate biological signaling associated with the antigen.

As used herein, the term “antibody” refers to a protein having a conventional immunoglobulin format, comprising heavy and light chains, and comprising variable and constant regions. For example, an antibody may be an IgG which is a “Y-shaped” structure of two identical pairs of polypeptide chains, each pair having one “light” (typically having a molecular weight of about 25 kDa) and one “heavy” chain (typically having a molecular weight of about 50-70 kDa). An antibody has a variable region and a constant region. In IgG formats, the variable region is generally about 100-110 or more amino acids, comprises three complementarity determining regions (CDRs), is primarily responsible for antigen recognition, and substantially varies among other antibodies that bind to different antigens. The constant region allows the antibody to recruit cells and molecules of the immune system. The variable region is made of the N-terminal regions of each light chain and heavy chain, while the constant region is made of the C-terminal portions of each of the heavy and light chains. (Janeway et al., “Structure of the Antibody Molecule and the Immunoglobulin Genes”, Immunobiology: The Immune System in Health and Disease, 4^{th} ed. Elsevier Science Ltd./Garland Publishing, (1999)).

Antibodies can comprise any constant region known in the art. Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the present disclosure include all such classes or isotypes of antibodies. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. Accordingly, in exemplary embodiments, the antibody is an antibody of isotype IgA, IgD, IgE, IgG, or IgM, including any one of IgG1, IgG2, IgG3 or IgG4.

The term “antigen” refers to a molecule or a portion of a molecule capable of being bound by a binding agent, such as an antigen binding protein (including, e.g., an antibody), and additionally capable of being used in an animal to produce antibodies capable of binding to that antigen. An antigen may possess one or more epitopes that are capable of interacting with different antigen binding proteins, e.g., antibodies.

An “antigen binding protein” as used herein means any protein that specifically binds a specified target antigen. The term includes polypeptides that include at least one antigen binding region. The term also encompasses antibodies that comprise at least two full-length heavy chains and two full-length light chains, as well as derivatives, variants, fragments, and mutations thereof. An antigen binding protein also includes Fab, Fab′, F(ab′)₂, Fv fragments, domain antibodies such as Nanobodies® and scFvs, as described in more detail below.

An “antigen binding region” or “antigen binding domain” means the portion of a protein, such as an antibody or a fragment, derivative, or variant thereof, that specifically binds to, interacts with, or recognizes a given epitope or site on a molecule (e.g., an antigen). For example, the portion of an antigen binding protein that contains the amino acid residues that interact with an antigen and confer on the antigen binding protein its specificity and affinity for the antigen is referred to as “antigen binding region.” An antigen binding region can include one or more “complementarity determining regions” (“CDRs”). Certain antigen binding regions also include one or more “framework” regions. “Framework” regions can contribute directly to the specific binding of the antigen binding protein, but typically aid in maintaining the proper conformation of the CDRs to promote binding between the antigen binding region and an antigen.

The terms “cancer”, “tumor”, “cancerous”, and “malignant” refer to or describe the physiological condition in mammals that is typically characterized by unregulated cell growth. Examples of cancer include but are not limited to, carcinoma including adenocarcinoma, lymphoma, blastoma, melanoma, sarcoma, and leukemia. More particular examples of such cancers include melanoma, lung cancer, head and neck cancer, renal cell cancer, colon cancer, colorectal cancer, squamous cell cancer, small-cell lung cancer, non-small cell lung cancer, gastrointestinal cancer, Hodgkin's and non-Hodgkin's lymphoma, pancreatic cancer, glioblastoma, glioma, cervical cancer, ovarian cancer, liver cancer such as hepatic carcinoma and hepatoma, bladder cancer, breast cancer, endometrial carcinoma, myeloma (such as multiple myeloma), salivary gland carcinoma, kidney cancer such as renal cell carcinoma and Wilms' tumors, basal cell carcinoma, prostate cancer, vulval cancer, thyroid cancer, testicular cancer, and esophageal cancer.

The terms “CDR.” and its plural “CDRs” (also referred to as “hypervariable regions”), refer to the complementarity determining region of a protein, such as an antibody or a fragment, derivative, or variant thereof. The light chain variable region and the heavy chain variable region each contain three CDRs. For example, the light chain variable region contains the following CDRs: CDR-L1, CDR-L2 and CDR-L3; and the heavy chain variable region contains the following CDRs: CDR-H1, CDR-H2 and CDR-H3. CDRs contain most of the residues responsible for specific interactions of the antibody with the antigen and hence contribute to the functional activity of an antibody molecule. CDRs are the main determinants of antigen specificity.

The exact definitional CDR boundaries and lengths are subject to different classification and numbering systems. CDRs may therefore be referred to by Kabat, Chothia, contact or any other boundary definitions, including the numbering system described herein. The Kabat numbering scheme (system) is a widely adopted standard for numbering the amino acid residues of an antibody variable domain in a consistent manner and is the preferred scheme applied in the present invention as also mentioned elsewhere herein. Additional structural considerations can also be used to determine the canonical structure of an antibody. For example, those differences not fully reflected by Kabat numbering can be described by the numbering system of Chothia et al. and/or revealed by other techniques, for example, crystallography and two- or three-dimensional computational modeling. Despite differing boundaries, each of these systems has some degree of overlap in what constitutes a CDR within the variable sequences. CDR definitions according to these systems may therefore differ in length and boundary areas with respect to the adjacent framework region. See, e.g., Kabat (an approach based on cross-species sequence variability), Chothia (an approach based on crystallographic studies of antigen-antibody complexes), and/or MacCallum (Kabat et al., loc. cit.; Chothia et al., J. Mol. Biol, 1987, 196: 901-917; and MacCallum et al., J. Mol. Biol, 1996, 262: 732). Still another standard for characterizing the antigen binding site is the AbM definition used by Oxford Molecular's AbM antibody modeling software. See, e.g., Protein Sequence and Structure Analysis of Antibody Variable Domains. In: Antibody Engineering Lab Manual (Ed.: Duebel, S. and Kontermann, R., Springer-Verlag, Heidelberg). For a review of the antibody structure, see Antibodies: A Laboratory Manual, Cold Spring Harbor Laboratory, eds. Harlow et al., 1988.

Typically, CDRs form a loop structure that can be classified as a canonical structure. The term “canonical structure” refers to the main chain conformation that is adopted by the antigen binding (CDR) loops. From comparative structural studies, it has been found that five of the six antigen binding loops have only a limited repertoire of available conformations. Each canonical structure can be characterized by the torsion angles of the polypeptide backbone. Correspondent loops between antibodies may, therefore, have very similar three dimensional structures, despite high amino acid sequence variability in most parts of the loops (Chothia and Lesk, J. Mol. Biol., 1987, 196: 901; Chothia et al., Nature, 1989, 342: 877; Martin and Thornton, J. Mol. Biol, 1996, 263: 800). Furthermore, there is a relationship between the adopted loop structure and the amino acid sequences surrounding it. The conformation of a particular canonical class is determined by the length of the loop and the amino acid residues residing at key positions within the loop, as well as within the conserved framework (i.e., outside of the loop). Assignment to a particular canonical class can therefore be made based on the presence of these key amino acid residues.

The term “compete” when used in the context of antigen binding proteins (e.g., antibodies or fragments thereof) that compete for the same epitope means competition between antigen binding proteins and is determined by an assay in which the antigen binding protein (e.g., antibody or fragment thereof) under test prevents or inhibits specific binding of a reference antigen binding protein to a common antigen. Numerous types of competitive binding assays can be used, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using I-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of purified antigen bound to a solid surface or cells expressing the antigen, an unlabelled test antigen binding protein and a labeled reference antigen binding protein. Competitive inhibition is measured by determining the amount of label bound to the solid surface or cells in the presence of the test antigen binding protein. Usually the test antigen binding protein is present in excess. Antigen binding proteins identified by competition assay include antigen binding proteins binding to the same epitope as the reference antigen binding proteins and antigen binding proteins binding to an adjacent epitope sufficiently proximal to the epitope bound by the reference antigen binding protein for steric hindrance to occur. Additional details regarding methods for determining competitive binding are provided herein. For instance, in one embodiment, competition is determined according to a BiaCore assay. Usually, when a competing antigen binding protein is present in excess, it will inhibit specific binding of a reference antigen binding protein to a common antigen by at least 20%, 25%, 30%, 35%, 40%, 45%, 50%, 55%, 60%, 65%, 70% or 75%. In some instances, binding is inhibited by at least 80%, 85%, 90%, 95%, or 97% or more.

The term “control sequence” refers to a polynucleotide sequence that can affect the expression and processing of coding sequences to which it is ligated. The nature of such control sequences may depend upon the host organism. In particular embodiments, control sequences for prokaryotes may include a promoter, a ribosomal binding site, and a transcription termination sequence. For example, control sequences for eukaryotes may include promoters comprising one or a plurality of recognition sites for transcription factors, transcription enhancer sequences, and transcription termination sequences. “Control sequences” can include leader sequences and/or fusion partner sequences.

A “derivative” of a polypeptide is a polypeptide that has been modified (e.g., chemically) in some manner distinct from insertion, deletion, or substitution variants, e.g., via conjugation to another chemical moiety.

A “domain antibody” is an immunologically functional immunoglobulin fragment containing only the variable region of a heavy chain or the variable region of a light chain. Examples of domain antibodies include Nanobodies®. In some instances, two or more V_{H} regions are covalently joined with a peptide linker to create a bivalent domain antibody. The two V_{H} regions of a bivalent domain antibody may target the same or different antigens.

An “effective amount” is generally an amount sufficient to reduce the severity and/or frequency of symptoms, eliminate the symptoms and/or underlying cause, prevent the occurrence of symptoms and/or their underlying cause, and/or improve or remediate the damage that results from or is associated with cancer. In some embodiments, the effective amount is a therapeutically effective amount or a prophylactically effective amount. A “therapeutically effective amount” is an amount sufficient to remedy a disease state (e.g. cancer) or symptoms, particularly a state or symptoms associated with the disease state, or otherwise prevent, hinder, retard or reverse the progression of the disease state or any other undesirable symptom associated with the disease in any way whatsoever. A “prophylactically effective amount” is an amount of a pharmaceutical composition that, when administered to a subject, will have the intended prophylactic effect, e.g., preventing or delaying the onset (or reoccurrence) of cancer, or reducing the likelihood of the onset (or reoccurrence) of cancer or cancer symptoms. The full therapeutic or prophylactic effect does not necessarily occur by administration of one dose, and may occur only after administration of a series of doses. Thus, a therapeutically or prophylactically effective amount may be administered in one or more administrations.

The term “epitope” refers to the portion of an antigen capable of being recognized and specifically bound by an antigen binding protein (e.g., an antibody). In the context of polypeptides, epitopes can be formed from contiguous amino acids or non-contiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more typically, at least 5 or 8-10 amino acids in a unique spatial conformation. A “linear epitope” or a “sequential epitope” is an epitope that is recognized by an antigen binding protein (e.g., an antibody) by its linear sequence of amino acids, or primary structure. A “conformational epitope” or a “nonsequential epitope” is an epitope that is recognized by an antigen binding protein (e.g., an antibody) via its tertiary structure. The residues that constitute these epitopes may not be contiguous in the primary amino acid sequence but are brought close together in the tertiary structure of the molecule. Linear and conformational epitopes generally behave differently when a protein is denatured, fragmented, or reduced.

The term “expression vector” or “expression construct” refers to a vector that is suitable for transformation of a host cell and contains nucleic acid sequences that direct and/or control (in conjunction with the host cell) expression of one or more heterologous coding regions operatively linked thereto. An expression construct may include, but is not limited to, sequences that affect or control transcription, translation, and, if introns are present, affect RNA splicing of a coding region operably linked thereto.

A “Fab fragment” or “Fab” is comprised of one light chain and the C_{H}1 and variable regions of one heavy chain. The heavy chain of a Fab molecule cannot form a disulfide bond with another heavy chain molecule.

A “Fab′ fragment” or “Fab′” contains one light chain and a portion of one heavy chain that contains the V_{H} domain and the C_{H}1 domain and also the region between the C_{H}1 and C_{H}2 domains, such that an interchain disulfide bond can be formed between the two heavy chains of two Fab′ fragments to form an F(ab′)₂ molecule.

A “F(ab′)₂ fragment” or “F(ab′)₂” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

An “Fc region” contains two heavy chain fragments comprising the C_{H}2 and C_{H}3 domains of an antibody. The two heavy chain fragments are held together by two or more disulfide bonds and by hydrophobic interactions of the C_{H}3 domains.

The “Fv region” comprises the variable regions from both the heavy and light chains, but lacks the constant regions.

The term “heavy chain” as used with respect to an antigen binding protein, antibody, or fragment thereof, includes a full-length heavy chain. A full-length heavy chain includes a variable region domain (V_{H}) and three constant region domains (C_{H}1, C_{H}2, and C_{H}3). The V_{H} domain is at the amino-terminus of the polypeptide, and the C_{H} domains are at the carboxyl-terminus, with the C_{H}3 being closest to the carboxy-terminus of the polypeptide. Heavy chains may be of any isotype such as IgG (including IgG1, IgG2, IgG3 and IgG4 subtypes), IgA (including IgA1 and IgA2 subtypes), IgM and IgE. Fragments of heavy chains have sufficient variable region sequence to confer binding specificity.

“Hematological cancers” are cancer that begins in blood-forming tissue, such as the bone marrow, or in the cells of the immune system. Examples of hematologic cancer are leukemia, lymphoma, and multiple myeloma.

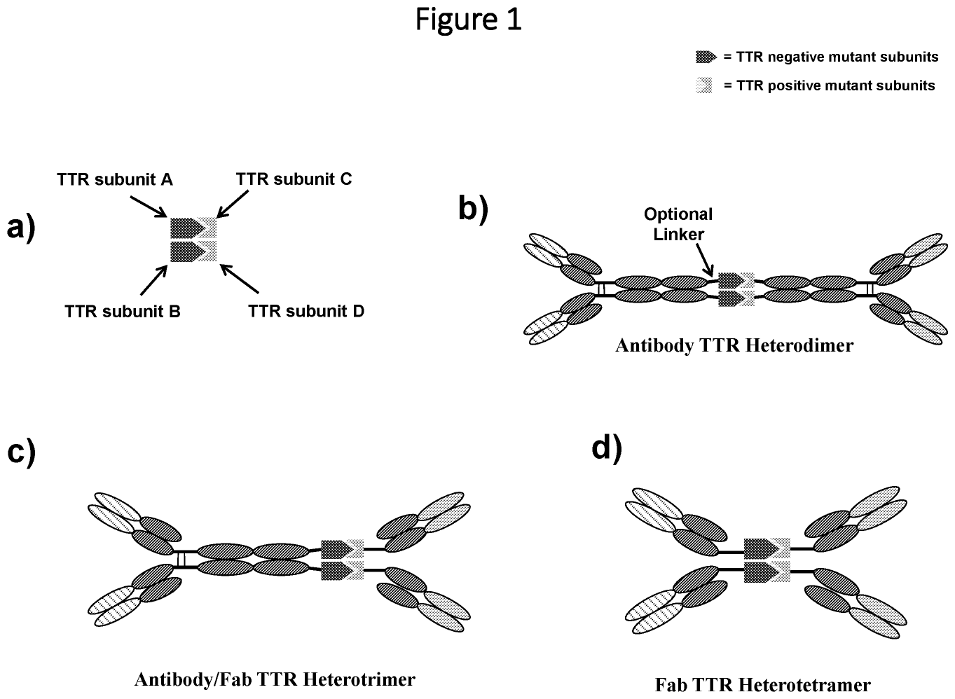

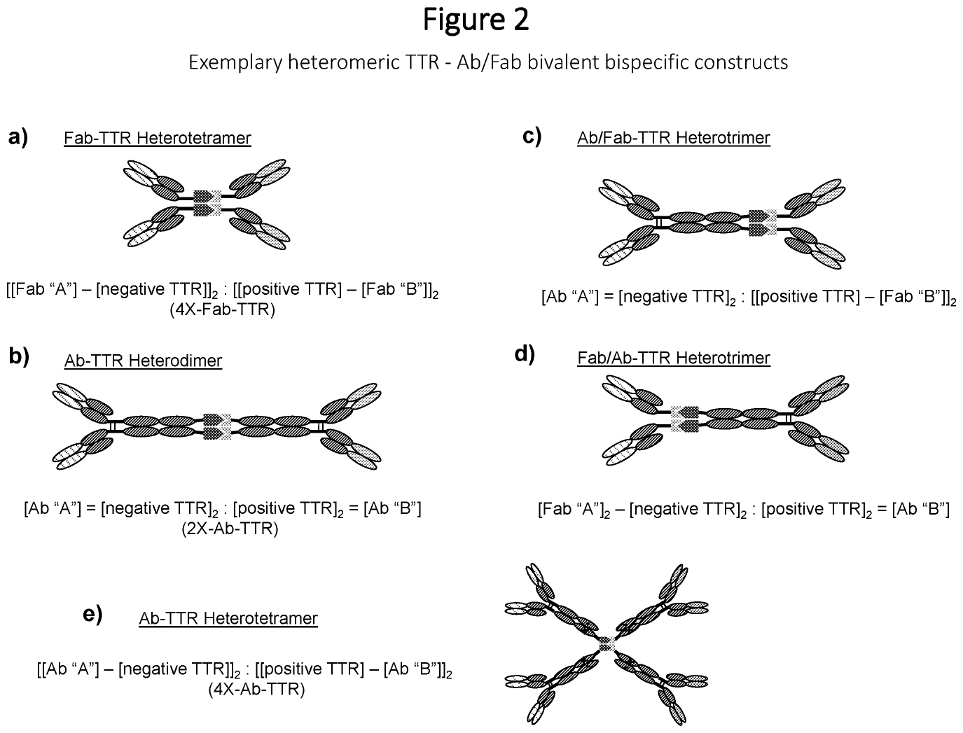

The term “heterodimer fusion protein” or “heterodimer protein complex” refers to a fusion protein comprising two different proteins (e.g., antigen binding proteins; peptides such as agonist peptides; and agonist protein domains). In a particular example, the heterodimer can be a TTR heterodimer fusion protein which comprises two different antigen binding proteins (e.g., two different antibodies) linked via a TTR protein, as described herein. In another example, the heterodimer can be a TTR heterodimer fusion protein which comprises one antibody and one Fab linked via a TTR protein, as described herein. Exemplary heterodimer fusion proteins are depicted in FIGS. 1b and 2b.

The term “heterotrimer fusion protein” or “heterotrimer protein complex” refers to a fusion protein comprising three different proteins (e.g., antigen binding proteins; peptides such as agonist peptides; and agonist protein domains). In a particular example, the heterotrimer can be a TTR heterotrimer fusion protein which comprises an antibody and two Fabs linked via a TTR protein, as described herein (see, e.g., FIG. 2c).

The term “heterotetramer fusion protein” or “heterotetramer protein complex” refers to a fusion protein comprising four different proteins (e.g., antigen binding proteins; peptides such as agonist peptides; and agonist protein domains). In a particular example, the heterotetramer fusion protein is a TTR heterotetramer fusion protein wherein the, e.g., antibodies, Fabs, or mixtures thereof are linked via a TTR protein, as described herein. Examples include where the antigen binding protein is an antibody (see, e.g., FIG. 2e) or a Fab (see, e.g., FIGS. 1d and 2a). In a particular example, the heterotetramer fusion protein is a TTR heterotetramer fusion protein wherein the, e.g., antibodies, Fabs, or mixtures thereof are linked via a TTR protein, as described herein.

The term “host cell” means a cell that has been transformed with a nucleic acid sequence and thereby expresses a gene of interest. The term includes the progeny of the parent cell, whether or not the progeny is identical in morphology or in genetic make-up to the original parent cell, so long as the gene of interest is present.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) must be addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073.

In calculating percent identity, the sequences being compared are aligned in a way that gives the largest match between the sequences. The computer program used to determine percent identity is the GCG program package, which includes GAP (Devereux et al., 1984, Nucl. Acid Res. 12:387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm.

Recommended parameters for determining percent identity for polypeptides or nucleotide sequences using the GAP program are the following:

Algorithm: Needleman et al., 1970, J. Mol. Biol. 48:443-453;

Comparison matrix: BLOSUM 62 from Henikoff et al., 1992, supra;

Gap Penalty: 12 (but with no penalty for end gaps)

Gap Length Penalty: 4

Threshold of Similarity: 0

Certain alignment schemes for aligning two amino acid sequences may result in matching of only a short region of the two sequences, and this small aligned region may have very high sequence identity even though there is no significant relationship between the two full-length sequences. Accordingly, the selected alignment method (GAP program) can be adjusted if so desired to result in an alignment that spans at least 50 contiguous amino acids of the target polypeptide.

The phrase “immune modulator” refers to a molecule that induces, enhances or suppresses an immune response. An immune activator is a molecule that induces or amplifies an immune response. An immune suppressor is a molecule that reduces or suppresses an immune response. Thus, an activation immunotherapy is a therapy that involves administering a molecule(s) to induce or enhance a subject's immune system. A suppression immunotherapy is a therapy in which a subject is treated with a molecule(s) to reduce or suppress the subject's immune system.

The term “fragment” of an antibody or immunoglobulin chain (heavy or light chain), as used herein, is an antigen binding protein comprising a portion (regardless of how that portion is obtained or synthesized) of an antibody that lacks at least some of the amino acids present in a full-length chain but which is capable of specifically binding to an antigen. Such fragments are biologically active in that they bind specifically to the target antigen and can compete with other antigen binding proteins, including intact antibodies, for binding to a given epitope. In one aspect, such a fragment will retain at least one CDR present in the full-length light or heavy chain, and in some embodiments will comprise a single heavy chain and/or light chain or portion thereof. These biologically active fragments may be produced by recombinant DNA techniques, or may be produced by enzymatic or chemical cleavage of antigen binding proteins, including intact antibodies. Immunologically functional immunoglobulin fragments include, but are not limited to, Fab, Fab′, F(ab′)₂, Fv, domain antibodies and scFvs, and may be derived from any mammalian source, including but not limited to human, mouse, rat, camelids or rabbit. It is contemplated further that a functional portion of the antigen binding proteins disclosed herein, for example, one or more CDRs, could be covalently bound to a second protein or to a small molecule to create a therapeutic agent directed to a particular target in the body or having a prolonged serum half-life.

An “isolated nucleic acid molecule” means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty other proteins or portions thereof, or may include operably linked regulatory sequences that control expression of the coding region of the recited nucleic acid sequences, and/or may include vector sequences.

The term “isolated polypeptide,” “purified polypeptide,” “isolated protein” or “purified protein” as used herein, is intended to refer to a composition, isolatable from other components, wherein the polypeptide is purified to any degree relative to its naturally-obtainable state. A purified polypeptide therefore also refers to a polypeptide that is free from the environment in which it may naturally occur. Generally. “purified” will refer to a polypeptide composition that has been subjected to fractionation to remove various other components, and which composition substantially retains its expressed biological activity. Where the term “substantially purified” is used, this designation will refer to a peptide or polypeptide composition in which the polypeptide or peptide forms the major component of the composition, such as constituting about 50%, about 60%, about 70%, about 80%, about 90%, about 95% or more of the proteins in the composition.

The term “light chain” as used with respect to an antigen binding protein, antibody, or fragments thereof, includes a full-length light chain. A full-length light chain includes a variable region domain (V_{L}) and a constant region domain (C_{L}). The variable region domain of the light chain is at the amino-terminus of the polypeptide. Light chains include kappa chains and lambda chains. Fragments of light chains have sufficient variable region sequence to confer binding specificity.

The term “naturally occurring” as used throughout the specification in connection with biological materials such as polypeptides, nucleic acids, host cells, and the like, refers to materials which are found in nature.

The term “oligonucleotide” means a polynucleotide comprising 200 or fewer nucleotides. In some embodiments, oligonucleotides are 10 to 60 bases in length. In other embodiments, oligonucleotides are 10, 11, 12, 13, 14, 15, 16, 17, 18, 19, or 20 to 40 nucleotides in length. Oligonucleotides may be single stranded or double stranded, e.g., for use in the construction of a mutant gene. Oligonucleotides may be sense or antisense oligonucleotides. An oligonucleotide can include a label, including a radiolabel, a fluorescent label, a hapten or an antigenic label, for detection assays. Oligonucleotides may be used, for example, as PCR primers, cloning primers or hybridization probes.

As used herein. “operably linked” means that the components to which the term is applied are in a relationship that allows them to carry out their inherent functions under suitable conditions. For example, a control sequence in a vector that is “operably linked” to a protein coding sequence is ligated thereto so that expression of the protein coding sequence is achieved under conditions compatible with the transcriptional activity of the control sequences.

The term “polynucleotide” or “nucleic acid” includes both single-stranded and double-stranded nucleotide polymers. The nucleotides comprising the polynucleotide can be ribonucleotides or deoxyribonucleotides or a modified form of either type of nucleotide. The modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phoshoraniladate and phosphoroamidate.

Unless specified otherwise, the left-hand end of any single-stranded polynucleotide sequence discussed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences;” sequence regions on the DNA strand having the same sequence as the RNA transcript that are 3′ to the 3′ end of the RNA transcript are referred to as “downstream sequences.”

The terms “polypeptide” or “protein” are used interchangeably herein to refer to a polymer of amino acid residues. The terms also apply to amino acid polymers in which one or more amino acid residues is an analog or mimetic of a corresponding naturally occurring amino acid, as well as to naturally occurring amino acid polymers. The terms can also encompass amino acid polymers that have been modified, e.g., by the addition of carbohydrate residues (to form glycoproteins), or by phosphorylation. Polypeptides and proteins can be produced by a naturally-occurring and non-recombinant cell or by a genetically-engineered or recombinant cell, and can comprise molecules having the amino acid sequence of the native protein, or molecules having deletions from, additions to, and/or substitutions of one or more amino acids of the native sequence. The term “polypeptide fragment” refers to a polypeptide that has an amino-terminal deletion, a carboxyl-terminal deletion, and/or an internal deletion as compared with the full-length protein. Such fragments may also contain modified amino acids as compared with the full-length protein. In certain embodiments, fragments are about five to 500 amino acids long. For example, fragments may be at least 5, 6, 8, 10, 14, 20, 50, 70, 100, 110, 150, 200, 250, 300, 350, 400 or 450 amino acids long.

A “recombinant protein”, including a recombinant TTR protein, is a protein made using recombinant techniques, i.e., through the expression of a recombinant nucleic acid as described herein. Methods and techniques for the production of recombinant proteins are well known in the art.

“Single-chain Fvs” (scFvs) are Fv molecules in which the heavy and light chain variable regions have been connected by a flexible linker to form a single polypeptide chain, which forms an antigen-binding region, scFvs are discussed in detail in International Patent Application Publication No. WO 88/01649 and U.S. Pat. Nos. 4,946,778 and 5,260,203.

A “solid tumor” refers to an abnormal growth or mass of tissue that usually does not contain cysts or liquid areas. Solid tumors may be benign (not cancerous) or malignant (cancerous). Different types of solid tumors are named for the type of cells that form them. Examples of solid tumors are sarcomas, carcinomas, and lymphomas. Leukemias (cancers of the blood) generally do not form solid tumors

An antigen binding protein “specifically binds” to an antigen when the antigen binding protein exhibits demonstrates little to no binding to molecules other than the antigen. An antigen binding protein that specifically binds an antigen may, however, cross-react with antigens from different species. Typically, an antigen binding protein specifically binds an antigen when the dissociation constant (K_{D}) is ≤10⁻⁷ M as measured via a surface plasma resonance technique (e.g., BIACore, GE-Healthcare Uppsala, Sweden). An antigen binding protein specifically binds an antigen with “high affinity” when it binds with a K_{D}≤5×10⁻⁸ M, and with “very high affinity” when it binds with a K_{D} is ≤5×10⁻⁹ M (as measured using a method such as BIACore).

A “subject” or “patient” as used herein can be any mammal. In a typical embodiment, the subject or patient is a human.

As used herein, “substantially pure” means that the described species of molecule is the predominant species present, that is, on a molar basis it is more abundant than any other individual species in the same mixture. In certain embodiments, a substantially pure molecule is a composition wherein the object species comprises at least 50% (on a molar basis) of all macromolecular species present. In other embodiments, a substantially pure composition will comprise at least 80%, 85%, 90%, 95%, or 99% of all macromolecular species present in the composition. In other embodiments, the object species is purified to essential homogeneity wherein contaminating species cannot be detected in the composition by conventional detection methods and thus the composition consists of a single detectable macromolecular species.

The term “treating” refers to any indication of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, certain methods presented herein successfully treat cancer and tumors, by, for instance, decreasing the progression or spreading of the cancer, inhibiting tumor growth, causing remission of the tumor and/or ameliorating a symptom associated with the cancer or tumor. Likewise, other methods provided herein treat infectious disease by decreasing the progression or spread of the infection, reducing the extent of the infection and/or ameliorating a symptom associated with the infection.

As used herein, the term “TTR,” refers to “transthyretin.” Human TTR is described in Mita et al., Biochem. Biophys. Res. Commun., 124(2):558-564 (1984), which is incorporated herein by reference. The amino acid sequence for human TTR is also described in the UniProt Knowledgebase (www.uniprot.org/uniprot/P02766#sequences) and is recited herein as SEQ ID NO: 1. The nucleic acid sequence for human TTR is described at NCBI (www.ncbi.nlm.nih.gov/gene/7276). See also GenBank deposit K02091.1. The nucleic acid sequence for human TTR is recited herein as SEQ ID NO: 44. The amino acid and nucleic acid sequences of murine TTR are set forth in SEQ ID NOs: 2 and 3, respectively. In some embodiments, the human TTR nucleic acid is a nucleic acid that encodes the human TTR protein of SEQ ID NO: 1. In other embodiments, the murine TTR nucleic acid is a nucleic acid that encodes the murine TTR protein of SEQ ID NO: 2.

The term “TTR variant” refers to a protein having an amino acid sequence which is at least 80%, 85%, 90%, 91%, 92%, 93%, 94%, 95%, 96%, 97%, 98%, or 99% identical to TTR having SEQ ID NO: 1. The present invention also includes nucleic acids encoding such TTR variants. Specific variants include, for example, TTR proteins with truncations at the C- or N-terminus.

A “tumor” refers to the mass of tissue formed as cancerous cells grow and multiply, which can invade and destroy normal adjacent tissues. Cancer cells can break away from a malignant tumor and enter the bloodstream or lymphatic system, such that cancer cells spread from the primary tumor to form new tumors in other organs.

A “variant” of a polypeptide comprises an amino acid sequence wherein one or more amino acid residues are inserted into, deleted from and/or substituted into the amino acid sequence relative to another polypeptide sequence. Variants include fusion proteins.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the present invention is intended to include such other forms of expression vectors, such as viral vectors (e.g., replication defective retroviruses, adenoviruses and adeno-associated viruses), which serve equivalent functions.

### TTR Variants

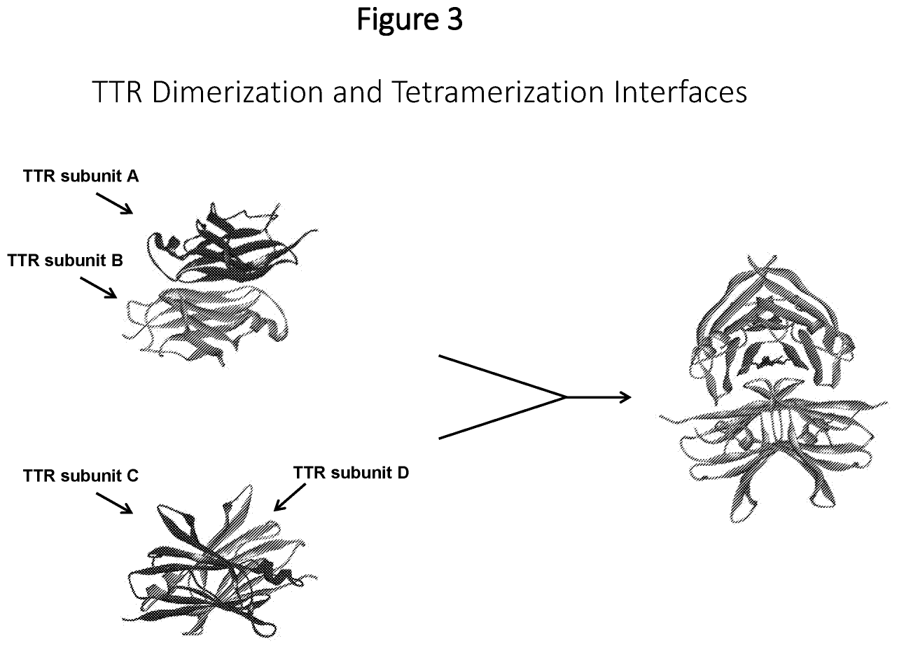

As previously discussed, human TTR is a non-covalent tetrameric protein. The TTR tetrameric protein is comprised of a dimer of dimers (FIG. 3). Interestingly, the interface between the TTR monomers which form TTR dimers (FIG. 3, left side) and the interface between the TTR dimers which form TTR tetramers (FIG. 3, right side) differs. The differences between the two interfaces allows for the engineering of TTR variants which modulate the interaction between the TTR dimers without disrupting the interface between the TTR monomers.

In one aspect of the present invention, each of the four TTR monomers which make up the tetrameric protein can be described as TTR subunit A, B, C, or D—wherein TTR subunits A and B form a first AB dimer and TTR subunits C and D form a second CD dimer (FIG. 3). TTR dimer AB and TTR dimer CD associate to form TTR tetramer ABCD. The TTR monomers of the present invention comprise at least one amino acid mutation (with respect to SEQ ID NO: 1) in the interface between TTR dimer AB and TTR dimer CD such that the formation of an ABCD tetramer is favored over the formation of any other tetramer (e.g., an ABAB tetramer or a CDCD tetramer.

Accordingly, the present invention relates to a TTR protein complex, wherein
the TTR protein complex comprises TTR subunits A, B, C, and D;
TTR subunits A and B dimerize to form TTR dimer AB;
TTR subunits C and D dimerize to form TTR dimer CD;
TTR dimers AB and CD further dimerize to form TTR tetramer ABCD; and
each of A, B, C, and D comprise the amino acid sequence of SEQ ID NO: 1, except that at least one amino acid in the interface between TTR dimer AB and TTR dimer CD is mutated such that the formation of an ABCD tetramer is favored over the formation of any other tetramer (e.g., an ABAB tetramer or a CDCD tetramer.

Each of A, B, C, and D of the TTR protein complex may comprise the amino acid sequence of SEQ ID NO: 1 with the following mutations: C10A, K15A, or both C10A and K15A.

Thus, in one embodiment, the present invention relates to TTR protein complexes wherein both A and B, both C and D, or all four of A, B, C, and D comprise a mutation at one or more amino acids positions selected from the list comprising: 6, 7, 8, 9, 10, 13, 15, 17, 19, 20, 21, 22, 23, 24, 26, 50, 51, 52, 53, 54, 56, 57, 60, 61, 62, 63, 78, 82, 83, 84, 85, 100, 101, 102, 103, 104, 106, 108, 110, 112, 113, 114, 115, 117, 119, 121, 123, 124, 125, 126, and 127 of SEQ ID NO: 1. In some embodiments, the mutations are in addition to C10A and K15A.

In another embodiment, the present invention relates to a TTR protein complex wherein both A and B, both C and D, or all four of A, B, C, and D comprise a mutation at one or more amino acids positions selected from the list comprising: 15, 17, 20, 21, 22, 23, 24, 51, 52, 84, 106, 108, 112, 114, 115, 119, 121, and 123 of SEQ ID NO: 1. In some embodiments, the mutations are in addition to C10A and K15A.

In another embodiment, the present invention relates to a TTR protein complex wherein both A and B, both C and D, or all four of A, B, C, and D comprise a mutation at one or more amino acids positions selected from the list comprising: 15, 17, 20, 21, 22, 23, 24, 51, 52, 84, 106, 108, 112, 114, 115, 119, 121, and 123 of SEQ ID NO: 1, wherein said amino acid is mutated to an aspartate, glutamate, arginine, lysine, or histidine. In some embodiments, the mutations are in addition to C10A and K15A.

In another embodiment, the present invention relates to a TTR protein complex wherein A and B comprise a mutation at one or more amino acids positions selected from the list comprising: 15, 17, 20, 21, 22, 23, 24, 51, 52, 84, 106, 108, 112, 114, 115, 119, 121, and 123 of SEQ ID NO: 1, wherein said amino acid is mutated to an aspartate or glutamate. In some embodiments, the mutations are in addition to C10A and K15A.

In yet another embodiment, the present invention relates to a TTR protein complex wherein C and D comprise a mutation at one or more amino acids positions selected from the list comprising: 15, 17, 20, 21, 22, 23, 24, 51, 52, 84, 106, 108, 112, 114, 115, 119, 121, and 123 of SEQ ID NO: 1, wherein said amino acid is mutated to an arginine, lysine, or histidine. In some embodiments, the mutations are in addition to C10A and K15A.

In a particular embodiment, A and B comprise a mutation at one or more amino acids positions selected from the list comprising: 15, 17, 20, 21, 22, 23, 24, 51, 52, 84, 106, 108, 112, 114, 115, 119, 121, and 123 of SEQ ID NO: 1, wherein said amino acid is mutated to an aspartate or glutamate; and C and D comprise a mutation at one or more amino acids positions selected from the list comprising: 15, 17, 20, 21, 22, 23, 24, 51, 52, 84, 106, 108, 112, 114, 115, 119, 121, and 123 of SEQ ID NO: 1, wherein said amino acid is mutated to an arginine, lysine, or histidine. In some embodiments, the mutations are in addition to C10A and K15A.

In some embodiments, A and B comprise at least one mutation in SEQ ID NO: 1, wherein said mutation is selected from the list comprising: K15D, L17D, V20D, R21D, G22D, S23D, P24D, S52D, 184D, T106D, A108D, S112D, Y114D, S115D, T119D, V121D, S123D, K15E, L17E, V20E, R21E, G22E, S23E, P24E, D51E, S52E, 184E, T106E, A108E, S112E, Y114E, S115E, T119E, V121E, and S123E. The present invention also relates to TTR protein complexes wherein A and B comprise at least one mutation in SEQ ID NO: 1, wherein the mutation is selected from the list comprising: L17D, L17E, V20D, V20E, G22D, G22E, S112D, S112E, T119D, T119E, V121D, and V121E. In some embodiments, the mutations are in addition to C10A and K15A.

In some embodiments, C and D comprise at least one mutation in SEQ ID NO: 1, wherein said mutation is selected from the list comprising: K15R, L17R, V20R, G22R, S23R, P24R, D51R, S52R, 184R, T106R, A108R, S112R, Y114R, S115R, T119R, V121R, S123R, L17K, V20K, R21K, G22K, S23K, P24K, D51K, S52K, 184K, T106K, A108K, S112K, Y114K, S115K, T119K, V121K, S123K, K15H, L17H, V20H, R21H, G22H, S23H, P24H, D51H, S52H, 184H, T106H, A108H, S112H, Y114H, S115H, T119H, V121H, and S123H. The present invention also relates to TTR protein complexes wherein C and D comprise at least one mutation in SEQ ID NO: 1, wherein the mutation is selected from the list comprising: L17R, L17K, L17H, V20R, V20K, V20H, G22R, G22K, G22H, S112R, S112K, S112H, T119R, T119K, T119H, V121R, V121K, and V121H. In some embodiments, the mutations are in addition to C10A and K15A.

The TTR protein complex of the present invention can comprise TTR subunits wherein both A and B, both C and D, or all four of A, B, C, and D independently comprise one or two mutations discussed herein. In some embodiments, the TTR protein complex of the present invention can comprise TTR subunits wherein both A and B, both C and D, or all four of A, B, C, and D independently comprise one mutation discussed herein. In some embodiments, the mutations are in addition to C10A and K15A.

In specific embodiments, TTR protein complexes of the present invention comprise TTR subunits wherein each of A, B, C, and D comprise the amino acid sequence of SEQ ID NO: 1 with the following mutations (and vice versa) in Table 1:

TABLE 1

| Mutation in TTR | Mutation in TTR | Mutation in TTR | Mutation in TTR |
|---|---|---|---|
| Subunit A and B* | Subunit C and D* | Subunit A and B* | Subunit C and D* |
| C10A/K15A/L17D | C10A/K15A/L17R | C10A/K15A/L17D | C10A/K15A/S112R |
| C10A/K15A/L17E | C10A/K15A/L17R | C10A/K15A/L17E | C10A/K15A/S112R |
| C10A/K15A/V20D | C10A/K15A/L17R | C10A/K15A/V20D | C10A/K15A/S112R |
| C10A/K15A/V20E | C10A/K15A/L17R | C10A/K15A/V20E | C10A/K15A/S112R |
| C10A/K15A/G22D | C10A/K15A/L17R | C10A/K15A/G22D | C10A/K15A/S112R |
| C10A/K15A/G22E | C10A/K15A/L17R | C10A/K15A/G22E | C10A/K15A/S112R |
| C10A/K15A/S112D | C10A/K15A/L17R | C10A/K15A/S112D | C10A/K15A/S112R |
| C10A/K15A/S112E | C10A/K15A/L17R | C10A/K15A/S112E | C10A/K15A/S112R |
| C10A/K15A/T119D | C10A/K15A/L17R | C10A/K15A/T119D | C10A/K15A/S112R |
| C10A/K15A/T119E | C10A/K15A/L17R | C10A/K15A/T119E | C10A/K15A/S112R |
| C10A/K15A/V121D | C10A/K15A/L17R | C10A/K15A/V121D | C10A/K15A/S112R |
| C10A/K15A/V121E | C10A/K15A/L17R | C10A/K15A/V121E | C10A/K15A/S112R |
| C10A/K15A/L17D | C10A/K15A/L17K | C10A/K15A/L17D | C10A/K15A/S112K |
| C10A/K15A/L17E | C10A/K15A/L17K | C10A/K15A/L17E | C10A/K15A/S112K |
| C10A/K15A/V20D | C10A/K15A/L17K | C10A/K15A/V20D | C10A/K15A/S112K |
| C10A/K15A/V20E | C10A/K15A/L17K | C10A/K15A/V20E | C10A/K15A/S112K |
| C10A/K15A/G22D | C10A/K15A/L17K | C10A/K15A/G22D | C10A/K15A/S112K |
| C10A/K15A/G22E | C10A/K15A/L17K | C10A/K15A/G22E | C10A/K15A/S112K |
| C10A/K15A/S112D | C10A/K15A/L17K | C10A/K15A/S112D | C10A/K15A/S112K |
| C10A/K15A/S112E | C10A/K15A/L17K | C10A/K15A/S112E | C10A/K15A/S112K |
| C10A/K15A/T119D | C10A/K15A/L17K | C10A/K15A/T119D | C10A/K15A/S112K |
| C10A/K15A/T119E | C10A/K15A/L17K | C10A/K15A/T119E | C10A/K15A/S112K |
| C10A/K15A/V121D | C10A/K15A/L17K | C10A/K15A/V121D | C10A/K15A/S112K |
| C10A/K15A/V121E | C10A/K15A/L17K | C10A/K15A/V121E | C10A/K15A/S112K |
| C10A/K15A/L17D | C10A/K15A/V20R | C10A/K15A/L17D | C10A/K15A/T119R |
| C10A/K15A/L17E | C10A/K15A/V20R | C10A/K15A/L17E | C10A/K15A/T119R |
| C10A/K15A/V20D | C10A/K15A/V20R | C10A/K15A/V20D | C10A/K15A/T119R |
| C10A/K15A/V20E | C10A/K15A/V20R | C10A/K15A/V20E | C10A/K15A/T119R |
| C10A/K15A/G22D | C10A/K15A/V20R | C10A/K15A/G22D | C10A/K15A/T119R |
| C10A/K15A/G22E | C10A/K15A/V20R | C10A/K15A/G22E | C10A/K15A/T119R |
| C10A/K15A/S112D | C10A/K15A/V20R | C10A/K15A/S112D | C10A/K15A/T119R |
| C10A/K15A/S112E | C10A/K15A/V20R | C10A/K15A/S112E | C10A/K15A/T119R |
| C10A/K15A/T119D | C10A/K15A/V20R | C10A/K15A/T119D | C10A/K15A/T119R |
| C10A/K15A/T119E | C10A/K15A/V20R | C10A/K15A/T119E | C10A/K15A/T119R |
| C10A/K15A/V121D | C10A/K15A/V20R | C10A/K15A/V121D | C10A/K15A/T119R |
| C10A/K15A/V121E | C10A/K15A/V20R | C10A/K15A/V121E | C10A/K15A/T119R |
| C10A/K15A/L17D | C10A/K15A/A20K | C10A/K15A/L17D | C10A/K15A/T119K |
| C10A/K15A/L17E | C10A/K15A/V20K | C10A/K15A/L17E | C10A/K15A/T119K |
| C10A/K15A/V20D | C10A/K15A/V20K | C10A/K15A/V20D | C10A/K15A/T119K |
| C10A/K15A/V20E | C10A/K15A/V20K | C10A/K15A/V20E | C10A/K15A/T119K |
| C10A/K15A/G22D | C10A/K15A/V20K | C10A/K15A/G22D | C10A/K15A/T119K |
| C10A/K15A/G22E | C10A/K15A/V20K | C10A/K15A/G22E | C10A/K15A/T119K |
| C10A/K15A/S112D | C10A/K15A/V20K | C10A/K15A/S112D | C10A/K15A/T119K |
| C10A/K15A/S112E | C10A/K15A/V20K | C10A/K15A/S112E | C10A/K15A/T119K |
| C10A/K15A/T119D | C10A/K15A/V20K | C10A/K15A/T119D | C10A/K15A/T119K |
| C10A/K15A/T119E | C10A/K15A/V20K | C10A/K15A/T119E | C10A/K15A/T119K |
| C10A/K15A/V121D | C10A/K15A/V20K | C10A/K15A/V121D | C10A/K15A/T119K |
| C10A/K15A/V121E | C10A/K15A/V20K | C10A/K15A/V121E | C10A/K15A/T119K |
| C10A/K15A/L17D | C10A/K15A/G22R | C10A/K15A/L17D | C10A/K15A/V121R |
| C10A/K15A/L17E | C10A/K15A/G22R | C10A/K15A/L17E | C10A/K15A/V121R |
| C10A/K15A/V20D | C10A/K15A/G22R | C10A/K15A/V20D | C10A/K15A/V121R |
| C10A/K15A/V20E | C10A/K15A/G22R | C10A/K15A/V20E | C10A/K15A/V121R |
| C10A/K15A/G22D | C10A/K15A/G22R | C10A/K15A/G22D | C10A/K15A/V121R |
| C10A/K15A/G22E | C10A/K15A/G22R | C10A/K15A/G22E | C10A/K15A/V121R |
| C10A/K15A/S112D | C10A/K15A/G22R | C10A/K15A/S112D | C10A/K15A/V121R |
| C10A/K15A/S112E | C10A/K15A/G22R | C10A/K15A/S112E | C10A/K15A/V121R |
| C10A/K15A/T119D | C10A/K15A/G22R | C10A/K15A/T119D | C10A/K15A/V121R |
| C10A/K15A/T119E | C10A/K15A/G22R | C10A/K15A/T119E | C10A/K15A/V121R |
| C10A/K15A/V121D | C10A/K15A/G22R | C10A/K15A/V121D | C10A/K15A/V121R |
| C10A/KI5A/V121E | C10A/K15A/G22R | C10A/K15A/V121E | C10A/K15A/V121R |
| C10A/K15A/L17D | C10A/K15A/G22K | C10A/K15A/L17D | C10A/K15A/V121K |
| C10A/K15A/L17E | C10A/K15A/G22K | C10A/K15A/L17E | C10A/K15A/V121K |
| C10A/K15A/V20D | C10A/K15A/G22K | C10A/K15A/V20D | C10A/K15A/V121K |
| C10A/K15A/V20E | C10A/K15A/G22K | C10A/K15A/V20E | C10A/K15A/V121K |
| C10A/K15A/G22D | C10A/K15A/G22K | C10A/K15A/G22D | C10A/K15A/V121K |
| C10A/K15A/G22E | C10A/K15A/G22K | C10A/K15A/G22E | C10A/K15A/V121K |
| CI0A/K15A/S112D | C10A/K15A/G22K | C10A/K15A/S112D | C10A/K15A/V121K |
| C10A/K15A/S112E | C10A/K15A/G22K | C10A/K15A/S112E | C10A/K15A/V121K |
| C10A/K15A/T119D | C10A/K15A/G22K | C10A/K15A/T119D | C10A/K15A/V121K |
| C10A/K15A/T119E | C10A/K15A/G22K | C10A/K15A/T119E | C10A/K15A/V121K |
| C10A/K15A/V121D | C10A/K15A/G22K | C10A/K15A/V121D | C10A/K15A/V121K |
| C10A/K15A/V121E | C10A/K15A/G22K | C10A/K15A/V121E | C10A/K15A/V121K |

*Relative to SEQ ID NO: 1

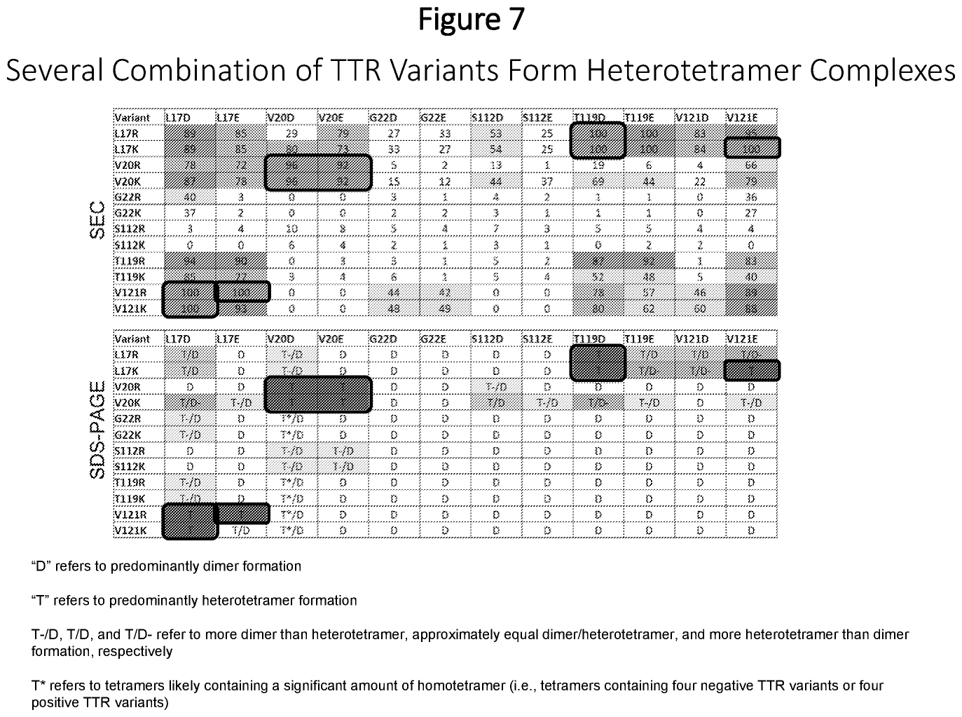

Any of the TTR variants and variant pairings in Table 1 are suitable for use in in the present invention. Table 2 notes the amount of TTR tetramer formation observed for certain variants and pairing (see Example 2 and FIG. 7).

TABLE 2
TTR Tetramer Formation

| Relative Amount of | | |
|---|---|---|
| Tetramer Formation* | Negative | Positive |
| 1 | L17D | V121R |
| 1 | L17D | V121K |
| 1 | L17E | V121R |
| 1 | V20D | V20R |
| 1 | V20D | V20K |
| 1 | V20E | V20R |
| 1 | V20E | V20K |
| 1 | T119D | L17R |
| 1 | T119D | L17K |
| 1 | V121E | L17K |
| 2 | L17D | L17R |
| 2 | L17D | L17K |
| 2 | L17D | V20R |
| 2 | L17D | V20K |
| 2 | L17D | T119R |
| 2 | L17D | T119K |
| 2 | L17E | L17R |
| 2 | L17E | L17K |
| 2 | L17E | V20R |
| 2 | L17E | V20K |
| 2 | L17E | T119R |
| 2 | L17E | T119K |
| 2 | L17E | V121K |
| 2 | V20D | L17K |
| 2 | V20E | L17R |
| 2 | V20E | L17K |
| 2 | T119D | V20K |
| 2 | T119D | T119R |
| 2 | T119D | V121R |
| 2 | T119D | V121K |
| 2 | T119E | L17R |
| 2 | T119E | L17K |
| 2 | T119E | T119R |
| 2 | T119E | V121K |
| 2 | V121D | L17R |
| 2 | V121D | L17K |
| 2 | V121E | L17R |
| 2 | V121E | V20R |
| 2 | V121E | V20K |
| 2 | V121E | T119R |
| 2 | V121E | V121R |
| 2 | V121E | V121K |
| 3 | T119D | T119K |
| 3 | I119E | V121R |
| 3 | L17D | G22R |
| 3 | L17D | G22K |
| 3 | V20D | L17R |
| 3 | G22D | L17R |
| 3 | G22D | L17K |
| 3 | G22D | V20K |
| 3 | G22D | V121R |
| 3 | G22D | V121K |
| 3 | G22E | L17R |
| 3 | G22E | L17K |
| 3 | G22E | V20K |
| 3 | G22E | V121R |
| 3 | G22E | V121K |
| 3 | S112D | L17R |
| 3 | S112D | L17K |
| 3 | S112D | V20R |
| 3 | S112D | V20K |
| 3 | S112E | L17R |
| 3 | S112E | L17K |
| 3 | S112E | V20K |
| 3 | T119D | V20R |
| 3 | T119E | V20K |
| 3 | T119E | T119K |
| 3 | V121D | V20K |
| 3 | V121D | V121R |
| 3 | V121D | V121K |
| 3 | V121E | G22R |
| 3 | V121E | G22K |
| 3 | V121E | T119K |

*Rank 1 = strong tetramers by SEC and SDS resistant
Rank 2 = strong tetramers by SEC >60%, marginally or not SDS resistant
Rank 3 = significant tetramers by SEC >10% and <=60%

The TTR protein complex of the present invention can comprise TTR subunits wherein both A and B, both C and D, or all four of A, B, C, and D independently comprise two mutations discussed herein. In some embodiments, A and B comprise two mutations in SEQ ID NO: 1, wherein said mutations are selected from the list comprising: L117D/V20D, L117D/V20E, L17E/V20D. L17E/V20E, L117D/T119D, L17D/V121E. L17E/T119D, L17E/V121E, V20D/T119D, V20D/V121E, V20E/T119D, and V20E/V121E. In some embodiments, the mutations are in addition to C10A and K15A.

In some embodiments, C and D comprise two mutations in SEQ ID NO: 1, wherein said mutations are selected from the list comprising: L17K/V20K, L17K/V20R, L17R/V20K, L17R/V20R, L17K/V121K, L17K/V121R, L17R/V121K, L17R/V121R, V20K/V121K, V20K/V121R, V20R/V121K, and V20R/V121R. In some embodiments, the mutations are in addition to C10A and K15A.

In specific embodiments, TTR protein complexes of the present invention comprise TTR subunits wherein each of A, B, C, and D comprise the amino acid sequence of SEQ ID NO: 1 with the following mutations (and vice versa) in Table 3:

TABLE 3

| Mutations in TTR | Mutations in TTR | Mutations in TTR | Mutations in TTR |
|---|---|---|---|
| Subunit A and B* | Subunit C and D* | Subunit A and B* | Subunit C and D* |
| C10A/K15A/L17D/V20D | C10A/K15A/L17K/V20K | C10A/K15A/L17E/T119D | C10A/K15A/L17R/V121K |
| C10A/K15A/L17D/V20E | C10A/K15A/L17K/V20R | C10A/K15A/L17E/V121E | C10A/K15A/L17R/V121R |
| C10A/K15A/L17E/V20D | C10A/K15A/L17R/V20K | C10A/K15A/V20D/T119D | C10A/K15A/V20K/V121K |
| C10A/K15A/L17E/V20E | C10A/K15A/L17R/V20R | C10A/K15A/V20D/V121E | C10A/K15A/V20K/V121R |
| C10A/K15A/L17D/T119D | C10A/K15A/L17K/V121K | C10A/K15A/V20E/T119D | C10A/K15A/V20R/V121K |
| C10A/K15A/L17D/V121E | C10A/K15A/L17K/V121R | C10A/K15A/V20E/V121E | C10A/K15A/V20R/V121R |

*Relative to SEQ ID NO: 1

In some embodiments, the TTR protein complexes of the present invention comprise TTR subunits wherein A and B, or C and D, comprise the amino acid sequence of SEQ ID NO: 1 with the following mutations C10A/K15A/V20E/T119D, C10A/K15A/L17D/T119D, C10A/K15A/L17E/T119D, C10A/K15A/L17R/V20K, C10A/K15A/L17K/V20K, C10A/K15A/L17R/V121R or C10A/K15A/L17R/V121K.

As discussed above. TTR variants may also be used in the present invention. Any of the TTR variants discussed herein may be utilized in combination with each other. TTR variants include proteins having an amino acid sequence which is at least 80%, 81%, 82%, 83%, 86%, 85%, 86%, 87%, 88%, 89%, 90%, 91%, 92%, 93%, 94%, 95%, 96%, 97%, 98%, or 99% identical to a TTR protein having a mutation with respect to SEQ ID NO: 1.

Cysteines present in human TTR (SEQ ID NO: 1) may be used as sites of conjugation to bioactive proteins, peptides, or small molecules. In some embodiments, the cysteines present in human TTR (SEQ ID NO: 1) may be used as sites of conjugation to antigen binding proteins (e.g., antibodies and Fabs). In addition, TTR variants that enable site specific conjugation, such as TTR variants with engineered cysteines, may be used in the present invention. See, e.g., U.S. Pat. No. 8,633,153, which is hereby incorporated by reference. For example, a TTR variant may include one or more of the following cysteine mutations:

### A37C, D38C, A81C, or G83C.

Additional variants useful in the present invention include, for example, TTR proteins with truncations at the C- or N-terminus. Such TTR proteins include those wherein 1, 2, 3, 4, 5, 6, 7, 8, 9, 10, 11, 12, 13, 14, 15, 16, 17, 18, 19, or 20 amino acids are removed from the C- or N-terminus TTR protein. In some embodiments, the fusion proteins of the present invention comprise TTR proteins wherein 1, 2, 3, 4, 5, 6, 7, or 8 amino acids are removed from the C- or N-terminus of the TTR protein. In other embodiments, the fusion proteins of the present invention comprise TTR proteins wherein 1, 2, 3, 4, 5, 6, 7, or 8 amino acids are removed from the N-terminus of the TTR protein.

Additional TTR variants that can be used in the present invention include those which reduce or block TTR binding to thyroxine. Each TTR tetramer contains two thyroxine binding sites located in the central channel of the TTR tetramer. Such variants, for example, could avoid interference with thyroxine biology in patients and may avoid having TTR fusions acted upon by the thyroxine metabolism path. Yet other TTR variants that can be used in the present invention include those that reduce or eliminate the proteolytic activity of TTR.

In addition, TTR-His tag fusions may be used in the present invention. For example, TTR-His tag fusions may be used in the purification of TTR Fab constructs wherein the Fab lacks an Fc, or for the purification of TTR Ab constructs where it is beneficial to avoid the low pH purification environment of a Protein A affinity column. In some embodiments, the His tag is removed after purification. His tags may also be present in the final therapeutic molecule (i.e., the tag may be retained after purification). In some embodiments, the His tag is a His, (His)₂, (His)₃, (His)₄, (His)₅, (His)₆, (His)₇, (His)₈, (His)₉, or (His)₁₀ tag. In particular embodiments, the His tag is a (His)₆ or (His), tag. In a specific embodiment, the His tag is a (His)₆ tag. In some embodiments, the His tag includes 1, 2, 3, 4, 5, 6, 7, 8, 9, or 10 glycine amino acids as a linker. In a particular embodiment, the His tag includes two glycines (e.g., GGHHHHHH).

In some embodiments, a two glycine amino acid linker can be inserted between the TTR variant and the heavy or light chain.

Moreover, the TTR variant of the present invention may include variants incorporating glycosylation sites which may be helpful in modulating the PK or solubility properties of the TTR fusions. In addition, the TTR variants or TTR fusion proteins of the present invention may modified to include moieties which confer beneficial PK properties, e.g., triazine-containing moieties (contained within constructs having a terminal group capable of reacting with a protein; see, for example PCT publication No. WO/2017/083604 which is hereby incorporated by reference in its entirety).

In some embodiments, the TTR protein complex is attached to 1, 2, 3, 4, 5, 6, 7, or 8 antigen binding proteins or peptides. In other embodiments, the TTR protein complex is attached to 1, 2, 3, or 4 antigen binding proteins or peptides. The antigen binding proteins or peptides may be attached to the TTR protein complex at the C-terminus of a TTR subunit or the N-terminus of a TTR subunit. In addition, the TTR protein complex may be directly attached to 1, 2, 3, 4, 5, 6, 7, or 8 antigen binding proteins or peptides; or may be attached to 1, 2, 3, 4, 5, 6, 7, or 8 antigen binding proteins or peptides via a linker. In particular embodiments, the TTR protein complex is directly attached to 1, 2, 3, or 4 antigen binding proteins or peptides; or is attached to 1, 2, 3, or 4 antigen binding proteins via a linker or peptides.

### Heterodimer Fusion Proteins (Complexes)

As described herein, the present invention relates in part to the use of TTR in the multimerization of antigen binding proteins, such as antibodies. Because TTR is a human extracellular protein found in human serum, it is present in relatively high amounts throughout the human body, making it less likely to elicit an immune response when present in the multimerization constructs of the present invention (compared to, e.g., non-human, intracellular and rare proteins). Accordingly, its use in in the multimerization techniques of the present invention is advantageous.

For example, TTR can be used in the dimerization of antibodies that bind different epitopes, wherein the epitopes are present e.g., on the same or different proteins. In such heterodimer fusion proteins, TTR (SEQ ID NO: 1), or a variant thereof, is present as a tetramer wherein a TTR subunit is linked to the C-terminus of an antibody heavy chain to form TTR antibody heterodimers. For example, the C-terminus of each antibody heavy chain (with each antibody containing two such C-termini) may be linked to the N-terminus of each TTR subunit (see FIGS. 1 and 2). Thus, each antibody is linked to two TTR subunits in the TTR tetramer, yielding a TTR antibody heterodimer.

Accordingly, the present invention relates to heterodimer fusion proteins comprising two antigen binding proteins, wherein each antigen binding protein binds a different epitope, wherein the epitopes are present e.g., on the same or different proteins. In some embodiments, the heterodimer fusion proteins comprise antigen binding proteins linked to a protein complex. In some embodiments, the protein complex is a TTR protein complex, wherein the TTR protein complex is a TTR tetramer. In some embodiments the antigen binding protein is an antibody.

In particular embodiments, the present invention relates to heterodimer fusion proteins comprising two antibodies linked to a TTR tetramer, wherein each antigen binding protein binds a different epitope, wherein the epitopes are present e.g., on the same or different proteins. The antibodies may be connected to the TTR tetramer without a linker (i.e., the antibodies are directly connected to the TTR).

In other embodiments, the antibodies are connected to the TTR tetramer via a linker. For example, amino acid linkers may be used to link the C-terminus of the antibody heavy chain to the TTR subunit N-terminus. In some embodiments, the linker is 1-5, 1-10, 1-15, 1-20, 1-25, 1-30, 1-35, or 1-40 amino acids in length. In some embodiments, the linker is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids in length. In other embodiments, the linker is 0, 1, 5, 10, 15, 20, 25, 30, 35, or 40 amino acids in length. In other embodiments, the linker is up to 5, 10, 15, 20, 25, 30, 35, or 40 amino acids in length. In some embodiments, the linker is up to 5, 10, 15, or 20 amino acids in length. In particular embodiments, the linker is 0, 5, 10, 15, or 20 amino acids in length.

In some embodiments, the linker is GGGGS, GGGGSGGGGS (i.e., (GGGGS)₂), GGGGSGGGGSGGGGS (i.e., (GGGGS)₃), GGGGSGGGGSGGGGSGGGGS (i.e., (GGGGS)₄), GGGGSGGGGSGGGGSGGGGSGGGGS (i.e., (GGGGS)₅), or GGGGSGGGGSGGGGSGGGGSGGGGSGGGGS (i.e., (GGGGS)₆). In other embodiments, the is GGGGS, GGGGSGGGGS (i.e., (GGGGS)₂), GGGGSGGGGSGGGGS (i.e., (GGGGS)₃), or GGGGSGGGGSGGGGSGGGGS (i.e., (GGGGS)₄).

Other suitable amino acid linkers include, for example, disulfide bonds, (Gly)ₙ (n=1-10), (EAAAK)ₙ (n=1-5), A(EAAAK)₄ALEA(EAAAK)₄A, PAPAP, AEAAAKEAAAKA, (Ala-Pro)ₙ (n=1-20), VSQTSKLTRAETVFPDV, PLGLWA, RVLAEA, EDVVCCSMSY, GGIEGRGS, TRHRQPRGWE, AGNRVRRSVG, RRRRRRRRR, GFLG, and LE. Suitable non-amino acid linkers include polyethylene glycol (PEG).

In some embodiments, the antibodies are connected to a truncated TTR subunit, with or without a linker. For example, 1, 2, 3, 4, 5, 6, 7, 8, 9, or 10 amino acids may be removed from the N-terminus of one or more TTR subunits, and the antibody may be attached to the truncated TTR subunit N-terminus.

The present invention also relates to nucleic acid molecules encoding the heterodimer fusion proteins described herein. Details regarding exemplary methods for producing the heterodimer fusion proteins can be found in the Examples.

### Heterotrimer and Heterotetramer Fusion Proteins (Complexes)

The present invention also relates in part to the use of TTR in the trimerization or tetramerization of antigen binding proteins, such as antibodies.

In heterotetramer fusion proteins, TTR (SEQ ID NO: 1), or a variant thereof, is again present as a tetramer. However, in the context of the TTR antibody heterotetramers, a single antibody heavy chain (i.e., only one of the two heavy chains present in a single antibody) is linked to each TTR subunit, allowing for the linkage of four antibodies to the TTR tetramer (see FIG. 2e). One of the two heavy chains of the antibody C-terminus may be linked to the N-terminus of each TTR subunit (see FIG. 2c). Thus, each antibody is linked to one TTR subunit in the TTR tetramer, yielding a TTR antibody heterotetramer.

In such heterotetramer fusion proteins, the formation of Fc heterodimers (discussed above) is disfavored through mutations in the Fc. Such modifications include Fc mutations such as knobs-into-holes, DuoBodies, Azymetric, charge pair, HA-TF, SEEDbody, and modifications with differential protein A affinity. See, e.g., Spiess et al., Molecular Immunology, 67(2, Part A), 2015, pp. 95-106. Knobs-into-holes mutations include T366W in the first heavy chain, and T366S, L368A, and/or Y407V in the second heavy chain. See, e.g., Ridgway et al., Protein Eng., 9 (1996), pp. 617-621; and Atwell et al., J. Mol. Biol., 270 (1997), pp. 26-35. DuoBody mutations include F405L in the first heavy chain and K409R in the second heavy chain. See, e.g., Labrijn et al., Proc. Natl. Acad. Sci. U.S.A., 110 (2013), pp. 5145-5150. Azymetric mutations include T350V, L351Y, F405A, and/or Y407V in the first heavy chain, and T350V, T366L, K392L, and/or T394W in the second heavy chain. See, e.g., Von Kreudenstein et al., mAbs, 5 (2013), pp. 646-654. HA-TF mutations include S364H and/or F405A in the first heavy chain, and Y349T and/or T394F in the second heavy chain. See, e.g., Moore et al., mAbs, 3 (2011), pp. 546-557. SEEDbody mutations include IgG/A chimera mutations in the first heavy chain and IgG/A chimera mutations in the second heavy chain. See, e.g., Davis et al., Protein Eng. Des. Sel., 23 (2010), pp. 195-202. Differential protein A affinity mutations include H435R in one heavy chain and no mutations in the other heavy chain. See, e.g., U.S. Pat. No. 8,586,713. Each of these documents is incorporated by reference in its entirety.

In particular embodiments, it is possible to drive heterotetramerization of the antibodies through the use of Fc charge pairs which disfavor dimerization of the antibody heavy chains, thus favoring heavy chain dimerization between one antibody heavy chain which is linked to a TTR subunit and one antibody heavy chain that is not linked to a TTR (see FIG. 1c). For example, a set of charged mutations may be incorporated in the C_{H}3 domain of the heavy chain with either negative charges on one heavy chain and positive charges on the corresponding heavy chain, or a mixture of negative and positive charges on one heavy chain which pair with their corresponding positive and negative charges on the corresponding heavy chain. Exemplary negative charges include K392D & K409D and exemplary positive charges include E356K & D399K. Since like charges at the C_{H}3 interface repel while dissimilar charges attract, homodimerization is disfavored while heterodimerization is favored. TTR is fused to the heavy chain of only one charge type (either positive or negative, but not both); thus, resulting in one TTR subunit per full antibody composed of 4 chains (two light chains, one unfused heavy chain and one TTR fused heavy chain). Additional charge pair mutations are discussed in, for example, U.S. Pat. No. 9,546,203. Charge pair mutations including D221E, P228E, and/or L368E in the first heavy chain and D221R, P228R, and/or K409R in the second heavy chain are also described in, e.g., Strop et al., J. Mol. Biol., 420 (2012), pp. 204-219. Each of these documents is incorporated by reference in its entirety.

In heterotrimer fusion proteins, TTR (SEQ ID NO: 1), or a variant thereof, is again present as a tetramer. Examples of heterotrimer fusion proteins include those comprising one antibody and two Fabs. In the context of the TTR antibody heterotrimers, the C-terminus of each antibody heavy chain may be linked to the N-terminus of each of two TTR subunits while the C-terminus of each of two Fabs is linked to the N-terminus of each of two TTR subunits to form a TTR Ab/Fab heterotrimer (see FIGS. 2c and 2d). Thus, the antibody is linked to two TTR subunits in the TTR tetramer and each Fab is linked to a TTR subunit yielding a TTR Ab/Fab heterotrimer comprising a TTR tetramer, one antibody, and two Fabs.

The present invention also relates in part to the use of TTR in the tetramerization of Fabs. In such heterotetramer fusion proteins, TTR (SEQ ID NO: 1), or a variant thereof, is again present as a tetramer wherein each TTR subunit is linked to the C-terminus of each Fab to form TTR Fab heterotetramers (see FIG. 2a). Thus, each Fab is linked to a single TTR subunit in the TTR tetramer, yielding a TTR Fab heterotetramer.

Accordingly, the present invention relates to heterotrimer and heterotetramer fusion proteins comprising three or four antigen binding proteins (e.g., an Ab/Fab trimer, a Fab tetramer, or an Ab tetramer). In some embodiments, the heterotrimer and heterotetramer fusion proteins comprise antigen binding proteins linked to a protein complex. In some embodiments, the protein complex is a TTR protein complex, wherein the TTR protein complex is a TTR tetramer. In some embodiments the antigen binding protein is an antibody. In other embodiments the antigen binding protein is a Fab. In some embodiments the heterotetramer fusion proteins comprise a mixture of antibodies and Fabs.

In particular embodiments, the present invention relates to heterotetramer fusion proteins comprising four antibodies linked to a TTR tetramer. In other embodiments, the present invention relates to heterotetramer fusion proteins comprising four Fabs linked via a linker to a TTR tetramer. In other embodiments, the present invention relates to heterotrimer fusion proteins comprising one Ab and two Fabs linked via a linker to a TTR tetramer. In some embodiments, the antibodies or Fabs are connected to the TTR tetramer without a linker (i.e., the antibodies or Fabs are directly connected to the TTR).

The linker may be an amino acid-based linker comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In other embodiments, the linker is an amino acid-based linker comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids. In other embodiments, the linker is an amino acid-based linker comprising 1, 2, 3, 4, 5, 6, 7, 8, 9, 10, 11, 12, 13, 14, 15, 16, 17, 18, 19, or 20 amino acids. In yet other embodiments, the linker is an amino acid-based linker comprising 2, 3, 4, 5, 6, 7, 8, 9, or 10 amino acids. In particular embodiments, the linker is G, GG, GGG, GGGG, GGGGG, GGGGGG, GGGGG GGGGGGGG, GGGGGGGGG, or GGGGGGGGGG. In other particular embodiments, the linker is selected from the list comprising: GG, GGGG, GGGSGG, GGGSGGGG, and GGAGGGAGGG.

In some embodiments, the linker is GGGGS, GGGGSGGGGS (i.e., (GGGGS)₂), GGGGSGGGGSGGGGS (i.e., (GGGGS)₃). GGGGSGGGGSGGGGSGGGGS (i.e., (GGGGS)₄), GGGGSGGGGSGGGGSGGGGSGGGGS (i.e., (GGGGS)₅), or GGGGSGGGGSGGGGSGGGGSGGGGSGGGGS (i.e., (GGGGS)₆). In other embodiments, the is GGGGS, GGGGSGGGGS (i.e., (GGGGS)₂), GGGGSGGGGSGGGGS (i.e., (GGGGS)₃) or GGGGSGGGGSGGGGSGGGGS (i.e., (GGGGS)₄).

Other suitable linkers include a G(GₓB_{y})ᵣG_{z} linker wherein G=glycine; B=any amino acid; x=1-15; y=1-5; z=1-15; and r=1-20. In another embodiment, the linker is a G(GₓB_{y})ᵣG_{z} linker wherein B=Q, S, A, E, P, T, K, R, D or N; x=4; =1; z=4; and r=1.

Additional suitable amino acid linkers include, for example, disulfide bonds, (Gly)ₙ (n=1-10), (EAAAK)ₙ (n=1-5), A(EAAAK)₄ALEA(EAAAK)₄A, PAPAP, AEAAAKEAAAKA, (Ala-Pro)ₙ (n=1-20), VSQTSKLTRAETVFPDV, PLGLWA, RVLAEA, EDVVCCSMSY, GGIEGRGS, TRHRQPRGWE, AGNRVRRSVG. RRRRRRRRR, GFLG, and LE. Suitable non-amino acid linkers include polyethylene glycol (PEG) and triazine-containing moieties (contained within constructs having a terminal group capable of reacting with a protein; see, for example PCT publication No. WO/2017/083604 which is hereby incorporated by reference in its entirety).

In some embodiments, the antibodies or Fabs are connected to a truncated TTR subunit, with or without a linker. For example, 1, 2, 3, 4, 5, 6, 7, 8, 9, or 10 amino acids may be removed from the N-terminus of one or more TTR subunits, and the antibodies or Fabs may be attached to the truncated TTR subunit N-terminus.

The present invention also relates to nucleic acid molecules encoding the heterotrimer and heterotetramer fusion proteins described herein. Details regarding exemplary methods for producing the heterotrimer and heterotetramer fusion proteins can be found in the Examples.

### Antigen Binding Proteins

Any antigen binding protein (e.g., Fab, antibody, scFv, scFab) can be used in the TTR fusion proteins of the present invention. In addition, proteins such as enzymes can be used in the TTR fusion proteins of the present invention in combination with antigen binding proteins.

Because the fusion proteins of the present invention allow for the binding of different epitopes (e.g., on the same or different protein), the fusion proteins are useful in contexts where there is a benefit to bringing different targets into close proximity. Examples of successful implementation of such techniques include emicizumab which acts to bring activated factor IX and factor X together thus enabling the clotting process to continue without needing to replace factor VIII for the treatment of hemophilia.

The fusion proteins of the present invention can also be useful in the field of oncology. For example, depending on the mechanism of action related to an oncology target, cross linking of target cells (e.g., cancer cells) with effector cells (e.g., T cells) may be desirable. Such approaches have proven successful in the context of BiTE® (bispecific T cell engager) antibody constructs. Other examples include trispecifics which can bind two different tumor markers (e.g., via the Ab and/or Fab of the TTR fusion proteins of the present invention) as well as CD3 (e.g., via an anti-CD3 scFv, Ab, or Fab).

The fusion proteins of the present invention can also address the complexities associated with regulatory evaluation/approval of combination treatments. Clinical trials for combination treatments can require more complex clinical trial strategies to evaluate safety and efficacy, especially when none of the individual components have been previously evaluated. The fusion proteins of the present invention address such complexities by combining multiple components into a single construct.

### Methods of Making TTR Heterodimer, Heterotrimer, and Heterotetramer Fusion Proteins

Methods of making the TTR heteromultimer (e.g., heterodimer, heterotrimer, and heterotetramer) fusions of the present invention are discussed in the Examples.

Generally, the TTR heteromultimer (e.g., heterodimer, heterotrimer, and heterotetramer) fusions of the present invention can be generated using recombinant methods. Accordingly, the present invention includes polynucleotides encoding the TTR heteromultimer (e.g., heterodimer, heterotrimer, and heterotetramer) fusions. In another aspect the present invention comprises an expression vector comprising the polynucleotide encoding the TTR heteromultimer (e.g., heterodimer, heterotrimer, and heterotetramer) fusion. In certain embodiments, the expression vectors comprise control sequences (e.g., promoters, enhancers) that are operably linked to a polynucleotide encoding the TTR heteromultimer (e.g., heterodimer, heterotrimer, and heterotetramer) fusion so as support expression in a suitable host cell. In certain embodiments, the expression vector also comprises polynucleotide sequences that allow chromosome-independent replication in the host cell. Exemplary vectors include, but are not limited to, plasmids, cosmids, and YACS. In a particular embodiment, the vector is pTT5.

Generally, mammalian host cells are utilized when generating the TTR heterodimer, heterotrimer, or heterotetramer fusion constructs. Mammalian host cells are also suitable for generating Fab TTR fusion constructs, though non-mammalian cells such as prokaryotic (bacteria) and non-mammalian (e.g., yeast) host cells may also be used.

In yet another aspect, the invention comprises a host cell comprising the expression vector of the invention. Methods of transfecting host cells with the expression vector and culturing the transfected host cells under conditions suitable for expression of the TTR heteromultimer (e.g., heterodimer, heterotrimer, and heterotetramer) fusions are known in the art. The transfection procedure used may depend upon the host to be transformed. Certain methods for introduction of heterologous polynucleotides into mammalian cells are known in the art and include, but are not limited to, dextran-mediated transfection, calcium phosphate precipitation, polybrene mediated transfection, protoplast fusion, electroporation, encapsulation of the polynucleotide(s) in liposomes, and direct microinjection of the DNA into nuclei. Certain mammalian cell lines available as hosts for expression are known in the art and include, but are not limited to, many immortalized cell lines available from the American Type Culture Collection (ATCC), including but not limited to Chinese hamster ovary (CHO; e.g., CHO-K1) cells, E5 cells, baby hamster kidney (BHK) cells, monkey kidney cells (COS), human hepatocellular carcinoma cells (e.g., Hep G2), human embryonic kidney cells 293 (HEK 293), and a number of other cell lines. In certain embodiments, cell lines may be selected through determining which cell lines have high expression levels and produce the TTR heterodimer, heterotrimer, and heterotetramer fusions.

Thus, the present invention also relates to methods of making the TTR heteromultimer (e.g., heterodimer, heterotrimer, and heterotetramer) fusion proteins described herein. For example, the TTR heteromultimer (e.g., heterodimer, heterotrimer, and heterotetramer) fusion proteins may be made by:

a) culturing a recombinant host cell comprising a polynucleotide encoding the TTR heteromultimer (e.g., heterodimer, heterotrimer, and heterotetramer) fusion; and

b) isolating the TTR heteromultimer (e.g., heterodimer, heterotrimer, and heterotetramer) fusion protein from said culture.

### Pharmaceutical Compositions

In some embodiments, the invention provides a pharmaceutical composition comprising a therapeutically effective amount of one or more of the TTR heteromultimer (e.g., heterodimer, heterotrimer, and heterotetramer) fusion proteins of the present invention together with a pharmaceutically effective diluent, carrier, solubilizer, emulsifier, preservative, and/or adjuvant. Pharmaceutical compositions of the invention include, but are not limited to, liquid, frozen, and lyophilized compositions.

Preferably, formulation materials are nontoxic to recipients at the dosages and concentrations employed. In specific embodiments, pharmaceutical compositions comprising a therapeutically effective amount of a TTR heteromultimer (e.g., heterodimer, heterotrimer, and heterotetramer) fusion proteins are provided.

In certain embodiments, the pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine, proline, or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18″ Edition, (A. R. Genrmo, ed.), 1990, Mack Publishing Company.

In certain embodiments, the optimal pharmaceutical composition will be determined by one skilled in the art depending upon, for example, the intended route of administration, delivery format and desired dosage. See, for example, REMINGTON'S PHARMACEUTICAL SCIENCES, supra. In certain embodiments, such compositions may influence the physical state, stability, rate of in vivo release and rate of in vivo clearance of the antigen binding proteins of the invention. In certain embodiments, the primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. In specific embodiments, pharmaceutical compositions comprise Tris buffer of about pH 7.0-8.5, or acetate buffer of about pH 4.0-5.5, and may further include sorbitol or a suitable substitute therefor. In certain embodiments of the invention, TTR heteromultimer (e.g., heterodimer, heterotrimer, and heterotetramer) compositions may be prepared for storage by mixing the selected composition having the desired degree of purity with optional formulation agents (REMINGTON'S PHARMACEUTICAL SCIENCES, supra) in the form of a lyophilized cake or an aqueous solution. Further, in certain embodiments, the TTR heteromultimer (e.g., heterodimer, heterotrimer, and heterotetramer) may be formulated as a lyophilizate using appropriate excipients such as sucrose.

The pharmaceutical compositions of the invention can be selected for parenteral delivery. Alternatively, the compositions may be selected for inhalation or for delivery through the digestive tract, such as orally. Preparation of such pharmaceutically acceptable compositions is within the skill of the art. The formulation components are present preferably in concentrations that are acceptable to the site of administration. In certain embodiments, buffers are used to maintain the composition at physiological pH or at a slightly lower pH, typically within a pH range of from about 5 to about 8.

When parenteral administration is contemplated, the therapeutic compositions for use in this invention may be provided in the form of a pyrogen-free, parenterally acceptable aqueous solution comprising the desired TTR heteromultimer (e.g., heterodimer, heterotrimer, and heterotetramer) in a pharmaceutically acceptable vehicle. A particularly suitable vehicle for parenteral injection is sterile distilled water in which the TTR heteromultimer (e.g., heterodimer, heterotrimer, and heterotetramer) is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that may provide controlled or sustained release of the product which can be delivered via depot injection. In certain embodiments, hyaluronic acid may also be used, having the effect of promoting sustained duration in the circulation. In certain embodiments, implantable drug delivery devices may be used to introduce the desired antigen binding protein.

Pharmaceutical compositions of the invention can be formulated for inhalation. In these embodiments. TTR heteromultimer (e.g., heterodimer, heterotrimer, and heterotetramer) are advantageously formulated as a dry, inhalable powder. In specific embodiments. TTR heteromultimer (e.g., heterodimer, heterotrimer, and heterotetramer) inhalation solutions may also be formulated with a propellant for aerosol delivery. In certain embodiments, solutions may be nebulized. Pulmonary administration and formulation methods therefore are further described in International Patent Application No. PCT/US94/001875, which is incorporated by reference and describes pulmonary delivery of chemically modified proteins.

It is also contemplated that formulations can be administered orally. TTR heteromultimer (e.g., heterodimer, heterotrimer, and heterotetramer) are administered in this fashion can be formulated with or without carriers customarily used in the compounding of solid dosage forms such as tablets and capsules. In certain embodiments, a capsule may be designed to release the active portion of the formulation at the point in the gastrointestinal tract when bioavailability is maximized and pre-systemic degradation is minimized. Additional agents can be included to facilitate absorption of the TTR heteromultimer (e.g., heterodimer, heterotrimer, and heterotetramer). Diluents, flavorings, low melting point waxes, vegetable oils, lubricants, suspending agents, tablet disintegrating agents, and binders may also be employed.

Additional pharmaceutical compositions will be evident to those skilled in the art, including formulations involving TTR heteromultimer (e.g., heterodimer, heterotrimer, and heterotetramer) in sustained- or controlled-delivery formulations. Techniques for formulating a variety of other sustained- or controlled-delivery means, such as liposome carriers, bio-erodible microparticles or porous beads and depot injections, are also known to those skilled in the art. See, for example, International Patent Application No. PCT/US93/00829, which is incorporated by reference and describes controlled release of porous polymeric microparticles for delivery of pharmaceutical compositions. Sustained-release preparations may include semipermeable polymer matrices in the form of shaped articles, e.g., films, or microcapsules. Sustained release matrices may include polyesters, hydrogels, polylactides (as disclosed in U.S. Pat. No. 3,773,919 and European Patent Application Publication No. EP 058481, each of which is incorporated by reference), copolymers of L-glutamic acid and gamma ethyl-L-glutamate (Sidman et al., 1983, Biopolymers 2:547-556), poly (2-hydroxyethyl-methacrylate) (Langer et al., 1981, J. Biomed. Mater. Res. 15:167-277 and Langer, 1982, Chem. Tech. 12:98-105), ethylene vinyl acetate (Langer et al., 1981, supra) or poly-D(−)-3-hydroxybutyric acid (European Patent Application Publication No. EP 133,988). Sustained release compositions may also include liposomes that can be prepared by any of several methods known in the art. See, e.g., Eppstein et al., 1985, Proc. Natl. Acad. Sci. U.S.A. 82:3688-3692; European Patent Application Publication Nos. EP 036,676; EP 088,046 and EP 143,949, incorporated by reference.

Pharmaceutical compositions used for in vivo administration are typically provided as sterile preparations. Sterilization can be accomplished by filtration through sterile filtration membranes. When the composition is lyophilized, sterilization using this method may be conducted either prior to or following lyophilization and reconstitution. Compositions for parenteral administration can be stored in lyophilized form or in a solution. Parenteral compositions generally are placed into a container having a sterile access port, for example, an intravenous solution bag or vial having a stopper pierceable by a hypodermic injection needle.

Aspects of the invention includes self-buffering TTR heteromultimer (e.g., heterodimer, heterotrimer, and heterotetramer) formulations, which can be used as pharmaceutical compositions, as described in international patent application WO 06138181 A2 (PCT/US2006/022599), which is incorporated by reference in its entirety herein.

As discussed above, certain embodiments provide TTR heteromultimer (e.g., heterodimer, heterotrimer, and heterotetramer) compositions, particularly pharmaceutical TTR heteromultimer (e.g., heterodimer, heterotrimer, and heterotetramer) compositions, that comprise, in addition to the heteromultimer, one or more excipients such as those illustratively described in this section and elsewhere herein. Excipients can be used in the invention in this regard for a wide variety of purposes, such as adjusting physical, chemical, or biological properties of formulations, such as adjustment of viscosity, and or processes of the invention to improve effectiveness and or to stabilize such formulations and processes against degradation and spoilage due to, for instance, stresses that occur during manufacturing, shipping, storage, pre-use preparation, administration, and thereafter.

A variety of expositions are available on protein stabilization and formulation materials and methods useful in this regard, such as Arakawa et al., “Solvent interactions in pharmaceutical formulations,” Pharm Res. 8(3): 285-91 (1991); Kendrick et al., “Physical stabilization of proteins in aqueous solution.” in: RATIONAL DESIGN OF STABLE PROTEIN FORMULATIONS: THEORY AND PRACTICE, Carpenter and Manning, eds. Pharmaceutical Biotechnology. 13: 61-84 (2002), and Randolph et al., “Surfactant-protein interactions,” Pharm Biotechnol. 13: 159-75 (2002), each of which is herein incorporated by reference in its entirety, particularly in parts pertinent to excipients and processes of the same for self-buffering protein formulations in accordance with the current invention, especially as to protein pharmaceutical products and processes for veterinary and/or human medical uses.

Salts may be used in accordance with certain embodiments of the invention to, for example, adjust the ionic strength and/or the isotonicity of a formulation and/or to improve the solubility and/or physical stability of a protein or other ingredient of a composition in accordance with the invention.

As is well known, ions can stabilize the native state of proteins by binding to charged residues on the protein's surface and by shielding charged and polar groups in the protein and reducing the strength of their electrostatic interactions, attractive, and repulsive interactions. Ions also can stabilize the denatured state of a protein by binding to, in particular, the denatured peptide linkages (—CONH) of the protein. Furthermore, ionic interaction with charged and polar groups in a protein also can reduce intermolecular electrostatic interactions and, thereby, prevent or reduce protein aggregation and insolubility.

Ionic species differ significantly in their effects on proteins. A number of categorical rankings of ions and their effects on proteins have been developed that can be used in formulating pharmaceutical compositions in accordance with the invention. One example is the Hofmeister series, which ranks ionic and polar non-ionic solutes by their effect on the conformational stability of proteins in solution. Stabilizing solutes are referred to as “kosmotropic.” Destabilizing solutes are referred to as “chaotropic.” Kosmotropes commonly are used at high concentrations (e.g., >1 molar ammonium sulfate) to precipitate proteins from solution (“salting-out”). Chaotropes commonly are used to denture and/or to solubilize proteins (“salting-in”). The relative effectiveness of ions to “salt-in” and “salt-out” defines their position in the Hofmeister series.

Free amino acids can be used in TTR heteromultimer (e.g., heterodimer, heterotrimer, and heterotetramer) formulations in accordance with various embodiments of the invention as bulking agents, stabilizers, and antioxidants, as well as other standard uses. Lysine, proline, serine, and alanine can be used for stabilizing proteins in a formulation. Glycine is useful in lyophilization to ensure correct cake structure and properties. Arginine may be useful to inhibit protein aggregation, in both liquid and lyophilized formulations. Methionine is useful as an antioxidant.

Polyols include sugars, e.g., mannitol, sucrose, and sorbitol and polyhydric alcohols such as, for instance, glycerol and propylene glycol, and, for purposes of discussion herein, polyethylene glycol (PEG) and related substances. Polyols are kosmotropic. They are useful stabilizing agents in both liquid and lyophilized formulations to protect proteins from physical and chemical degradation processes. Polyols also are useful for adjusting the tonicity of formulations.

Among polyols useful in select embodiments of the invention is mannitol, commonly used to ensure structural stability of the cake in lyophilized formulations. It ensures structural stability to the cake. It is generally used with a lyoprotectant, e.g., sucrose. Sorbitol and sucrose are among preferred agents for adjusting tonicity and as stabilizers to protect against freeze-thaw stresses during transport or the preparation of bulks during the manufacturing process. Reducing sugars (which contain free aldehyde or ketone groups), such as glucose and lactose, can glycate surface lysine and arginine residues. Therefore, they generally are not among preferred polyols for use in accordance with the invention. In addition, sugars that form such reactive species, such as sucrose, which is hydrolyzed to fructose and glucose under acidic conditions, and consequently engenders glycation, also is not among preferred polyols of the invention in this regard. PEG is useful to stabilize proteins and as a cryoprotectant and can be used in the invention in this regard.

Embodiments of the TTR heteromultimer (e.g., heterodimer, heterotrimer, and heterotetramer) formulations further comprise surfactants. Protein molecules may be susceptible to adsorption on surfaces and to denaturation and consequent aggregation at air-liquid, solid-liquid, and liquid-liquid interfaces. These effects generally scale inversely with protein concentration. These deleterious interactions generally scale inversely with protein concentration and typically are exacerbated by physical agitation, such as that generated during the shipping and handling of a product.

Surfactants routinely are used to prevent, minimize, or reduce surface adsorption. Useful surfactants in the invention in this regard include polysorbate 20, polysorbate 80, other fatty acid esters of sorbitan polyethoxylates, and poloxamer 188.

Surfactants also are commonly used to control protein conformational stability. The use of surfactants in this regard is protein-specific since, any given surfactant typically will stabilize some proteins and destabilize others.

Polysorbates are susceptible to oxidative degradation and often, as supplied, contain sufficient quantities of peroxides to cause oxidation of protein residue side-chains, especially methionine. Consequently, polysorbates should be used carefully, and when used, should be employed at their lowest effective concentration. In this regard, polysorbates exemplify the general rule that excipients should be used in their lowest effective concentrations.

Embodiments of TTR heteromultimer (e.g., heterodimer, heterotrimer, and heterotetramer) formulations further comprise one or more antioxidants. To some extent deleterious oxidation of proteins can be prevented in pharmaceutical formulations by maintaining proper levels of ambient oxygen and temperature and by avoiding exposure to light. Antioxidant excipients can be used as well to prevent oxidative degradation of proteins. Among useful antioxidants in this regard are reducing agents, oxygen/free-radical scavengers, and chelating agents. Antioxidants for use in therapeutic protein formulations in accordance with the invention preferably arm water-soluble and maintain their activity throughout the shelf life of a product. EDTA is a preferred antioxidant in accordance with the invention in this regard.

Antioxidants can damage proteins. For instance, reducing agents, such as glutathione in particular, can disrupt intramolecular disulfide linkages. Thus, antioxidants for use in the invention are selected to, among other things, eliminate or sufficiently reduce the possibility of themselves damaging proteins in the formulation.

Formulations in accordance with the invention may include metal ions that are protein co-factors and that are necessary to form protein coordination complexes, such as zinc necessary to form certain insulin suspensions. Metal ions also can inhibit some processes that degrade proteins. However, metal ions also catalyze physical and chemical processes that degrade proteins.

Magnesium ions (10-120 mM) can be used to inhibit isomerization of aspartic acid to isoaspartic acid. Ca⁺² ions (up to 100 mM) can increase the stability of human deoxyribonuclease. Mg⁺². Mn⁺², and Zn⁺², however, can destabilize rhDNase. Similarly, Ca⁺² and Sr⁺² can stabilize Factor VIII, it can be destabilized by Mg⁺², Mn⁺² and Zn⁺², Cu⁺² and Fe⁺², and its aggregation can be increased by Al⁺³ ions.

Embodiments of the TTR heteromultimer (e.g., heterodimer, heterotrimer, and heterotetramer) formulations further comprise one or more preservatives. Preservatives are necessary when developing multi-dose parenteral formulations that involve more than one extraction from the same container. Their primary function is to inhibit microbial growth and ensure product sterility throughout the shelf-life or term of use of the drug product. Commonly used preservatives include benzyl alcohol, phenol and m-cresol. Although preservatives have a long history of use with small-molecule parenterals, the development of protein formulations that includes preservatives can be challenging. Preservatives almost always have a destabilizing effect (aggregation) on proteins, and this has become a major factor in limiting their use in multi-dose protein formulations. To date, most protein drugs have been formulated for single-use only. However, when multi-dose formulations are possible, they have the added advantage of enabling patient convenience, and increased marketability. A good example is that of human growth hormone (hGH) where the development of preserved formulations has led to commercialization of more convenient, multi-use injection pen presentations. At least four such pen devices containing preserved formulations of hGH are currently available on the market. Norditropin (liquid, Novo Nordisk), Nutropin AQ (liquid, Genentech) & Genotropin (lyophilized—dual chamber cartridge, Pharmacia & Upjohn) contain phenol while Somatrope (Eli Lilly) is formulated with m-cresol.

Several aspects need to be considered during the formulation and development of preserved dosage forms. The effective preservative concentration in the drug product must be optimized. This requires testing a given preservative in the dosage form with concentration ranges that confer anti-microbial effectiveness without compromising protein stability.

As might be expected, development of liquid formulations containing preservatives are more challenging than lyophilized formulations. Freeze-dried products can be lyophilized without the preservative and reconstituted with a preservative containing diluent at the time of use. This shortens the time for which a preservative is in contact with the protein, significantly minimizing the associated stability risks. With liquid formulations, preservative effectiveness and stability should be maintained over the entire product shelf-life (e.g., about 18 to 24 months). An important point to note is that preservative effectiveness should be demonstrated in the final formulation containing the active drug and all excipient components.

TTR heteromultimer (e.g., heterodimer, heterotrimer, and heterotetramer) formulations generally will be designed for specific routes and methods of administration, for specific administration dosages and frequencies of administration, for specific treatments of specific diseases, with ranges of bioavailability and persistence, among other things. Formulations thus may be designed in accordance with the invention for delivery by any suitable route, including but not limited to orally, aurally, ophthalmically, rectally, and vaginally, and by parenteral routes, including intravenous and intraarterial injection, intramuscular injection, and subcutaneous injection.

Once the pharmaceutical composition has been formulated, it may be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration. The invention also provides kits for producing a single-dose administration unit. The kits of the invention may each contain both a first container having a dried protein and a second container having an aqueous formulation. In certain embodiments of this invention, kits containing single and multi-chambered pre-filled syringes (e.g., liquid syringes and lyosyringes) are provided.

The therapeutically effective amount of a TTR heteromultimer-containing (e.g., heterodimer, heterotrimer, and heterotetramer) pharmaceutical composition to be employed will depend, for example, upon the therapeutic context and objectives. One skilled in the art will appreciate that the appropriate dosage levels for treatment will vary depending, in part, upon the molecule delivered, the indication for which the TTR heteromultimer (e.g., heterodimer, heterotrimer, and heterotetramer) is being used, the route of administration, and the size (body weight, body surface or organ size) and/or condition (the age and general health) of the patient. In certain embodiments, the clinician may titer the dosage and modify the route of administration to obtain the optimal therapeutic effect. A typical dosage may range from about 0.1 μg/kg to up to about 30 mg/kg or more, depending on the factors mentioned above. In specific embodiments, the dosage may range from 1.0 μg/kg up to about 20 mg/kg, optionally from 10 μg/kg up to about 10 mg/kg or from 100 μg/kg up to about 5 mg/kg.

A therapeutic effective amount of a TTR heteromultimer (e.g., heterodimer, heterotrimer, and heterotetramer) preferably results in a decrease in severity of disease symptoms, in an increase in frequency or duration of disease symptom-free periods, or in a prevention of impairment or disability due to the disease affliction.

Pharmaceutical compositions may be administered using a medical device. Examples of medical devices for administering pharmaceutical compositions are described in U.S. Pat. Nos. 4,475,196; 4,439,196; 4,447,224; 4,447,233; 4,486,194; 4,487,603; 4,596,556; 4,790,824; 4,941,880; 5,064,413; 5,312,335; 5,312,335; 5,383,851; and 5,399,163, all incorporated by reference herein.

### Therapeutic Uses of TTR Heterodimer, Heterotrimer, and Heterotetramer Fusion Proteins

As illustrated in the Examples, it has been discovered that the multispecific TTR fusion proteins of the present invention are capable of binding two or more epitopes on one or more proteins. Such multispecific TTR fusions are particularly useful in that they can engage multiple biological pathways allowing for a more effective treatment of disease states (e.g., cancer) compared to traditional modes of treatment.

The multispecific TTR fusion proteins of the present invention have benefits over many known bispecific/multispecific approaches. For example, the present invention provides bivalent bispecific presentation of antigen binding domains which reduces or eliminates avidity loss compared to, e.g., hetero-IgG constructs. Additional benefits over hetero-IgG constructs include that the multispecific TTR fusion proteins of the present invention can be generated without the need for Fc charge pair mutations (CPMs) which are needed to drive heterodimerization of heavy chains in hetero-IgG constructs, as well as the reduction or elimination of undesirable side-products such as half-antibody and light chain mismatches (present in hetero-IgG and IgG-Fab constructs). Indeed, the TTR fusion proteins of the present invention reduce much (and in some cases, all) of the Ab or Fab engineering needed in other constructs.

Compared to IgG-Fab and IgG-scFv constructs, the antigen binding domains of the TTR fusion proteins of the present invention are optimally oriented such that the N-terminus antigen binding regions are exposed and steric induced affinity loss is reduced or eliminated.

Another benefit of the TTR fusion proteins of the present invention stems from the use of native IgG formats which helps reduce the affinity loss and increase in aggregation propensity observed when converting mAbs into scFv constructs. Gil and Schrum, Advances in Bioscience and Biotechnology, 4:73-84 (2013).

In addition, because the TTR fusion proteins of the present invention allow for the efficient incorporation of a variety of antigen-binding domains, rapid scanning of bispecific (or multispecific) combinations is enabled.

The TTR heteromultimer (e.g., heterodimer, heterotrimer, and heterotetramer) fusion proteins also demonstrate improved antigen clustering compared to the individual antibody(ies) and/or Fab(s). When antibodies (e.g., IgG antibodies) bind antigens on target cells (e.g., tumor cells), the resulting clustered Fc domains engage with FcγR's found on immune effector cells such as NK cells and macrophages. This clustering aids in signaling through FcγR resulting in the initiation of cell-mediated effector functions such as antibody-dependent cellular cytotoxicity (ADCC) and antibody-dependent cellular phagocytosis (ADCP). Thus, the TTR heteromultimer (e.g., heterodimer, heterotrimer, and heterotetramer) fusion proteins are particularly useful in targeting ligands where high antibody or Fab affinity/avidity leads to an enhanced biological effect. Enhancement of cell-mediated effector functions by the TTR heteromultimer (e.g., heterodimer, heterotrimer, and heterotetramer) constructs of the present invention results in an increased ability to kill cells which is useful, e.g., the treatment of cancer.

Accordingly, the present invention also relates to methods of treating cancer using the heterodimer fusion proteins and heterotetramer fusion proteins described herein.

In other embodiments, the present invention relates to a use of the heterodimer fusion proteins and heterotetramer fusion proteins described herein in the treatment of cancer.

In yet other embodiments, the present invention relates to heterodimer fusion proteins and heterotetramer fusion proteins described herein for use in the treatment of cancer.

### EXAMPLES

The following examples are provided for the purpose of illustrating specific embodiments or features of the present invention and are not intended to limit its scope.

### Example 1: General Techniques

Example 1 describes general techniques that were employed to make and characterize the TTR negative and positive constructs discussed in the rest of the Examples.

The following techniques were used to generate TTR negative and positive constructs containing TTR variants with one TTR dimer/dimer interface mutation (“C10A/K15A/XX”) per TTR subunit

Cloning of TTR Negative and Positive Variants in E. coli

Amgen Large Molecule Registry Construct C37979 (pAMG21: huTTR (opt-C10A, K15A)) was used as a template for all the TTR negative and positive variants (containing C10A/K15A/XX). TTR negative and positive variants were generated using standard molecular biology techniques including polymerase chain reaction (PCR), site-directed PCR mutagenesis, restriction endonuclease digestion and enzymatic ligation into bacterial expression plasmids. TTR negative and positive variants containing a MKH6GG at the TTR N-terminus were also generated.

The techniques were generally performed according to methods that can be reference in Molecular Cloning: A Laboratory Manuel, 3rd ed., Sambrook et al., 2001, Cold Spring Harbor Laboratory Press, cold Spring Harbor, N.Y.

Expression of TTR Negative and Positive Variants in E. coli

BL21 cells containing pAMG21 vector encoding the TTR negative and positive variants were grown overnight at 30-37° C. in a 50 ml volume of Terrific Broth (Teknova T7060) with 20 μg/ml Kanamycin in a 250 ml baffled shake flask. The next day, 35 ml overnight culture was added to 1 L Terrific Broth with 20 μg/ml Kanamycin and 50 μl Sigma Y-30 antifoam and incubated at 33° C. until the OD at 600 nm had reached 0.4. 1 ml of Sigma K-3255 N-(beta-Ketocaproyl)-DL-homoserine lactone autoinducer (stock solution dissolved in ethanol) was added to the culture which was left to express for four hours at 30 to 33° C.

Purification of the TTR Negative and Positive Variants from E. coli

Frozen E. coli cell paste was homogenized in a 1:10, weight to volume, 50 mM Na-phosphate, 300 mM NaCl, pH 8.0 solution using an Omni TH (Omni International, Kennesaw, Ga., USA) handheld homogenizer. The resultant suspension was then twice processed through an M-110S Microfluidizer (Microfluidics Corporation, Irvine, Calif., USA) at 13,800 PSI. The lysate was then centrifuged at 22,000 RCF for 1 hour at 4° C. The soluble fraction was filtered through a 0.45 μm cellulose acetate filter (Corning Life Sciences, Tewksbury, Mass., USA) and retained as starting material for FPLC purification; the insoluble fraction was disposed of as waste.

A 5 mL Ni-NTA SuperFlow column (Qiagen, Hilden, Germany) connected to an ÄKTApurifier (GE Healthcare Bio-Sciences, Marlborough, Mass., USA) FPLC was equilibrated with 5 column volumes (CV) of 50 mM Na-phosphate, 300 mM NaCl, 10 mM imidazole, pH 8.0 prior to sample application. The filtered soluble lysate was injected onto the column, washed with 15 CV of 50 mM Na-phosphate, 300 mM NaCl, 10 mM imidazole, pH 8.0, and eluted stepwise with 10 CV of 50 mM Na-phosphate, 300 mM NaCl, 250 mM imidazole, pH 8.0.

The purification pool was concentrated using a VivaSpin 10 kDA MWCO (Sartorius AG, Gottingen, Germany) centrifugal filter, centrifuged at 3,000 RCF until the desired volume was reached.

The concentrated sample was dialyzed against 10 mM tris-HCl, pH 8.0. 150 mM NaCl using Slide-a-lyzer 10 kDa MWCO (Thermo Fisher Scientific, Waltham, Mass., USA) dialysis cartridge until the starting buffer was below 1%, by calculation.

PC Analytics of the TTR Negative and Positive Variants (E. coli)

Protein quantitation was performed by measuring UV absorbance at 280 nm using a Nanodrop 2000c (Thermo Fisher Scientific).

Non-reducing SDS-PAGE analysis was performed with and without sample heating. In both cases, the sample was treated with SDS-PAGE Sample Buffer and run on a 4-20% Tris-Gly SDS-PAGE (Thermo Fisher Scientific), per manufacturer protocol. In the heated experiment, the sample and Sample Buffer solution was heated at 85° C. for 5 minutes, then loaded onto the gel; the unheated sample was loaded directly onto the gel after Sample Buffer addition. The gel was stained using SimplyBlue SafeStain (Thermo Fisher Scientific) per manufacturer microwave protocol.

HPLC SEC analysis was performed on an SEC-3000, 7.8×300 mm column (Phenomenex, Torrance, Calif., USA) connected to an Agilent 1290 Infinity HPLC system (Agilent Technologies, Santa Clara, Calif., USA) running an isocratic 50 mM NaH2PO4, 250 mM NaCl, pH 6.9 mobile phase at 1 mL/min and observing UV absorbance at 280 nm.

### TTR Negative and Positive Variants Dimerization

Purified TTR samples were normalized to the lowest common molar concentration of the experimental cohort by dilution with 10 mM tris-HCl, pH 8.0, 150 mM NaCl. Samples were combined in equal volumes and incubated overnight at 4° C.

Part of the mixed samples were processed by caspase cleavage as follows. Purified protein sample concentrations were adjusted to 2.5 mg/mL by dilution using 10 mM tris-HCl, pH 8.0, 150 mM NaCl. Prepared a 5× Digestion Buffer consisting of 250 mM NaCl, 15 mM 2-mercaptoethanol, pH 8.0 and brought to 25° C. in a water bath, as well as a 1× Digestion Buffer by diluting the 5× buffer with water. Diluted a stock aliquot of Caspase-3 (Amgen Inc., Thousand Oaks, Calif., USA) to 0.1 mg/mL using 1× Digestion Buffer. Combined 4 parts protein, 4 parts diluted Caspase-3, 8 parts 5× Digestion Buffer and 20 parts water, and incubated at 25° C. in a water bath for 2 hours. Removed digest solution and added 20 parts SDS-PAGE Sample Buffer (the same reagent specified previously for SDS-PAGE analysis). Ran cleavage reaction on SDS-PAGE using the same protocol specified previously.

The resulting molecule mixtures, caspase process and not, were analyzed by non-reducing, unheated SDS-PAGE and HPLC SEC.

Cloning of the (1) [Ab “A”]=[negative TTR]₂: [positive TTR]₂=[Ab “B”] (2× Ab-TTR); (2) [[Ab “A”]-[negative TTR]]₂: [[positive TTR]-[Ab “B”]]₂ (4× Ab-TTR); and (3) [[Fab “A”]-[negative TTR]]₂: [[positive TTR]-[Fab “B”]]₂ (4× Fab-TTR) Molecules (No Linker)

TTR was fused to several engineered variants of hybridoma derived anti-CB1, anti-GITR and anti-TR2 antibody heavy chain (HC) using standard molecular biology techniques including polymerase chain reaction (PCR), site-directed PCR mutagenesis, restriction endonuclease digestion and enzymatic ligation into mammalian expression plasmids. Also generated were His-tagged Fab-TTR molecules. These cloned TTR fused variant heavy chain and Fab DNAs in combination with their respective cloned anti-CB1, anti-GITR and anti-TR2 antibody light chain (LC) DNAs were used to transfect mammalian cell for the expression of the 2× Ab-TTR, 4× Ab-TTR, and 4× Fab-TTR. The techniques were generally performed according to methods that can be reference in Molecular Cloning: A Laboratory Manuel, 3^{rd} ed., Sambrook et al., 2001, Cold Spring Harbor Laboratory Press, cold Spring Harbor, N.Y.

TTR antibody and Fab fusion sequences were generated by GENEART® Seamless Cloning (GSC) or Golden Gate Assembly (GGA). The DNA fragments that were combined were produced by splicing overlap extension PCR (SOE-PCR) or were ordered synthetically from an external vendor. SOE-PCR products utilized in GSC cloning were created using flanking primers paired with mutagenic palindromic primers that created two PCR products that shared a 15 bp overlap region surrounding the codon for the desired amino acid change site. SOE-PCR products used in GGA were designed to include directional unique 4 base pair overhangs that were generated by BsmBI digestion.

Briefly, GGA relied upon Type II restriction enzymes and T4 DNA ligase to cut and seamlessly ligate together multiple DNA fragments. (Engler et al., PLOS One, Vol. 3(11): e3647, 2008). In this example, the multiple DNA fragments consisted of (i) a synthetic nucleic acid sequence (GeneByte, Gen9, Cambridge, Mass.) encoding a Kozak consensus sequence, a signal peptide sequence, a full antibody gene, a linker and a TTR sequence and (ii) the expression vector backbone. The GGA reactions were composed of 50 ng of GeneByte, 20 ng of the expression vector, 1 μl 10× Fast Digest Reaction Buffer+0.5 mM ATP (Thermo Fisher, Waltham, Mass.), 0.5 μl FastDigest Esp3I (Thermo Fisher, Waltham, Mass.), 1 μl T4 DNA Ligase (5U/μl, Thermo Fisher, Waltham, Mass.) and water to 10 μl. The reactions were performed over 15 cycles consisting of a 2-minute digestion step at 37° C. and a 3-minute ligation step at 16° C. The 15 cycles were followed by a final 5 minute 37° C. digestion step and a 5-minute enzyme inactivation step at 80° C.

Expression of the [Ab “A”]=[negative TTR]₂: [positive TTR]₂=[Ab “B”] Molecules (No Linker)

HEK 293-6E cells were maintained in FreeStyle F17 Medium (Thermo Fisher Scientific) supplemented with 0.1% (w/v) Poloxamer 188 (Sigma-Aldrich), 6 mM L-Glutamine (Thermo Fisher Scientific), 25 μg/ml G418 (Thermo Fisher Scientific) at 36° C. in shaker flask in an incubator with 5% CO₂, 80%-90% humidity and 120 rpm agitation on a shaker of 25 mm shaking diameter. The 293-6E cells were seeded 2 days prior to transfection at 0.4×10⁶ cells/ml. On the day of transfection, the cells were at the exponential growth phase (˜1.5×10⁶ cells/ml, >95% viability). Transient transfections were performed at 20% gene dose by adding the mixture of 0.5 mg/L DNA (0.1 mg/L gene of interest construct DNA+0.4 mg/L vector DNA) and 2 mg/L PEI Max (Polyethylenimine Max, Polysciences, Cat #24765-2) to the cell culture. Proprietary feeds {Yeastolate (0.5% w/v) and glucose (3 g/L)} were added 4 hours post transfection. Productions were harvested 6 days post transfection by centrifuging cells at 4000 rpm (3485×g) for 40 minutes. The supernatant was filtered with 0.45 μM PES (polyethersulfone) filter.

Purification of the [Ab “A”]=[negative TTR]₂: [positive TTR]₂=[Ab “B”] Molecules (No Linker)

A rProtein A Fast Flow column (GE Healthcare Bio-Sciences, Marlborough, Mass., USA) connected to an ÄKTApurifier (GE Healthcare Bio-Sciences) FPLC was equilibrated with Dulbecco's PBS (DPBS) prior to sample application. The filtered cell culture media was injected onto the column, washed with 5 column volumes (CV) of DPBS, and eluted stepwise with 8 CV of 50 mM HOAc, pH 3.2. The eluate was titrated to pH 5.0 using 1 M tris, then filtered through a 0.45 μm cellulose acetate vacuum filter (Corning Inc., Corning, N.Y., USA). The titrated and filtered rProtein A pool was divided into two separate pools for additional purification.

One half of the rProtein A pool was diluted 1:5, by volume, with 20 mM MES, pH 5.0, then injected onto an SP Sepharose High Performance column (GE Healthcare Bio-Sciences) connected to an ÄKTApurifier (GE Healthcare Bio-Sciences) FPLC, previously equilibrated with 20 mM NaOAc, pH 5.0. The column was washed with 5 CV of 20 mM NaOAc, pH 5.0, and eluted with a 20 CV gradient from 20 mM NaOAc, pH 5.0 to 20 mM NaOAc, 500 mM NaCl, pH 5.0.

The SP Sepharose fractions were analyzed on a Caliper LabChip GXII microcapillary electrophoresis system using the Protein Express Assay LabChip (Perkin Elmer, Waltham, Mass., USA) per manufacturer protocol. Fractions were selected for enrichment of the band at the approximate molecular weight of monomeric Ab-TTR versus non-conforming MW species, then pooled.

The SP Sepharose pool was dialyzed against 10 mM MES, 150 mM NaCl, pH 6.5 using Slide-a-lyzer 10 kDa MWCO (Thermo Fisher Scientific, Waltham, Mass., USA) dialysis cartridge until the starting buffer was below 1%, by calculation.

The other half of the rProtein A pool was injected onto a Sephadex G-25 (GE Healthcare Bio-Sciences) column connected to an ÄKTApurifier (GE Healthcare Bio-Sciences) FPLC, previously equilibrated with 20 mM MES, pH 6.5. The column was eluted isocratically in 10 mM MES, 150 mM NaCl, pH 6.5.

PC Analytics of the [Ab “A”]=[negative TTR]₂: [positive TTR]₂=[Ab “B”] Molecules (No Linker)

Protein quantitation was performed by measuring UV absorbance at 280 nm using a Nanodrop 2000c (Thermo Fisher Scientific).

Non-reducing SDS-PAGE analysis was performed by treating the sample with SDS-PAGE Sample Buffer (Thermo Fisher Scientific) containing 100 mM iodoacetamide (Sigma-Aldrich, St. Louis, Mo., USA), then loaded directly onto a 10% Tris-Gly gel and run per manufacturer protocol. Reducing SDS-PAGE analysis was performed by treating the sample with SDS-PAGE Sample Buffer and Sample Reducing Agent (Thermo Fisher Scientific). The sample was incubated at 85° C. for 5 minutes, then loaded on a 10% Tris-Gly gel and run per manufacturer protocol. The gels were stained using SimplyBlue SafeStain (Thermo Fisher Scientific) per manufacturer microwave protocol.

HPLC SEC analysis was performed on a Zenix-C SEC-300, 7.8×300 mm column (Sepax Technologies Inc., Newark, Del., USA) connected to an Agilent 1290 Infinity HPLC system (Agilent Technologies, Santa Clara, Calif., USA) running an isocratic 50 mM NaH2PO4, 250 mM NaCl, pH 6.9 mobile phase at 1 mL/min and observing UV absorbance at 280 nm.

DASAnalytics of the [Ab “A”]=[negative TTR]₂: [positive TTR]₂=[Ab “B”] Molecules (No Linker)

Denaturing LC-MS: all LC-MS data was acquired on an Agilent 6230 TOF LC/MS system with a 1290 Infinity LC system. Chromatographic separation was achieved using a Zorbax SB300-C8 3.5 μm 2.1×50 mm column operated at a temperature of 75° C. The solvents used were as follows: mobile phase A was water containing 0.1% v/v TFA. Mobile phase B was 90% n-propanol containing 0.1% v/v TFA. Initial gradients conditions were 20% mobile phase B from 0.0 to 1.0 minutes; 1.0 to 9.0 minutes, 20-70% mobile phase B; 9.0-10.0 minutes, 70-100% mobile phase B, where it remains at 100% for 1 further minute. The flow rate was 0.2 mL/min. Approximately 5 μg of IgG1-biotin conjugate was loaded on to the LC-MS system for each analyses. Data was acquired over the m/z range 1000-7000. The source fragmentor, skimmer and octapole 1 RF values were: 460 V, 95 V and 800 V (peak-to-peak) respectively. The ESI capillary voltage was 5.9 kV. Gas temperature was 340° C. Drying gas was 13 L/min. Nebulizer was 25 psig. Oa-ToF calibration was performed using the Agilent Tune Mix using the automated calibration procedure implemented through MassHunter Data Acquisition version B.06.01, Build 6.01.6157.

Expression of the [655-341 Ab]-[[LX]-[negative TTR]]₂: [[positive TTR] [LX]]₂=[655-341 Ab] Molecules

Expression was conducted as described for the [Ab “A”]=[negative TTR]₂: [positive TTR]₂=[Ab “B”] Molecules.

Purification of the [655-341 Ab]=[[LX]—[negative TTR]]₂:[[positive TTR]-[LX]]₂=[655-341 Ab] Molecules

The filtered cell culture media was injected onto a rProtein A Fast Flow HiTrap columne (GE Healthcare Bio-Sciences) and Desalting HiTrap column (GE Healthcare Bio-Sciences) in-line tandem purification system, equilibrated with DPBS and 10 mM MES 150 mM NaCl, pH 6.5, respectively, connected to an ÄKTApurifier (GE Healthcare Bio-Sciences). The rProtein A column was washed with DPBS, and eluted stepwise with 100 mM HOAc, pH 3.6. The rProtein A eluate was buffer exchanged on the Desalting HiTrap column with 10 mM MES, 150 mM NaCl, pH 6.5.

DAS Analytics of the [655-341 Ab]=[[LX] [negative TTR]]₂: [[positive TTR]-[LX]]₂=[655-341 Ab] Molecules

Denaturing LC-MS was conducted as described for the [Ab “A”]=[negative TTR]₂: [positive TTR]₂=[Ab “B”] Molecules.

Expression of the (1) [[Fab “A”]-[negative TTR]]₂: [[positive TTR]-[Fab “B”]]₂. (2) [Ab “A”]=[negative TTR]₂: [positive TTR]₂=[Ab “B”], and (3) [Ab “A”]=[negative TTR]₂: [[positive TTR]-[Fab “B”]]₂ Molecules (Co-Expression)

CHO-K1 growth media consists of 50% CS9 Media (non-select, Amgen proprietary)+50% ExCell302 (SAFC Biosciences #14324C)+2 mM L-glutamine (Gibco #25030-081). Selection Media consists of growth media+10 ug/ml puromycin (Gibco #A11138-03)+500 μg/ml hygromycin (Invitrogen #10687-010). Production media consists of CHO-K1 6DCD (ATO Media Lab, Amgen proprietary).

Transfection Reagents consist of Lipofectamine LTX (Gibco #15338-100 (p/n 94756)) and Opti-MEM I Reduced Serum Media (Gibco #31985-070). Growth Conditions were suspension growth at 36° C.+5% CO₂ in a humidified incubator shaking at 120 RPM using vented shake flasks. Transfection procedure was as follows. The day before transfection, host culture was split to between 7-10 e⁵ VCD/ml. DNA/Lipofectamine LTX complex was prepared as follows. 4 μg non-linearized DNA was diluted in 0.5 ml Opti-MEM media in a 24DWB (2.0 μg GOI (gene of interest) and 2.0 μg of PB200 (hyperactive transposase)). For four chain transfections 0.5 ug of each chain and 2.0 μg of PB200 (hyperactive transposase) was used for a total of 4.0 μg/transfection. For three chain transfections 0.66 μg of each chain and 2.0 μg of PB200 (hyperactive transposase) was used for a total of 4.0 μg/transfection. 10 μl Lipofectamine LTX was diluted in 0.5 ml Opti-MEM media in a 15 ml polypropylene tube, and sit for 5 minutes. Diluted DNA was then combined with Lipofectamine LTX and mixed thoroughly by pipetting. The mixture was incubated at room temperature for 15-20 minutes, mixing occasionally. 2e⁶ viable cells/transfection were then transferred to a 15-50 ml polypropylene tube, spin @ 1200 rpm for 5 minutes, aspirate media. The cells were then washed with 1×PBS via complete resuspension and spun d; 1200 rpm for 5 minutes. The 1×PBS was then aspirated and the cells were resuspend in 1 ml of Opti-MEM (per transfection). 1 ml cells was then added to each well followed by DNA/LTX complex drop-wise to each well. Cells were incubated for 5-6 hours shaking at 235 rpm, 36° C.+5% CO². 2.0 ml non-select growth media (CHO-K1 Media) was then added to the cells. Selection at 72 hours post transfection was done by placing the cells into 4 ml selection media through complete resuspension. The day 6 expansion scale-up was carried out by adding 1.6 ml from the DWB culture directly to 12 ml in a 50 ml vented spin tube. Day 10 production was done by inoculating a 40 ml batch production through resuspension of ˜13 ml N−1 culture in production media. Day 17 harvest was carried out by centrifugal separation of the cells followed by sterile filtration of the conditioned media.

Purification of the [[Fab “A”]-[negative TTR]]₂: [[positive TTR]-[Fab “B”]]₂ Molecules (Co-Expression)

Fab-TTR fusion proteins included a C-terminus 6× his-tag and were captured from the CM through IMAC affinity chromatography (1 ml HisTrap Excel, GE Healthcare, 17-3712-05) at a flowrate of 2 ml/minute. The IMAC column was then washed with 5 CV of 20 mM sodium phosphate, 250 mM sodium chloride, pH 7.4 at a flowrate of 4 ml/minute by stepwise protein elution with 20 mM sodium phosphate, 250 mM sodium chloride, 0.5 M imidazole, pH 7.4 at 2 ml/minute. The IMAC column was stripped with 6 M guanidine-HCl, 50 mM Tris, pH 8 and equilibrated with 20 mM sodium phosphate, 250 mM sodium chloride, pH 7.4 prior to the next sample loading.

All purified proteins were finally buffer exchanged into 10 mM MES, 150 mM sodium chloride, pH 6, by passing over a 5 ml HiTrap Desalting column (GE Healthcare; 17-1408-01) at a flowrate of 2 ml/minute. All preparative chromatography operations were performed using ÄKTA Purifiers (GE Healthcare). A combination of analytical methods was then utilized to characterize the amount and quality of the proteins produced, including A280 protein quantitation, size-exclusion chromatography (SEC), microcapillary electrophoresis (MCE), and SDS-PAGE.

Purification of the [Ab “A”]=[negative TTR]₂: [positive TTR]₂: =[Ab “B”] and [Ab “A”]=[negative TTR]₂: [[positive TTR]-[Fab “B”]]₂ Molecules (Co-Expression)

Antibody-TTR fusion proteins were captured from the CM through Protein A affinity chromatography (1 ml MabSelect SuRe HiTrap, GE Healthcare, Bio-Sciences, Marlborough, Mass., USA; 11-0034-93) at a flowrate of 2 ml/minute. The Protein A column was then washed with 5 CV of 25 mM Tris, 100 mM sodium chloride, pH 7.4 at a flowrate of 4 ml/minute by stepwise protein elution with 100 mM acetic acid, pH 3.6 at 2 ml/minute. The column was stripped with 6 M guanidine-HCl, 50 mM Tris, pH 8 and equilibrated with 25 mM Tris, 100 mM sodium chloride, pH 7.4 prior to the next sample loading.

All purified proteins were finally buffer exchanged into 10 mM MES, 150 mM sodium chloride, pH 6, by passing over a 5 ml HiTrap Desalting column (GE Healthcare; 17-1408-01) at a flowrate of 2 ml/minute. All preparative chromatography operations were performed using ÄKTA Purifiers (GE Healthcare).

PC Analytics of the (1) [[Fab “A”] [negative TTR]]₂: [[positive TTR]-[Fab “B”]]₂. (2) [Ab “A”]=[negative TTR]₂: [positive TTR]₂=[Ab “B”], and (3) [Ab “A”]=[negative TTR]₂: [[positive TTR]-[Fab “B”]]₂ Molecules (Co-Expression)

A280 Quantitation—Protein quantitation was performed by measuring UV absorbance at 280 nm using a Multiskan Go (Thermo Fisher Scientific, Waltham, Mass., USA).

SEC—TTR-fusion protein samples were applied to an ACQUITY UPLC BEH 200 Å, 1.7 μm, 4.6×300 mm SEC column (Waters. Milford, Mass., USA; 186005226) at a flow rate of 0.4 ml/minute in a mobile phase of 100 mM sodium phosphate, 50 mM sodium chloride, 7.5% ethanol, pH 6.9 and observing UV absorbance at 280 nm. The analytical SEC was performed using a 1290 Infinity HPLC (Agilent Technologies, Santa Clara, Calif. USA). Due to the large MW of these TTR-fusion molecules (249-347 kDa) and product-related impurities, MW benchmark molecules were utilized to gauge the approximate SEC retention times of the expected MW for the specific fusion molecule. These benchmark molecules were Amgen, Inc. produced molecules, and included 2 different antibodies (each 145 kDa; protein lots BR4214-1 and PL41591), antibody-TTR heterotetramer (635 kDa; protein lot PL38002), antibody-TTR heterodimer (265 kDa; protein lot PL46796), and Fab-TTR heterotetramer (248 kDa; protein lot PL38000).

MCE—Characterization of the TTR-fusion protein samples by microcapillary electrophoresis was performed using the LabChip GXII (Caliper LifeSciences, Mountainview, Calif., USA). Samples were prepared reduced and non-reduced per the manufacturer's guidelines. The microfluidics chip technology automatically stains, destains, electrophoretically separates, and analyzes protein samples.

SDS-PAGE-TTR-fusion protein samples were run on a variety of Tris-Glycine, one-dimensional gels, including 8%, 10%, and 4-20% (Invitrogen, Carlsbad, Calif., USA; Wedge Well: XP00080, XP00100, XP04200, respectively). Samples were prepared non-reduced, either unheated or heated at 85′ C for 10 minutes. Gels were stained using SimpyBlue SafeStain (Invitrogen; LC6060) and compared to a MW reference standard for identification of desired product bands.

DAS Analytics of the (1) [[Fab “A”]-[negative TTR]]₂: [[positive TTR]-[Fab “B”]]₂. (2) [Ab “A”]=[negative TTR]₂: [positive TTR]₂=[Ab “B”], and (3) [Ab “A”]=[negative TTR]₂: [[positive TTR]-[Fab “B”]]₂ Molecules (Co-Expression)

Denaturing LC-MS was conducted as described for the [Ab “A”]=[negative TTR]₂: [positive TTR]₂=[Ab “B”] Molecules.

SEC-Native-MS: all QToF experiments were performed on a Synapt G1 HDMS instrument operated in positive ESI mode. This instrument had been converted to an RF-confining drift-tube instrument, similar to that described by Bush et al., Anal Chem 2010, 82:9557-9565. All critical instrument voltages and pressures are as follows: capillary voltage 3.1 kV; sample cone 200 V, extraction cone 1 V; source block temperature 25° C.; trap collision energy 50 V; transfer collision energy 20 V; trap entrance 2.0 V; trap bias 5 V; trap exit 0.0 V; IMS entrance −20 V; IMS exit 21 V; transfer entrance 1.0V; transfer exit 1.0 V; transfer velocity 248 m/sec; transfer wave amplitude 3.0V; source RF-amplitude (peak-to-peak) 450V; triwave RF-amplitudes (peak-to-peak) trap 380V, IMS 250V, transfer 380V; source backing pressure 6.0 mbar; trap/transfer pressure cC4F8, 2.00e-2 mbar (pirani gauge indicated; flow rate 4.0 mL/min). Instrument control and data acquisition were carried out through MassLynx 4.1 SCN 872.

SEC was performed using an Agilent 1200 pump system and a 2.1×50 mm, 300A, Waters BEH operated at a flow rate of 75 μL/min at ambient temperatures. The mobile phase was 200 mM ammonium acetate. SEC separation was performed over an isocratic 6-min method. 25-50 μg of material was injected for analyses. 200 mM ammonium acetate was utilized since it is a volatile buffer, therefore compatible with a mass spectrometer. Instrument control was carried out through ChemStation.

The Following Techniques were Used to Generate TTR Negative and Positive Constructs Containing TTR Variants with Two TTR Dimer/Dimer Interface Mutations (“C10A/K15A/XX/YY”) Per TTR Subunit
Cloning of Fab TTR Diners with TTR Variants Containing Two TTR Dimer/Dimer Interface Mutations

The cloning of Fab TTR dimers with TTR variants containing two TTR dimer/dimer interface mutations was accomplished using analogous methods as those described in the section describing the cloning of (1) [Ab “A”]=[negative TTR]₂: [positive TTR]₂=[Ab “B”] (2× Ab-TTR); (2) [[Ab “A”]-[negative TTR]]₂: [[positive TTR]-[Ab “B”]]₂ (4× Ab-TTR); and (3) [[Fab “A”]-[negative TTR]]₂: [[positive TTR]-[Fab “B”]]₂ (4× Fab-TTR) Molecules (No Linker).

Expression of Fab TTR Dimers with TTR Variants Containing Two TTR Dimer/Dimer Interface Mutations

Transfections were carried out at 50 ml scale. HEK 293-6E cells were maintained in FreeStyle F17 Medium (Thermo Fisher Scientific) supplemented with 0.1% (w/v) Poloxamer 188 (Sigma-Aldrich), 6 mM L-Glutamine (Thermo Fisher Scientific), 25 μg/ml G418 (Thermo Fisher Scientific) at 36° C. in shaker flask in an incubator with 5% CO₂, 80%-90% humidity and 120 rpm agitation on a shaker of 25 mm shaking diameter. The 293-6E cells were seeded 2 days prior to transfection at 0.4×10⁶ cells/ml. On the day of transfection, the cells were at the exponential growth phase (˜1.5×10⁶ cells/ml, >95% viability). Transient transfections were performed by adding the mixture of 0.5 mg/L DNA and 2 mg/L PEI Max (Polyethylenimine Max, Polysciences, Cat #24765-2) to the cell culture. Proprietary feeds {Yeastolate (0.5% w/v) and glucose (3 g/L)} were added 4 hours post transfection. Productions were harvested 6 days post transfection by centrifuging cells at 4000 rpm (3485×g) for 40 minutes. The supernatant was filtered with 0.45 μM PES (polyethersulfone) filter.

Purification of Fab TTR Dimers with TTR Variants Containing Two TTR Dimer/Dimer Interface Mutations

Filtered cell culture media was injected onto a HisTrap excel column (GE Healthcare Bio-Sciences) and Desalting HiTrap column (GE Healthcare Bio-Sciences) in-line tandem purification system, equilibrated with 20 mM Na-phosphate, 500 mM NaCl, pH 7.4 and 10 mM MES 150 mM NaCl, pH 6.0, respectively, connected to an ÄKTApurifier (GE Healthcare Bio-Sciences). The HisTrap excel column was washed with 20 mM Na-phosphate, 500 mM NaCl, pH 7.4, and eluted stepwise with 20 mM Na-phosphate, 500 mM NaCl, 500 mM imidazole, pH 7.4. The HisTrap excel eluate was buffer exchanged on the Desalting HiTrap column with 10 mM MES, 150 mM NaCl, pH 6.0.

PC of Fab TTR Dimers with TTR Variants Containing Two TTR Dimer/Dimer Interface Mutations

Protein quantitation was performed by measuring UV absorbance at 280 nm using a MultiSkan FC Microplate Photometer (Thermo Fisher Scientific).

Non-reducing and reducing microcapillary electrophoresis analysis was performed on a Caliper LabChip GXII system using the Protein Express Assay LabChip (Perkin Elmer), per manufacturer protocol.

HPLC-SEC analysis was performed on an ACQUITY UPLC BEH450 SEC 2.5 μm 7.8×300 mm column (Waters Corp., Milford, Mass., USA) connected to an Agilent 1290 Infinity HPLC system (Agilent Technologies) running an isocratic 100 mM NaH2PO4, 50 mM NaCl, 7.5% EtOH, pH 6.9 mobile phase at 0.4 mL/min and observing UV absorbance at 280 nm.

### Heterodimerization of [[Fab “A”]-[Double Negative TTR]] and [[Double Positive TTR]-[Fab “B”]]: Mixing and Analysis of Separately Produced Constructs

Purified TTR-Fab samples were normalized to 0.2 mg/mL by dilution with 10 mM MES, 150 mM NaCl, pH 6.0. The samples were combined in equal volumes and incubated overnight at 4° C. The resulting molecule mixtures were analyzed by HPLC-SEC.

### Co-Expression of [[Fab “A”]-[Double Negative TTR]] and [[Double Positive TTR]-[Fab “B”]]

Transfections were carried out individually and, after completion (1-4 hours later), the [[Fab “A”]-[double negative TTR]] and [[double positive TTR]-[Fab “B”]] constructs were pooled and produced together at 4 ml scale. HEK 293-6E cells were maintained in FreeStyle F17 Medium (Thermo Fisher Scientific) supplemented with 0.1% (w/v) Poloxamer 188 (Sigma-Aldrich), 6 mM L-Glutamine (Thermo Fisher Scientific), 25 μg/ml G418 (Thermo Fisher Scientific) at 36° C. in shaker flask in an incubator with 5% CO2, 80%-90% humidity and 120 rpm agitation on a shaker of 25 mm shaking diameter. The 293-6E cells were seeded 2 days prior to transfection at 0.4×106 cells/ml. On the day of transfection, the cells were at the exponential growth phase (˜1.5×106 cells/ml, >95% viability). Transient transfections were performed by adding the mixture of 0.5 mg/L DNA and 2 mg/L PEI Max (Polyethylenimine Max, Polysciences, Cat #24765-2) to the cell culture. Proprietary feeds {Yeastolate (0.5% w/v) and glucose (3 g/L)} were added 4 hours post transfection. Productions were harvested 6 days post transfection by centrifuging cells at 4000 rpm (3485×g) for 40 minutes. The supernatant was filtered with 0.45 μM PES (polyethersulfone) filter.

### Purification of Co-Expressed [[Fab “A”]-[Double Negative TTR]] and [[Double Positive TTR]-[Fab “B”]]

The filtered cell culture media was injected onto a HisTrap excel column (GE Healthcare Bio-Sciences) and Desalting HiTrap column (GE Healthcare Bio-Sciences) in-line tandem purification system, equilibrated with 20 mM Na-phosphate, 500 mM NaCl, pH 7.4 and 10 mM MES 150 mM NaCl, pH 6.0, respectively, connected to an ÄKTApurifier (GE Healthcare Bio-Sciences). The HisTrap excel column was washed with 20 mM Na-phosphate, 500 mM NaCl, pH 7.4, and eluted stepwise with 20 mM Na-phosphate, 500 mM NaCl, 500 mM imidazole, pH 7.4. The HisTrap excel eluate was buffer exchanged on the Desalting HiTrap column with 10 mM MES, 150 mM NaCl, pH 6.0.

### PC Analytics of Co-Expressed [[Fab “A”]-[Double Negative TTR]] and [[Double Positive TTR]-[Fab “B”]]

Protein quantitation was performed by measuring UV absorbance at 280 nm using a MultiSkan FC Microplate Photometer (Thermo Fisher Scientific). Non-reducing and reducing microcapillary electrophoresis analysis was performed on a Caliper LabChip GXII system using the Protein Express Assay LabChip (Perkin Elmer), per manufacturer protocol. HPLC-SEC analysis was performed on an ACQUITY UPLC BEH450 SEC 2.5 μm 7.8×150 mm column (Waters Corp., Milford, Mass., USA) connected to an Agilent 1290 Infinity HPLC system (Agilent Technologies) running an isocratic 100 mM NaH2PO4, 50 mM NaCl, 7.5% EtOH, pH 6.9 mobile phase at 0.4 mL/min and observing UV absorbance at 280 nm.

### Example 2: Evaluation of TTR Heterotetramers Comprising TTR Variants with One TTR Dimer/Dimer Interface Mutation (“C10A/K15A/XX”) Per TTR Subunit—Produced in e. coli

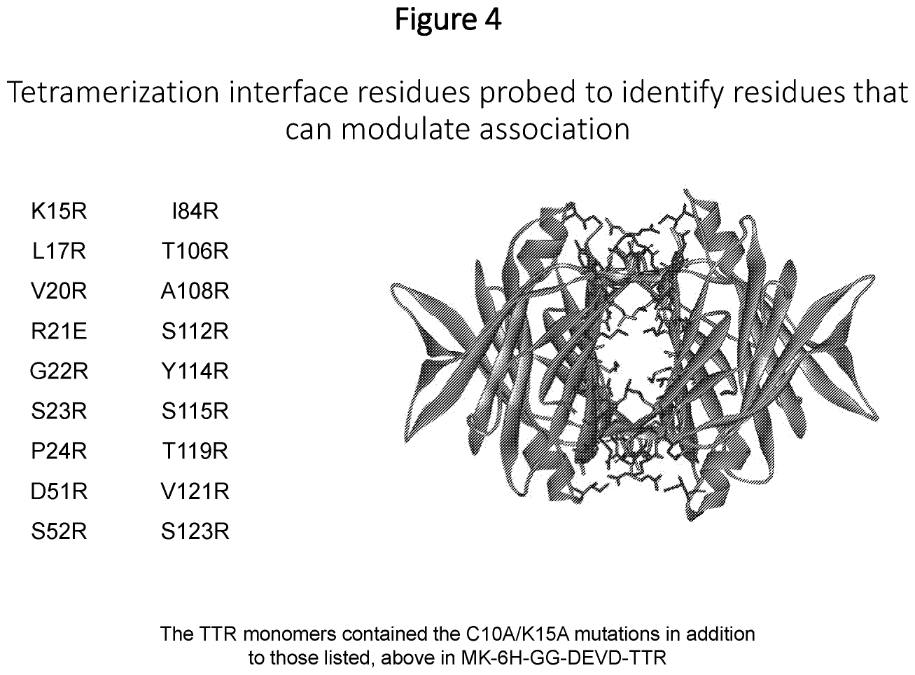

18 TTR charge variants (C10A/K15A/XX) of TTR (SEQ ID NO: 1) were made to determine which charge mutations would result in substantial repulsion of the TTR dimer/dimer interface (See FIG. 4). Each of the TTR variants contained the C10A and K15A mutations and a third mutation, denoted as “XX.” In these experiments, XX was K15R, L17R, V20R, R21E, G22R, S23R, P24R, D51R, S52R, 184R, T106R, A108R, S112R, Y114R, S115R, T119R, V121R, or S123R.

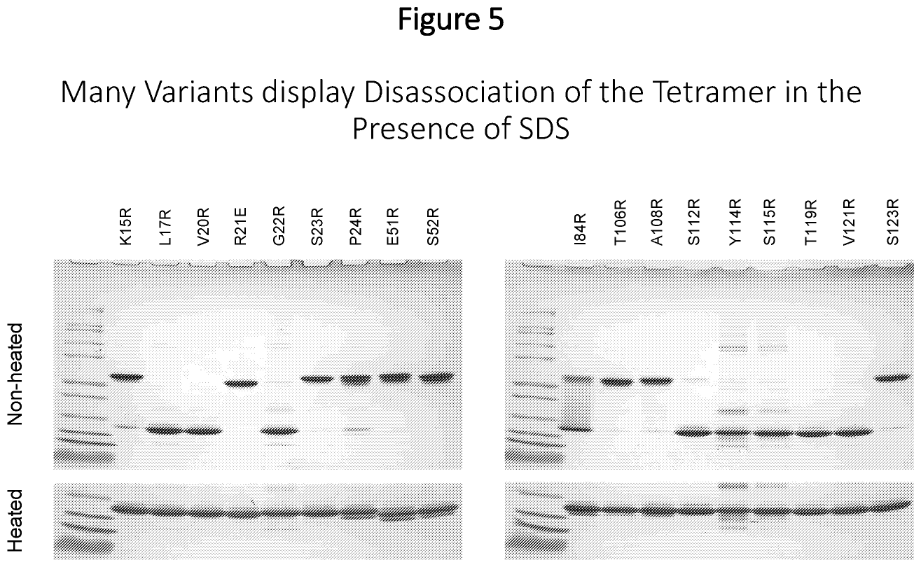

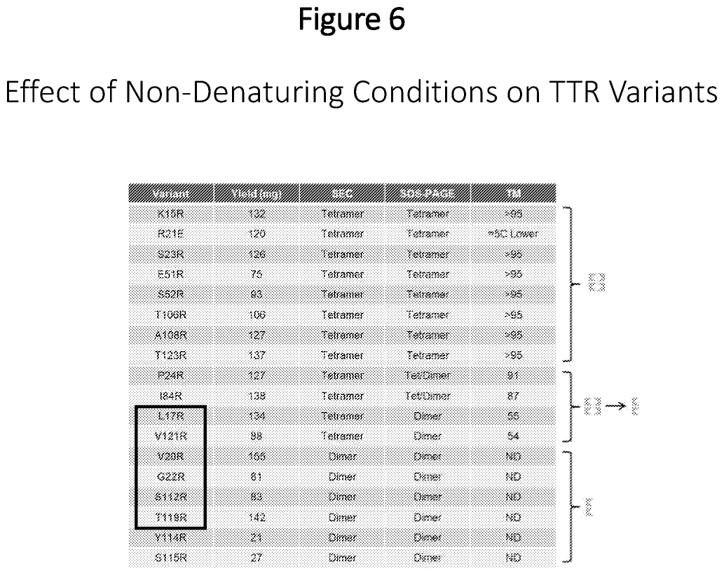

Nine of the TTR variants (P24R, I84R, L17R, V121R, V20R, G22R S112R, T119R, Y114R, and S115R) showed substantial weakening of the TTR dimer/dimer interface as evidence by the fact that the TTR tetramer was broken into the corresponding TTR dimers in the presence of SDS (chaotrope) without heating (See FIG. 5, “Non-heated” gels). Six of the variants (V20R, G22R, S112R, T119R, Y114R, and S115R) also interfere with the dimer/dimer interaction under non-denaturing (native) conditions as assessed by SEC (See FIG. 6). In addition, four of the variants (P24R, I84R, L17R, and V121R) were observed to mostly form TTR tetramers under non-denaturing SEC conditions and showed lower melting temperatures, again indicating the dimer/dimer interface was weakened in those variants.

The most favored six dimer/dimer interface mutation sites (FIG. 6, in red) were chosen to generate additional TTR variants to evaluate the formation of TTR heterotetramers comprising two different TTR monomer sequences. In these experiments, the desired TTR heterotetramers comprise [1] one TTR dimer which is itself comprised of two TTR monomers, each monomer being a “negative” TTR variant; and [2] one TTR dimer which is itself comprised of two TTR monomers, each monomer being a “positive” TTR variant. The favored six dimer/dimer interface mutation sites were selected based on their ability to form heterotetramers under SEC and SDS-PAGE conditions. Mutation sites which led to primarily dimer formation under SDS-PAGE conditions were selected as an initial cutoff (i.e., L17R, V121R, V20R, G22R, S112R, T119R, Y114R, and S115R). Y114R and S115R were removed from further consideration due to an apparently low yield of protein.

The negative TTR variants contained the C10A/K15A/XX mutations, wherein each XX was L17D, L17E, V20D, V20E, G22D, G22E, S112D, S112E, T119D, T119E, V121D, or V121E. Similarly, the positive TTR variants contained the C10A/K15A/XX mutations, wherein each XX was L17R, L17K, V20R, V20K, G22R, G22K, S112R, S112K, T119R, T119K, V121R, or V121K. Thus, a total of 24 charge interface variants (12 negative and 12 positive) were produced (FIG. 7).

The positive variants were mixed with negative variants (in pairwise fashion) and assessed for TTR heterotetramer formation by both SDS-PAGE and SEC. Many of the variant pairings showed some propensity to form desirable heterotetramer as indicated by a non-zero SEC value in FIG. 7 (with the value representing the % of heterotetramer formation). Indeed, some pairings showed a very high propensity to form heterotetramer with 40-100% tetramer formation.

Many of these TTR heterotetramer were resistant to breakdown by the chaotrope SDS as indicated by the SDS-PAGE results, also shown in FIG. 7. Positive/negative pairings that demonstrated a high propensity to form stable heterotetramer (cross-referencing SEC and SDS-PAGE data) include: L17R/T119D. L17K/T119D, L17K/V121E, V20R/V20D, V20R/V20E, V20K/V20D, V20K/V20E, V121R/L17D, V121R/L17E, and V121K/L17D.

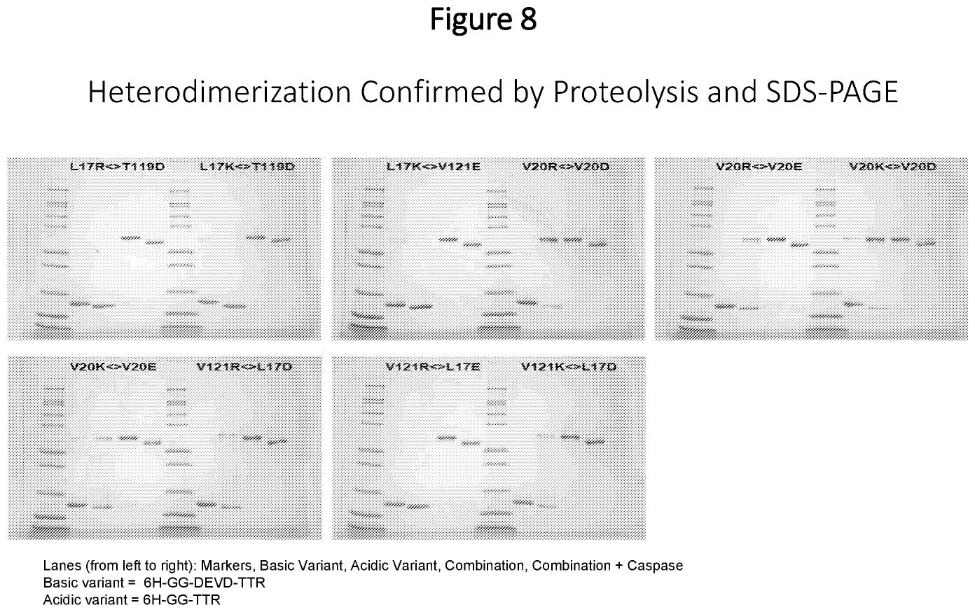

The positive/negative pairings that demonstrated a high propensity to form stable TTR tetramers were further assessed by SDS-PAGE. In this experiment, for each pairing, [1] the negative (i.e., basic) variant; [2] the positive (i.e., acidic) variant; [3] the combination of negative and positive variants (which should form a tetramer); and [4] the combination of negative and positive variants exposed to caspase, were evaluated (FIG. 8). As demonstrated in FIG. 8, the separate negative and positive variants migrated to the bottom of the gel, while the combination of the negative and positive variants migrated only part way down the gel. This further demonstrates that the negative and positive variants, upon combination, form a high molecular weight species (HMW)—likely the desired heterotetramer. Treatment of the combination of negative and basic variants with caspase (which only cleaves the poly-histidine tag from the positive variants due to the inclusion of the DEVD sequence) produces a mostly uniform band that runs slightly lower than the uncleaved tetramer, demonstrating that the positive component is present and showing the tetramers are mostly uniform and likely heterotetramers.

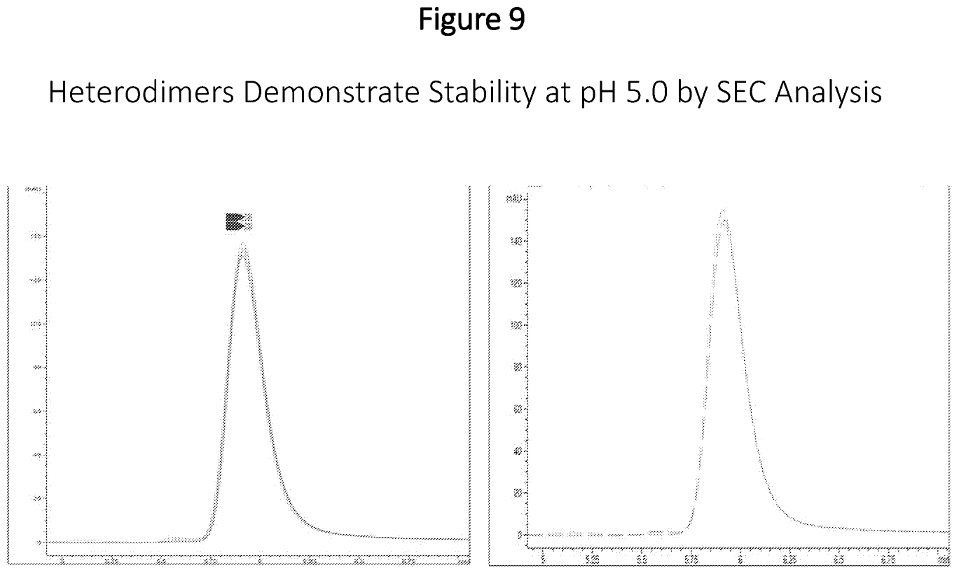

Heterotetramers comprising the L17R/T119D, L17K/T119D, L17K/V121E, V20R/V20D, V20R/V20E, V20K/V20D, V20K/V20E, V121R/L17D, V121R/L17E, and V121K/L17D pairings were then exposed to pH 5.0 conditions to determine whether they could maintain their tetrameric state (via SEC) in conditions similar to those found in pharmaceutical formulations (FIG. 9). Indeed, the single peak in FIG. 9 indicates that the heterotetramers were able to maintain their tetrameric state.

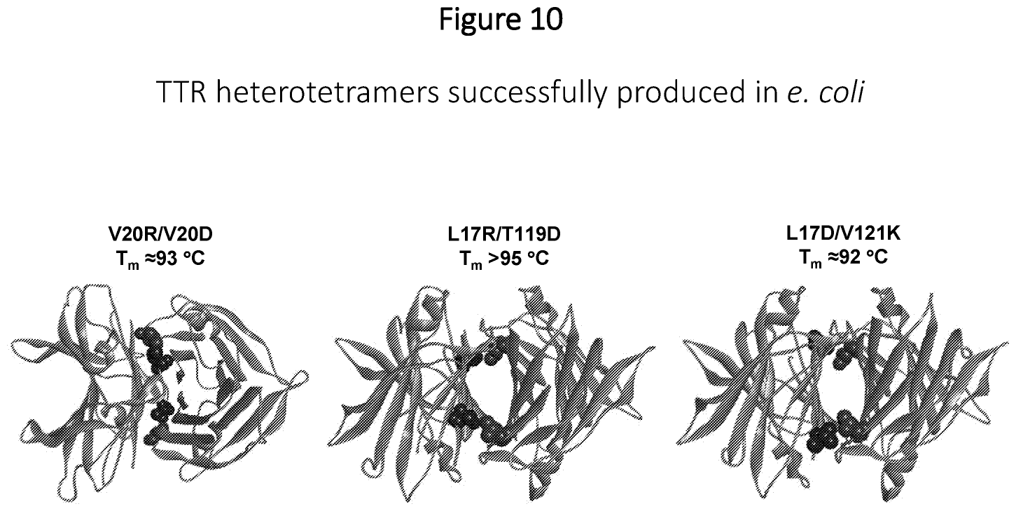

The melting temperature of three heterotetramers was assessed. In each case, the heterotetramer was stable to at least 92° C. indicating the heterotetramer is very thermally stable (FIG. 10).

### Example 3: Evaluation of TTR Heterotetramer Fab, Ab, and Mixed Fab/Ab Constructs Comprising TTR Variants with One TTR Dimer/Dimer Interface Mutation (“C10A/K15A/XX”) Per TTR Subunit—Produced in Mammalian Cells

The ability to form TTR heterotetramer Fab, Ab, and mixed Fab/Ab constructs was evaluated. In these constructs, TTR tetramers comprising two positive TTR variants and two negative TTR variants (as described above) can be used to generate TTR heterotetramers attached to four Fabs, 2 Abs, or 1 Ab and 2 Fabs. See FIGS. 2a, 2b, and 2c, respectively. Charge pair mutations in the Fab constructs can be used to drive proper HC/LC Fab pairings. One benefit of such TTR heterotetramer constructs is that they allow for the assembly of multiple antigen targeting moieties (e.g., Abs and/or Fabs) by fusion of the TTR monomeric units to the C-terminus of the Ab and/or Fab. This orients the Abs and/or Fabs such that their antigen binding domains are unlikely to suffer the steric interference seen in other bivalent bispecific platforms (e.g., IgG-Fab and IgG scFv constructs), typically due to fusion of the Fab or scFv N-terminus to the IgG C-terminus.

### TTR Heterotetramer Ab Constructs

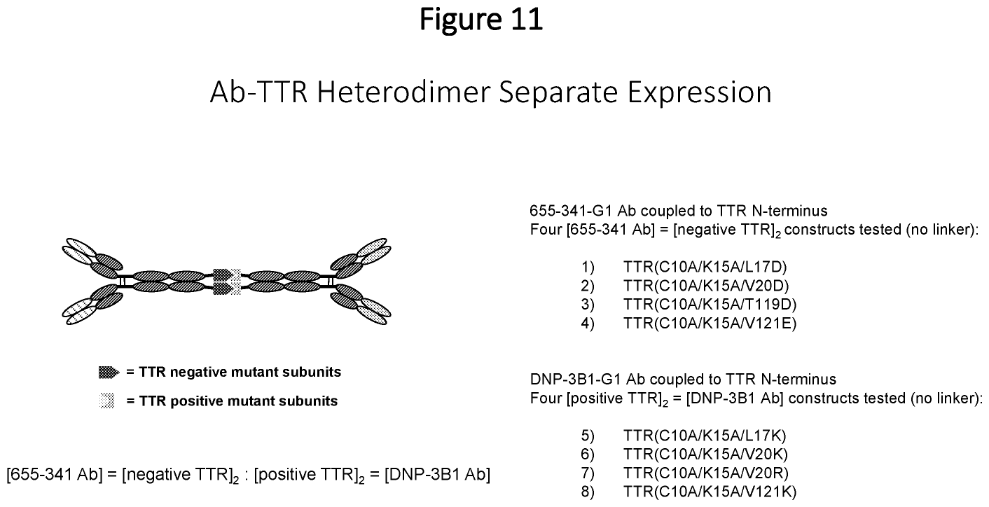

Bispecific TTR heterotetramer Ab constructs were generated. In these constructs, one Ab (655-341 Ab) was specific for the extracellular domain of human TRAIL (Tumor Necrosis Factor-Related Apoptosis-Inducing Ligand) Receptor 2 (TR-2, death receptor 5), while the other Ab (DNP-3B1) was specific for DNP. An exemplary bispecific TTR heterotetramer Ab construct is shown in FIG. 11 wherein each heavy chain of the 655-341 Ab (lined fill) attached to the N-terminus of the negative TTR monomers (together forming a negative TTR dimer) and each heavy chain of the DNP-3B1 Ab (solid fill) attached to the N-terminus of the positive TTR monomers (together forming a positive TTR dimer).

Four negative TTR variants were fused to the 655-341 Ab and four positive TTR variants were fused to the DNP-3B1 Ab (FIG. 11). All Ab-TTR fusions were made without a linker between the Ab and the TTR monomer. These variants were produced in mammalian cells (293-6E HEK cells) for secretion into the medium. Two sets of transformed cells were generated: one producing the 655-341 Ab/negative TTR fusion ([655-341 Ab]=[negative TTR]₂), and one producing the DNP-3B1 Ab/positive TTR fusion ([positive TTR]₂=[DNP-3B1 Ab]).

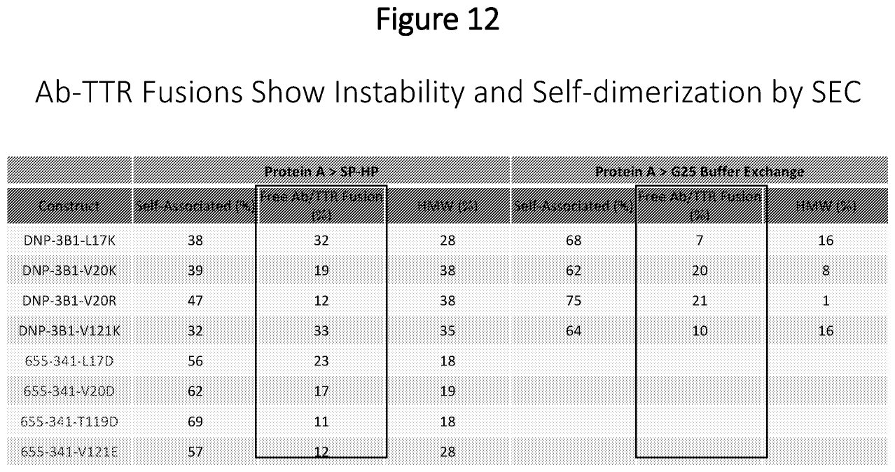

Interestingly, and in contrast to the TTR tetramers produced in e. coli (Example 2), a significant amount of secreted constructs were: self-associated 655-341 Ab/negative TTR fusions (i.e., [655-341 Ab]=[negative TTR]₂: [negative TTR]₂=[655-341 Ab]), self-associated DNP-3B1 Ab/positive TTR fusions (i.e., [DNP-3B1 Ab]=[positive TTR]₂: [positive TTR]₂=[DNP-3B1 Ab]), or other HMW species. The free 655-341 Ab/negative TTR fusions ([655-341 Ab]=[negative TTR]₂) and DNP-3B1 Ab/positive TTR fusions ([positive TTR]₂=[DNP-3B1 Ab]) accounted for 11-32% of the secreted constructs (FIG. 12). Rapid buffer exchange reduced the amount of HMW species, though this led primarily to an increase in self-associated species.

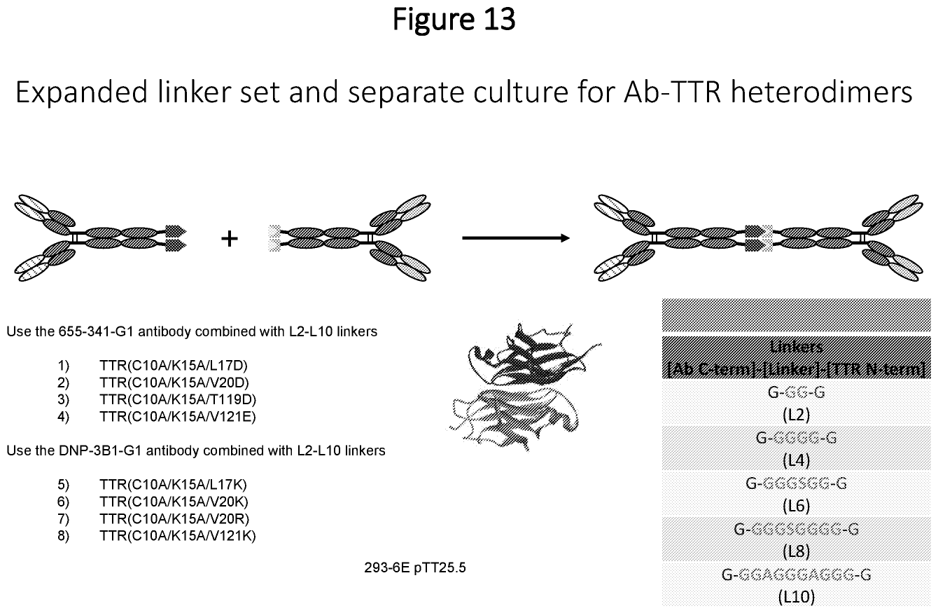

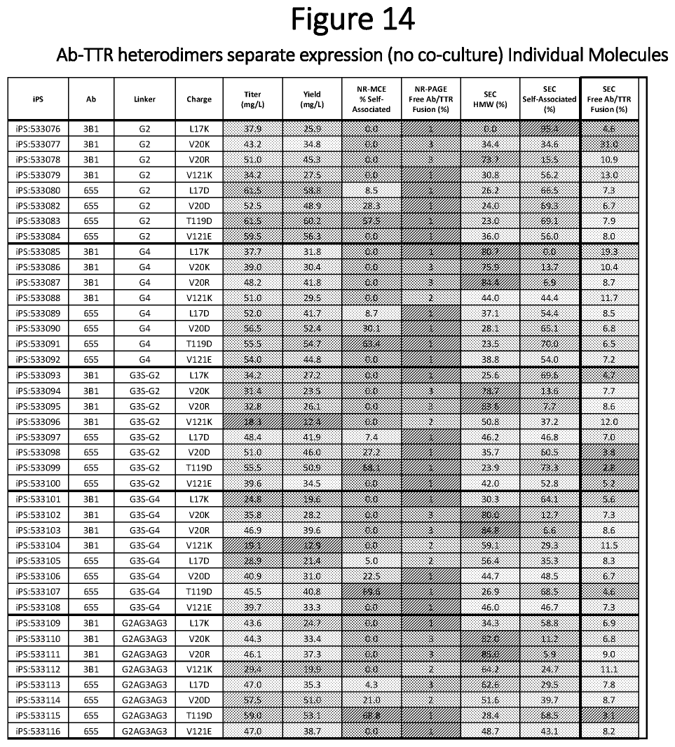

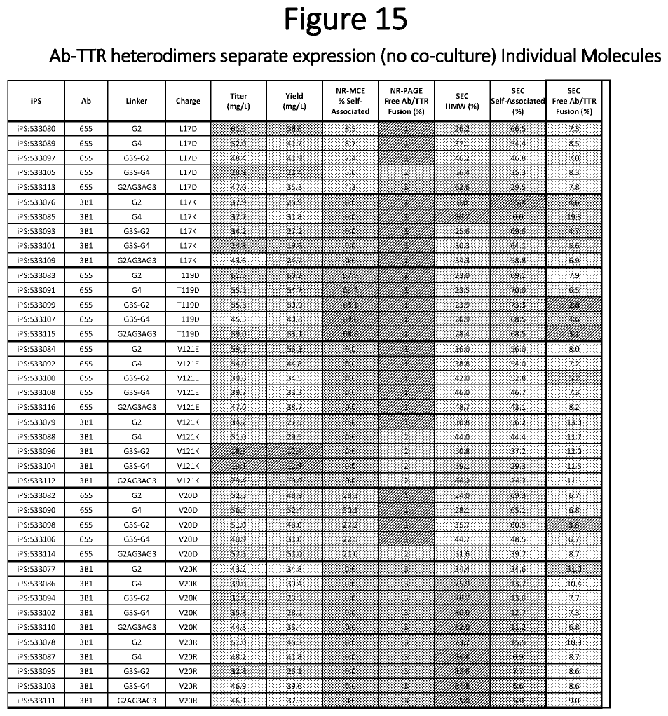

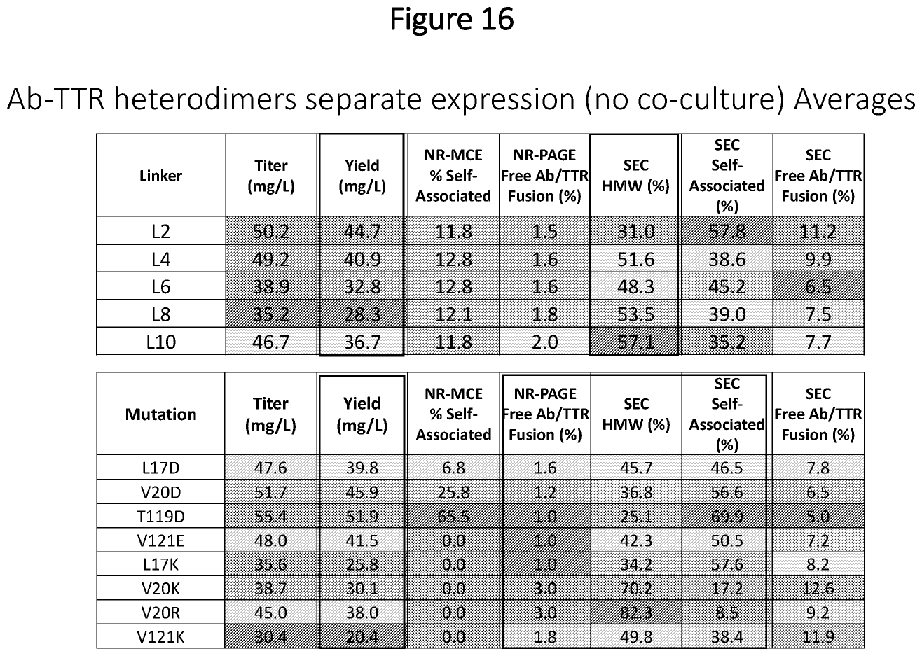

The effect of adding a linker between the Ab heavy chain and the TTR monomer was investigated using the same eight charge variants. Five linkers ranging in size from 2-10 amino acids were evaluated (FIG. 13). The predominantly secreted linker-containing Ab/TTR fusion species were again self-associated (e.g., [655-341 Ab]=[[LX]: [negative TTR]]₂: [[negative TTR]: [LX]]₂=[655-341 Ab]) and HMW species. The free 655-341 Ab/negative TTR fusions and DNP-3B1 Ab/positive TTR fusions ([655-341 Ab]=[[LX]: [negative TTR]]₂ and [[positive TTR]: [LX]]₂=[DNP-3B1 Ab]) accounted for up to 31% of the secreted constructs (FIGS. 14 and 15). Notably, shorter linkers generally resulted in higher titers and yield (i.e., more protein production) and lower levels of HMW species (FIG. 16). In addition, the negative TTR fusions yielded modestly higher yields and lower HMW species levels. None of the linker-containing Ab-TTR fusions formed free 655-341 Ab/negative TTR fusion or DNP-3B1 Ab/positive TTR fusion as the predominant product. Other observations included that shorter linkers produced higher levels of SDS-resistant self-associated Ab/TTR fusions; V20K, V20R, and V121K produced higher levels of SDS-susceptible self-associated Ab/TTR fusions; T119D, V20D, V121E, and L17D produced fusions with higher titers/yields and reduced amounts of free Ab/TTR fusions; and negative variants appeared to yield lower amounts of HMW species.

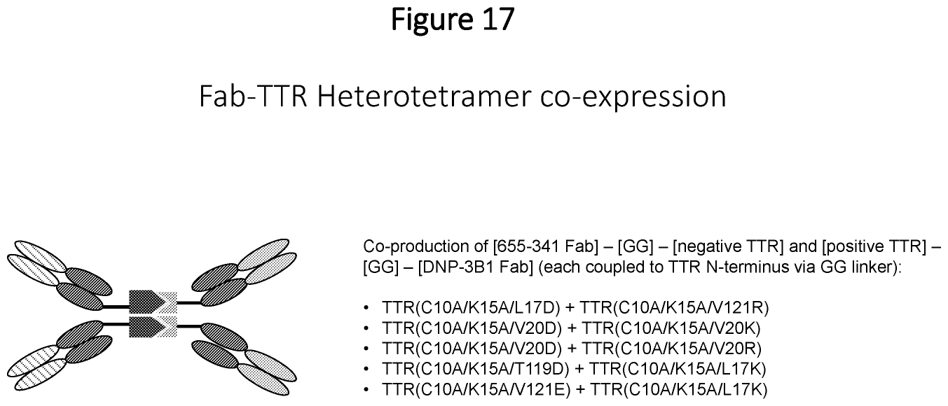

In light of these observations, it was hypothesized that mammalian cells may not efficiently produce “untetramerized” TTR. That is, mammalian cells may have difficulty producing [655-341 Ab]=[negative TTR]₂ or [positive TTR]₂=[DNP-3B1 Ab] (with or without linkers) because such constructs contain only two TTR monomers (compared to naturally occurring TTR which has four TRR monomers). To investigate this, five combinations of 655-341 Fab/negative TTR fusions and DNP-3B1 Fab/positive TTR fusions were co-produced in a mammalian cell line (CHO K1). In these constructs, the Fab HC was attached to TTR via a GG linker (FIG. 17).

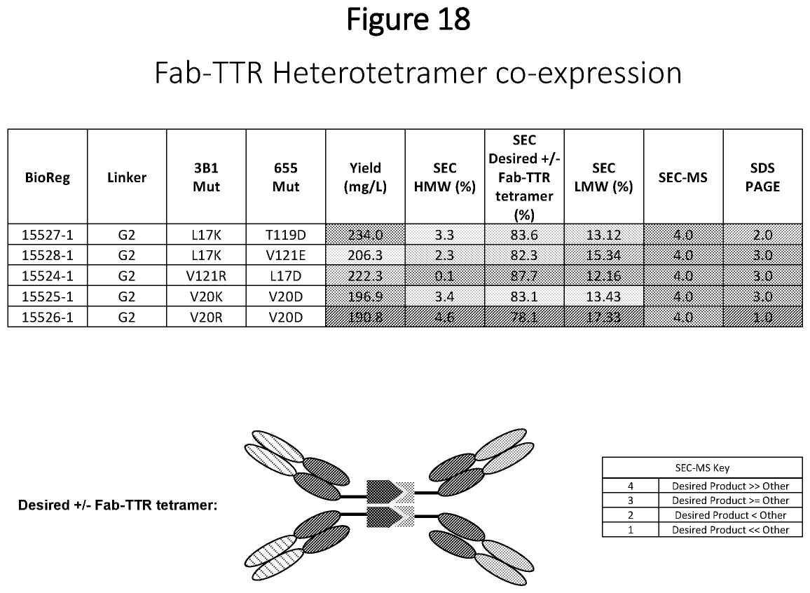

Co-production of the [655-341 Fab]-[GG]-[negative TTR] and [positive TTR]-[GG]-[DNP-3B1 Fab] in CHO K1 led to significant formation of the desired [[655-341 Fab]-[GG]-[negative TTR]]₂: [[positive TTR]-[GG]-[DNP-3B1 Fab]]₂ (78-88%) and a reduced amount of the HMW species (in each case below 5%) (FIG. 18). Fab formation was found to be very efficient for all mutation pairs, though there appeared to be a slight benefit with the L17D/V121R paid. In addition, it appeared as if the V20R/V20D pair may form a weaker heterotetramer that can be disrupted by SDS.

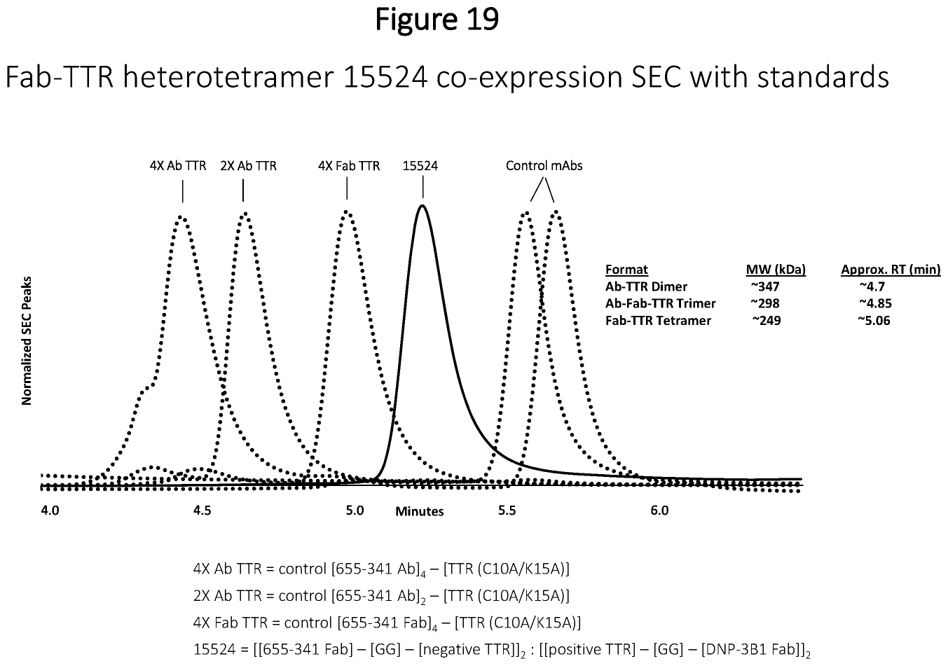

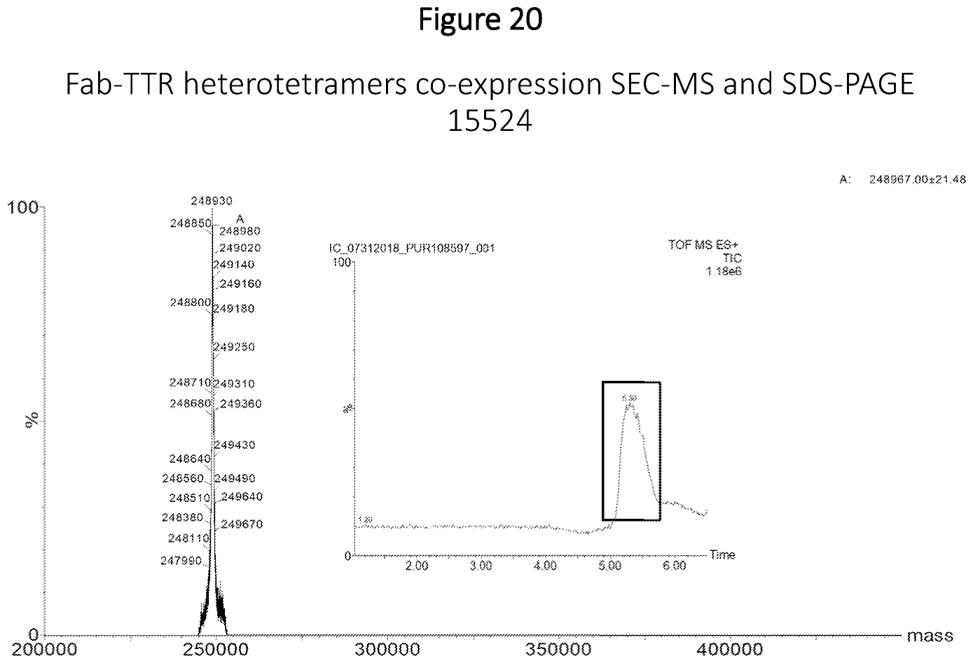

Using Ab- and Fab-TTR fusions as well as unfused Abs as SEC standards, it can be seen that molecule 15524 ([655-341 Fab]-[GG]-[TTR(C10A/K15A/L17D)] and [TTR(C10A/K15A/V121R)]-[GG]-[DNP-3B1 Fab]) has a retention time similar to that of the 4×-Fab homomultimer, and substantially shorter than that expected for a 2×-Fab-TTR fusion (which should elute after the control Abs) (FIG. 19). Furthermore, when 15524 is assessed by SEC coupled MS, the molecular mass of the eluting species is consistent with that expected (FIG. 20).

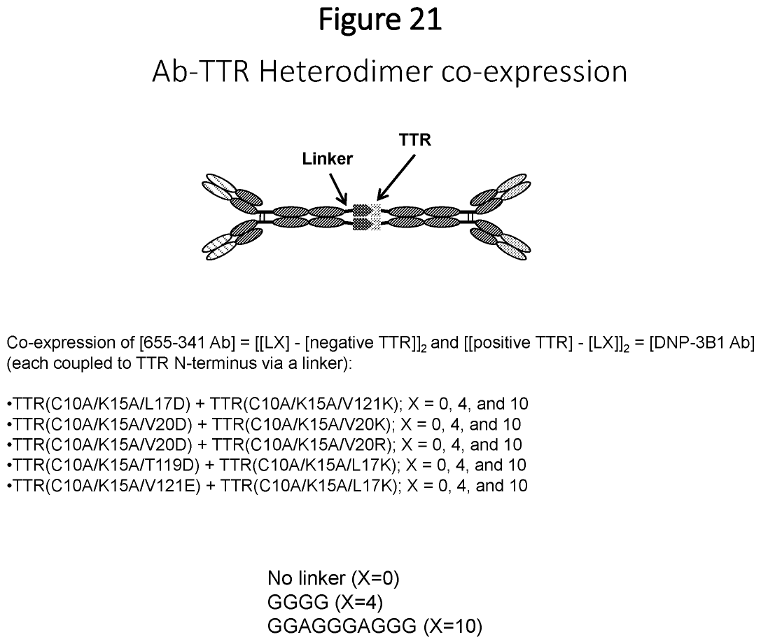

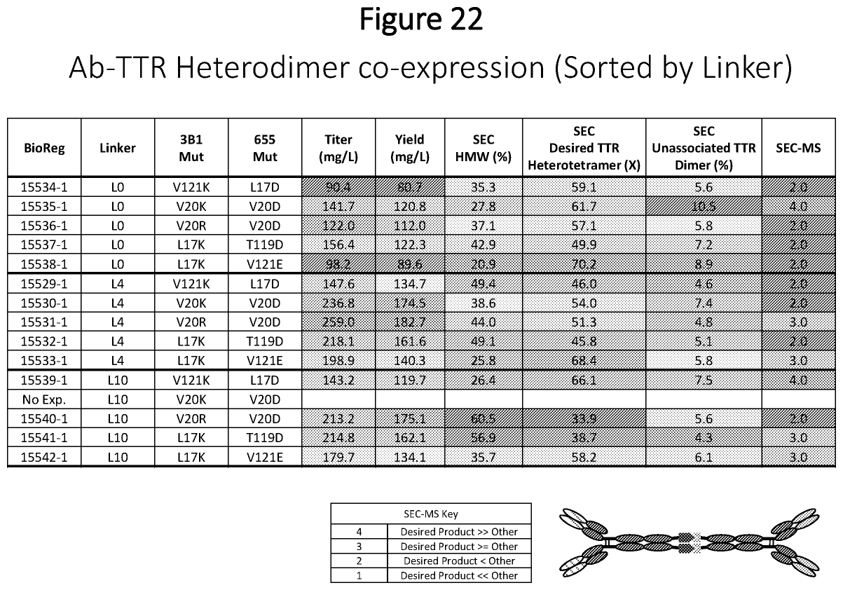

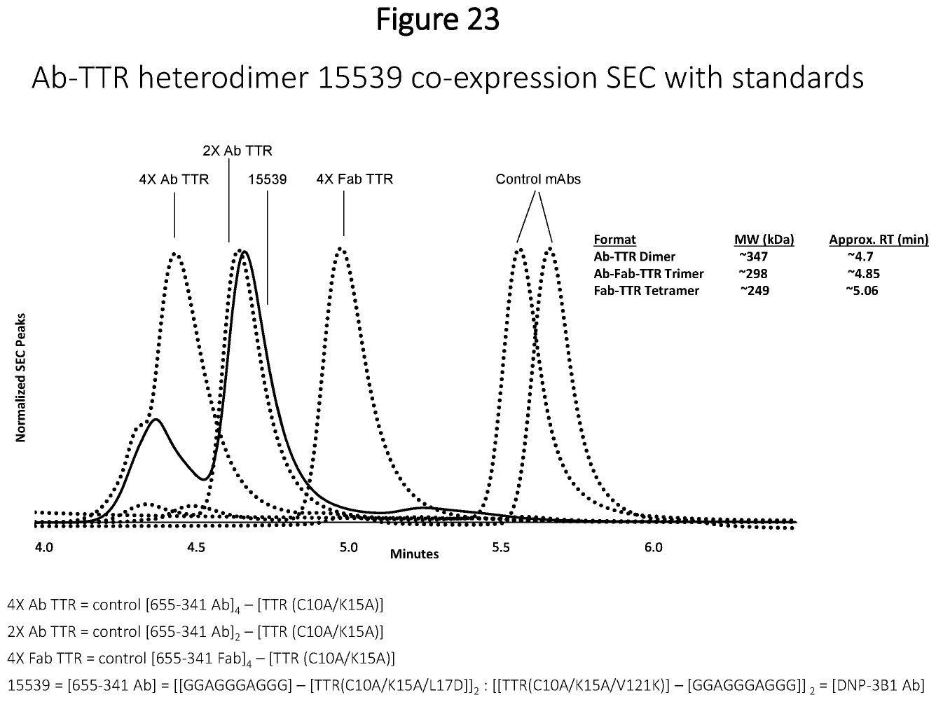

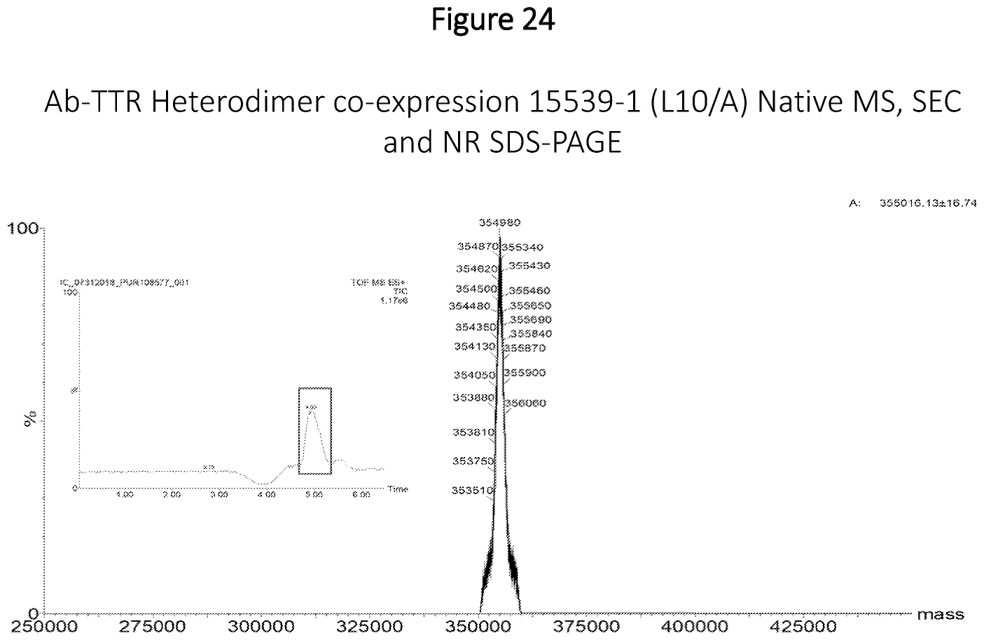

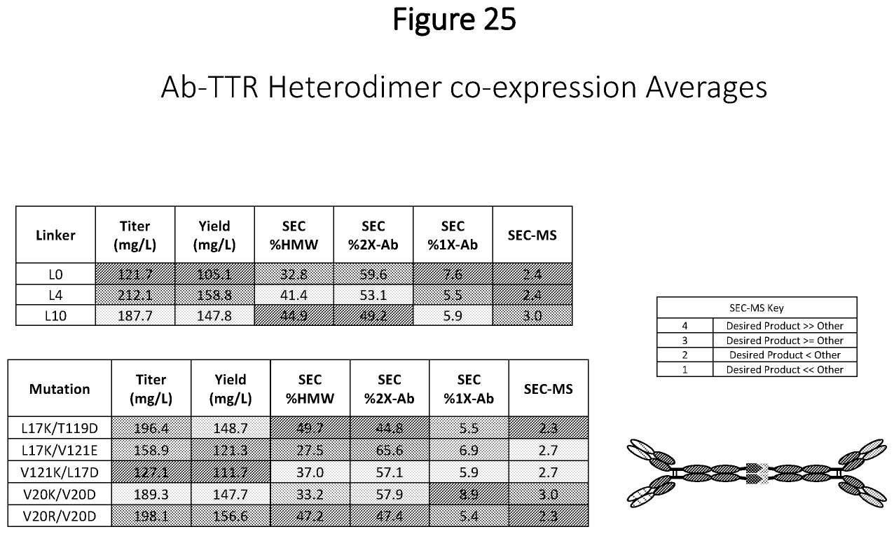

The same approach was undertaken to determine if co-expression of Ab-containing TTR fusions would lead to the generation of desired TTR heterotetramer [655-341 Ab]=[[LX]-[negative TTR]]₂: [[positive TTR]-[LX]]₂=[DNP-3B1 Ab]. A total of five TTR charge variants and three linker lengths (X=0, 4, and 10 amino acids), for a total of 15 combinations, were tested (FIG. 21). A significant amount of the desired TTR heterotetramer was formed for many of the combinations, up to 70% (FIG. 22). In addition, the 4 amino acid linker yielded higher titers and yields, compared to the 0 amino acid linker. Using Ab- and Fab-TTR fusions as well as unfused Abs as SEC standards, it can be seen that molecule 15539 ([655-341 Ab]=[[GGAGGGAGGG]-[TTR(C10A/K15A/L17D]]₂: [[TTR(C10A/K15A/V121K)]-[GGAGGGAGGG]]₂=[DNP-3B1 Ab]) was observed to have a main peak retention time nearly identical to that of the 2×-Ab-TTR homomultimer (FIG. 23). Furthermore, when the construct was assessed by SEC coupled MS, the molecular mass of the eluting species is consistent with that of the desired TTR heterotetramer (FIG. 24). It was observed that, on average, the L4 linker appears to lead to a good yield coupled with good preferential production of the desired product. In addition, the L17K/T119D, V20K/V20D, and V20R/V20D mutation combinations appeared to lead to high expression and yield. Finally, the L17K/V121E, V121K/L17D, and V20K/V20D mutation combinations appeared to be best able to drive desired assembly of the TTR heterotetramer. See, FIG. 25.

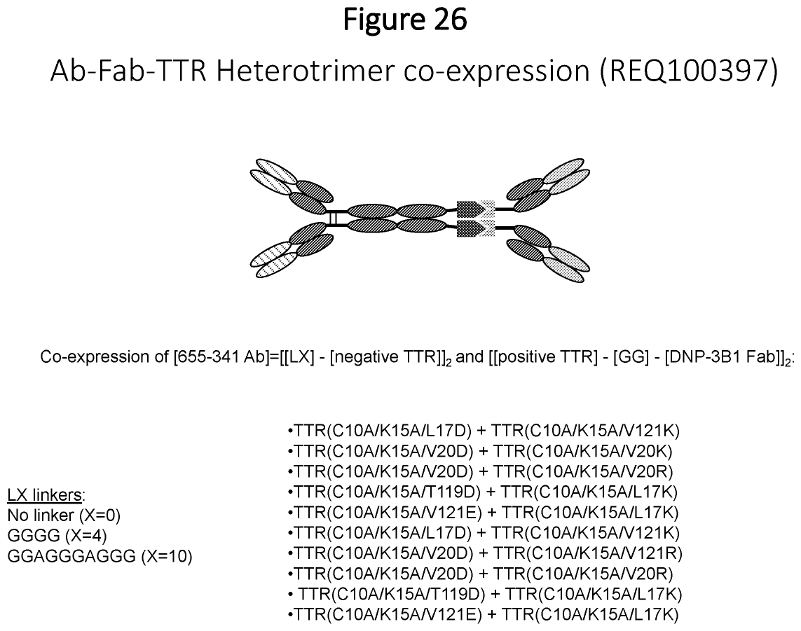

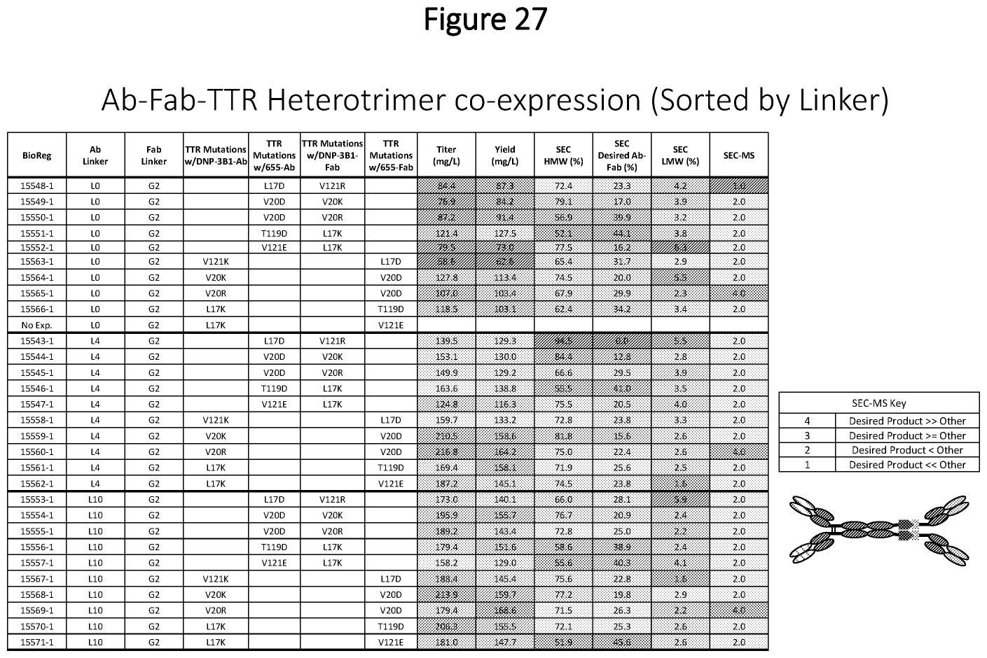

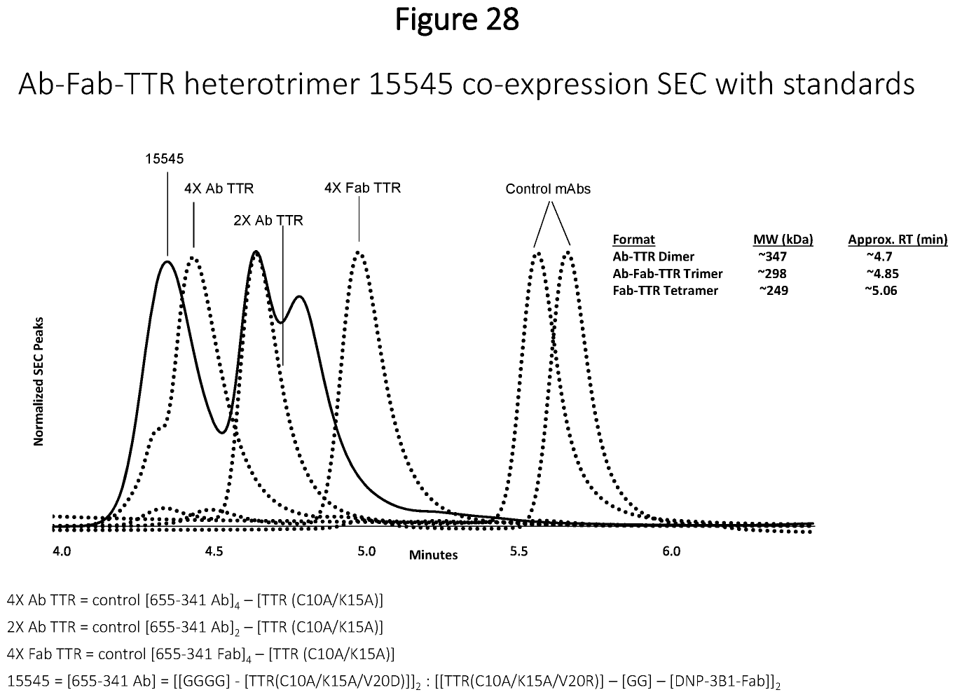

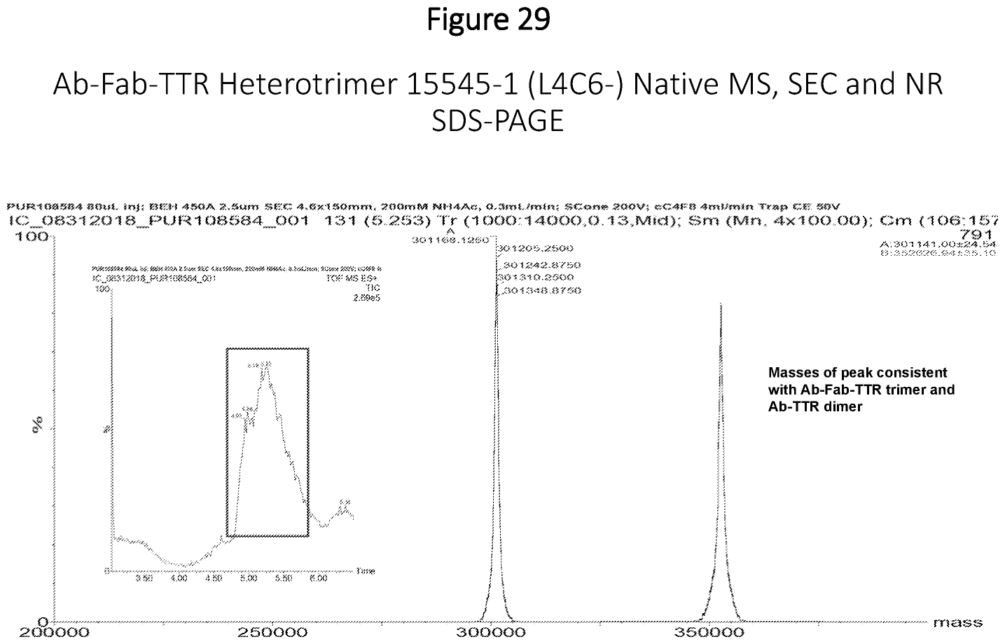

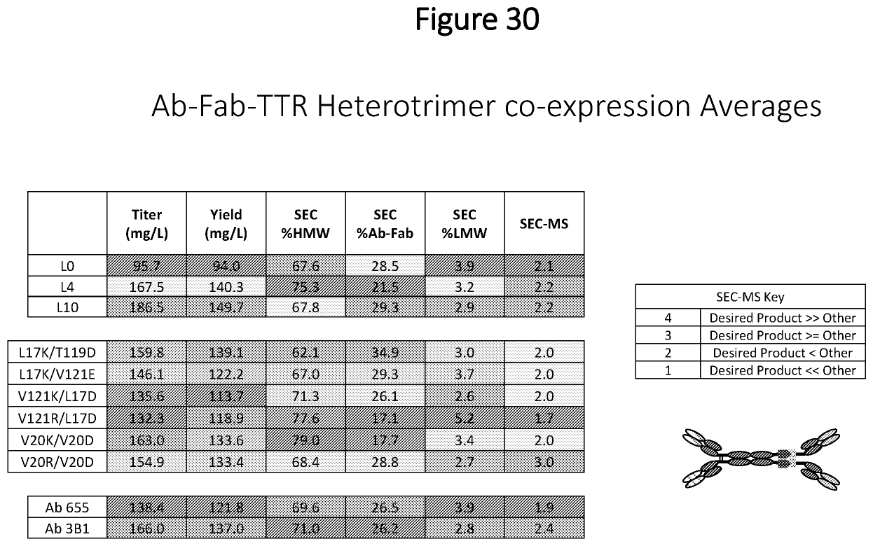

We next sought to determine if co-expression of the negative and positive TTR variants (4 of each mutation) fused to Abs and Fabs in the same cell line would lead to the production of Ab-Fab-TTR constructs (i.e., [Ab “A”]=[negative TTR]₂: [[positive TTR]=[Fab “B”]]₂ constructs). A total of five TTR variants in combination with three linker lengths (X=0, 4, and 10 amino acids) and two Abs/Fabs—for a total of 30 combinations—were tested (FIG. 26). The quantity of Ab fusions that formed the desired Ab-Fab-TTR construct based on SEC for many of the combinations was up to 45.6% (FIG. 27). In addition, the 4 amino acid and 10 amino acid linkers appear to yield more of the desired Ab-Fab-TTR construct compared to the 0 amino acid linker (based on titer and purified yield). Using Ab- and Fab-TTR fusions as well as unfused Ab as SEC standards, it was observed that molecule 15545 ([655-341 Ab]=[[GGGG]-[TTR(C10A/K15A/V20D)]]₂: [[TTR(C10A/K15A/V20R)]-[GG]-[DNP-3B1-Fab]]₂) produced a peak with a retention between the 2×-Ab-TTR homomultimer and 4×-Fab-TTR homomultimer as would be expected (FIG. 28). Furthermore, when this molecule is assessed by SEC coupled MS, the molecular mass of an eluting species is consistent with the desired construct (FIG. 29). It was observed that, on average, the L10 linker was preferred in the Ab-Fab-TTR construct. In addition, it appears the L17K/T119D TTR mutations are preferred in the Ab-Fab-TTR construct. See, FIG. 30.

### Example 4: Evaluation of TTR Heterotetramer Fab, Ab, and Mixed Fab/Ab Constructs Comprising TTR Variants with Two TTR Dimer/Dimer Interface Mutations (“C10A/K15A/XX/YY”) Per TTR Subunit—in Mammalian Cells

Ab- and Fab-TTR fusions with two charged interface mutations were produced separately in mammalian cells as previously described for the single charge interface mutations. The titer, post-affinity chromatography yield and SEC performance were evaluated as previously described for the single charge interface mutations. The purified single and double charged interface variants were then mixed in a matrix fashion and the mixtures were assessed by SEC as previously described for the single charge variants.

Ab- and Fab-TTR fusions with single and double charged interface mutations were produced by co-culturing mammalian cells with oppositely charged TTR variants as previously described for the single charge interface mutations. The post-affinity chromatography yield and SEC performance were evaluated as previously described for the single charge interface mutations.

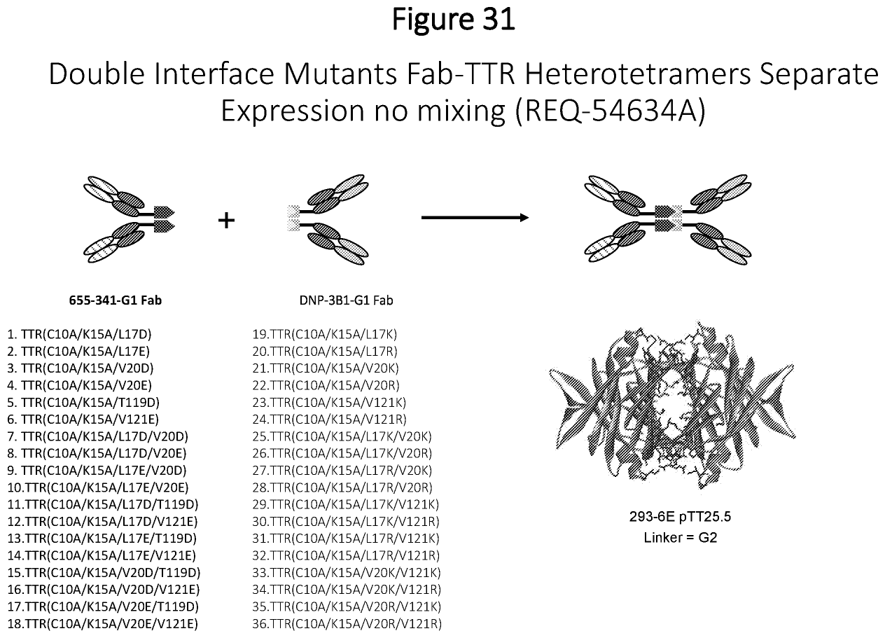

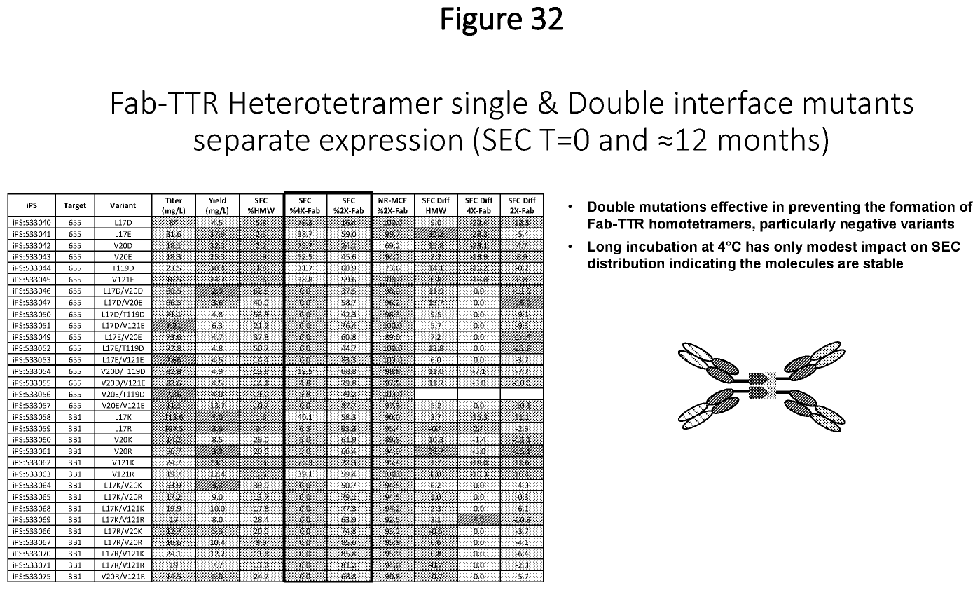

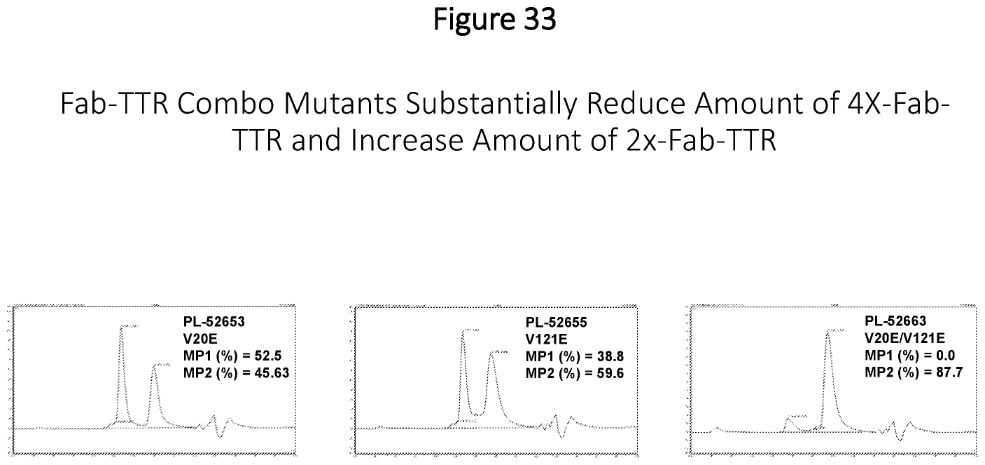

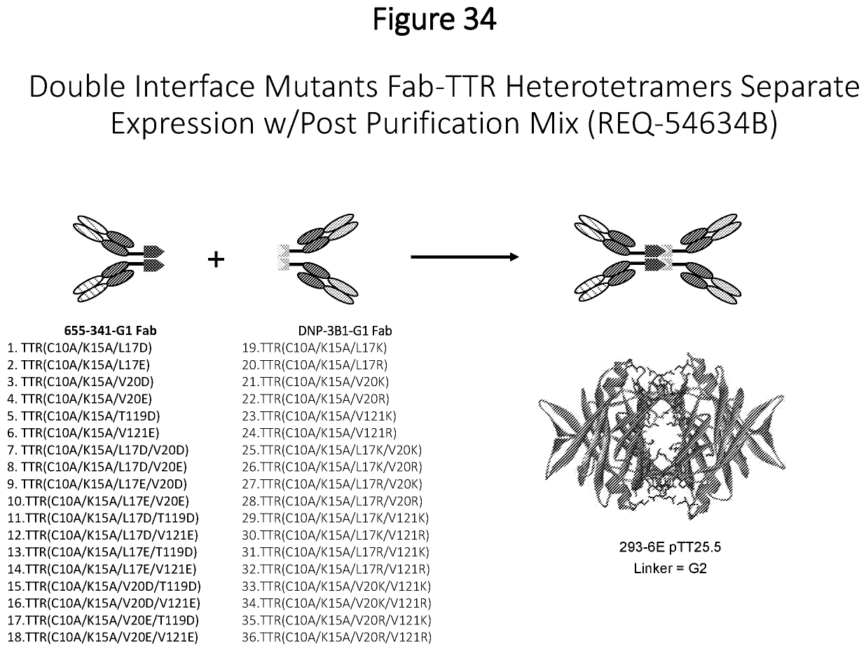

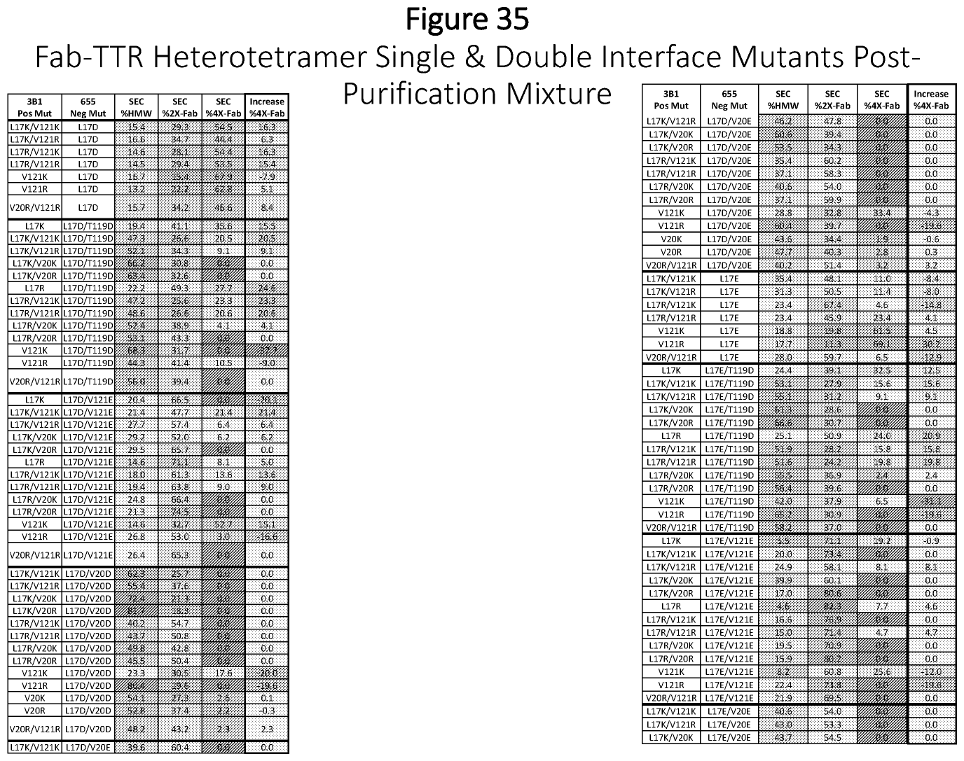

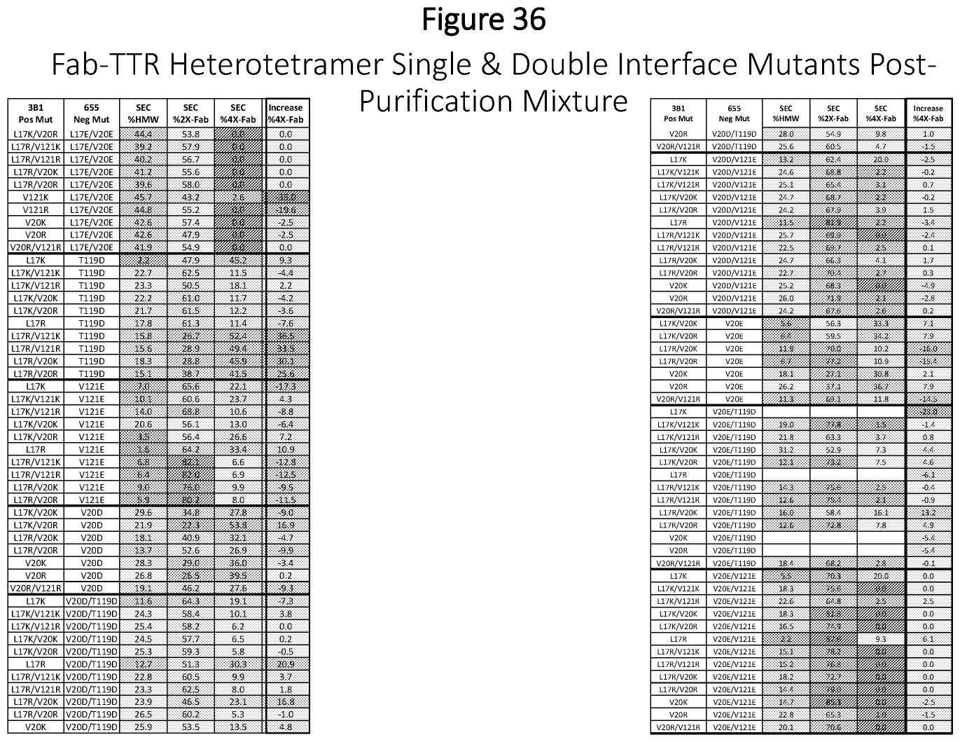

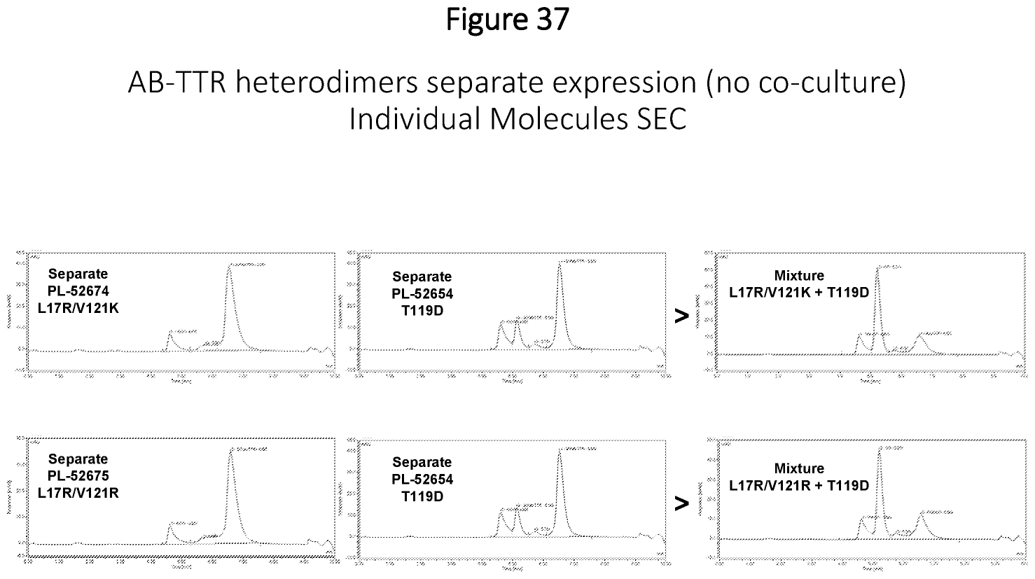

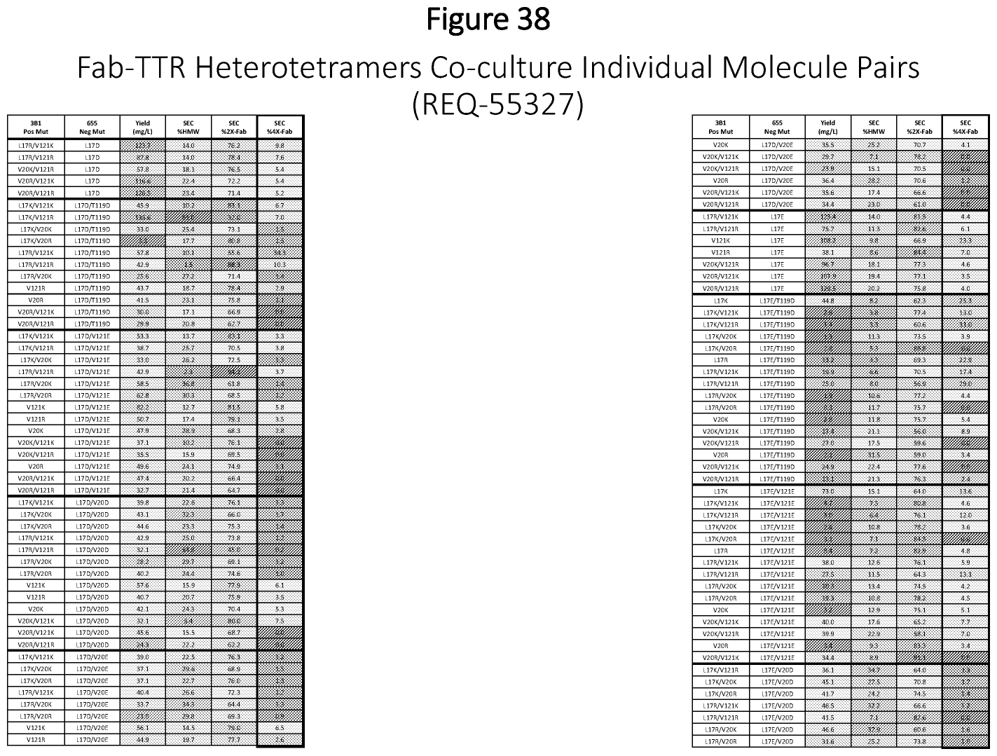

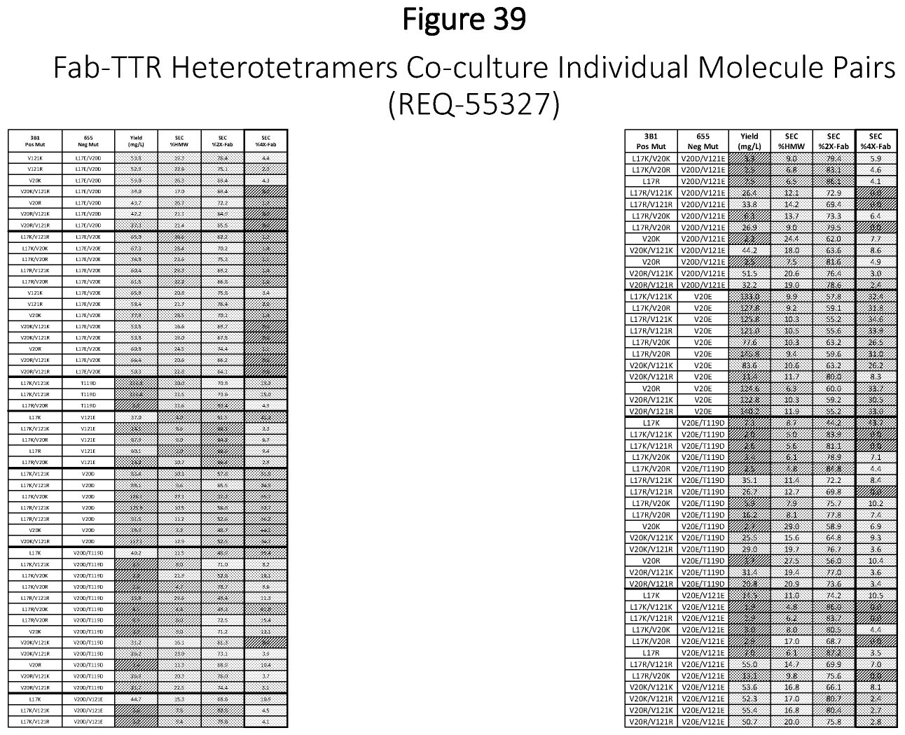

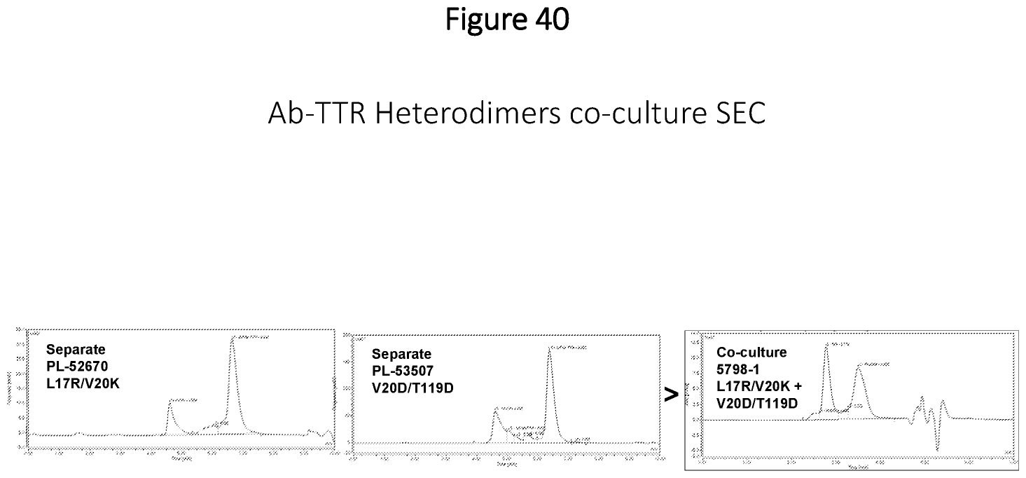

Results of these experiments can be found in FIGS. 31-40. As can be seen in FIGS. 35 and 36, certain combinations of variants produced a noticeable increase in 4×-Fab formation compared to that produced for a simple mixture of the individually produced molecules prior to mixture (determined by taking the average of the pre-mix 4×-Fab levels and subtracting that value from the observed 4×-Fab). FIG. 37 exemplifies this by showing the 4×-Fab SEC peak of the mixtures are much greater than the pre-mix 4×-Fab peaks. In addition, certain co-culture conditions resulted in the formation of higher quantities of 4×-Fab (FIGS. 38 and 39). FIG. 40 shows that the co-cultured 4×-Fab peak is significantly greater than the 4×-Fab peaks of the individually cultured molecules indicating a possible increase in 4×-Fab formation in the presence of the charge counter variants.

SEQ

| ID NO | Description | Sequence |
|---|---|---|
| 1 | Human | GPTGTGESKCPLMVKVLDAVRGSPAINVAVHVFRKAADDTWEPFASGKTSESGELH |
| | TTR AA | GLTTEEEFVEGIYKVEIDTKSYWKALGISPFHEHAEVVETANDSGPRRYTIAALLSPYS |
| | | YSTTAVVTNPKE |
| 2 | Murine | GPAGAGESKCPLMVKVLDAVRGSPAVDVAVKVFKKTSEGSWEPFASGKTAESGELH |
| | TTR AA | GLTTDEKFVEGVYRVELDTKSYWKTLGISPFHEFADVVFTANDSGHRHYTIAALLSP |
| | | YSYSTTAVVSNPQN |
| 3 | Murine | ACAGAAGTCCACTCATTCTTGGCAGGATGGCTTCTCATCGTCTGCTCCTCCTCTGC |
| | TTR NA | CTTGCTGGACTGGTATTTGTGTCTGAGGCTGGCCCTACGGGCACCGGTGAATCCA |
| | | AGTGTCCTCTGATGGTCAAAGTTCTAGATGCTGTCCGAGGCAGTCCTGCCATCAA |
| | | TGTGGCCGTGCATGTGTTCAGAAAGGCTGCTGATGACACCTGGGAGCCATTTGCC |
| | | TCTGGGAAAACCAGTGAGTCTGGAGAGCTGCATGGGCTCACAACTGAGGAGGAA |
| | | TTTGTAGAAGGGATATACAAAGTGGAAATAGACACCAAATCTTACTGGAAGGCA |
| | | CTTGGCATCTCCCCATTCCATGAGCATGCAGAGGTGGTATTCACAGCCAACGACT |
| | | CCGGCCCCCGCCGCTACACCATTGCCGCCCTGCTGAGCCCCTACTCCTATTCCAC |
| | | CACGGCTGTCGTCACCAATCCCAAGGAATGAGGGACTTCTCCTCCAGTGGACCTG |
| | | AAGGACGAGGGATGGGATTTCATGTAACCAAGAGTATTCCATTTTTACTAAAGCA |
| | | CTGTTTTCACCTCATATGCTATGTTAGAAGTCCAGGCAGAGACAATAAAACATTC |
| | | CTGTGAAAGGC |
| 4 | DNP 3B1 | MDMRVPAQLLGLLLLWLRGARCDIQMTQSPSSLSASEGDRVTITCRASQGIRNDLG |
| | Ab LC AA | WYQQKPGKAPKRLIYAASSLQSGVPLRFSGSGSGTEFTLTISSLQPEDFATYYCLQYN |
| | | SYPWTFGQGTKVEIKRTVAAPSVFIFPPSDEQLKSGTASVVCLLNNFsYPREAKVQW |
| | | KVDNALQSGNSQESVTEQDSKDSTYSLSSTLTLSKADYEKHKVYACEVTHQGLSSPV |
| | | TKSFNRGEC |
| 5 | DNP 3B1 | ATGGACATGAGGGTGCCCGCTCAGCTCCTGGGGCTCCTGCTGCTGTGGCTGAGAG |
| | Ab LC NA | GTGCGCGCTGTGACATCCAGATGACCCAGTCTCCATCCTCCCTGTCTGCATCTGA |
| | | AGGAGACAGAGTCACCATCACTTGCCGGGCAAGTCAGGGCATTAGAAATGATT |
| | | TAGGCTGGTATCAGCAGAAACCAGGGAAAGCCCCTAAGCGCCTGATCTATGCTG |
| | | CATCCAGTTTGCAAAGTGGGGTCCCATTAAGGTTCAGCGGCAGTGGATCTGGGA |
| | | CAGAATTCACTCTCACAATCAGCAGCCTGCAGCCTGAAGATTTTGCAACTTATTA |
| | | CTGTCTACAGTATAATAGTTACCCGTGGACGTTCGGCCAAGGGACCAAGGTGG |
| | | AAATCAAACGTACGGTGGCTGCACCATCTGTCTTCATCTTCCCGCCATCTGATGA |
| | | GCAGTTGAAATCTGGAACTGCCTCTGTTGTGTGCCTGCTGAATAACTTCTATCCC |
| | | AGAGAGGCCAAAGTACAGTGGAAGGTGGATAACGCCCTCCAATCGGGTAACTCC |
| | | CAGGAGAGTGTCACAGAGCAGGACAGCAAGGACAGCACCTACAGCCTCAGCAG |
| | | CACCCTGACGCTGAGCAAAGCAGACTACGAGAAACACAAAGTCTACGCCTGCGA |
| | | AGTCACCCATCAGGGCCTGAGCTCGCCCGTCACAAAGAGCTTCAACAGGGGAGA |
| | | GTGT |
| 6 | DNP 3B1 | MDMRVPAQLLGLLLLWLRGARCDIQMTQSPSSLSASEGDRVTITCRASQGIRNDLG |
| | Ab LC AA | WYQQKPGKAPKRLIYAASSLQSGVPLRFSGSGSGTEFTLTISSLQPEDFATYYCLQYN |
| | w/CPM | SYPWTFGQGTKVEIKRTVAAPSVFIFPPSDEQLKSGTASVVCLLNNFYPREAKVQWK |
| | S230E | VDNALQSGNSQESVTEQDSKDSTYSLESTLTLSKADYEKEKVYACEVTHQGLSSPVT |
| | (EU S176E) | KSFNRGEC |
| 7 | DNP 3B1 | ATGGACATGAGGGTGCCCGCTCAGCTCCTGGGGCTCCTGCTGCTGTGGCTGAGAG |
| | Ab LC NA | GTGCGCGCTGTGACATCCAGATGACCCAGTCTCCATCCTCCCTGTCTGCATCTGA |
| | w/CPM | AGGAGACAGAGTCACCATCACTTGCCGGGCAAGTCAGGGCATTAGAAATGATT |
| | S230E | TAGGCTGGTATCAGCAGAAACCAGGGAAAGCCCCTAAGCGCCTGATCTATGCTG |
| | (EU S176E) | CATCCAGTTTGCAAAGTGGGGTCCCATTAAGGTTCAGCGGCAGTGGATCTGGGA |
| | | CAGAATTCACTCTCACAATCAGCAGCCTGCAGCCTGAAGATTTTGCAACTTATTA |
| | | CTGTCTACAGTATAATAGTTACCCGTGGACGTTCGGCCAAGGGACCAAGGTGG |
| | | AAATCAAACGTACGGTGGCTGCACCATCTGTCTTCATCTTCCCGCCATCTGATGA |
| | | GCAGTTGAAATCTGGAACTGCCTCTGTTGTGTGCCTGCTGAATAACTTCTATCCC |
| | | AGAGAGGCCAAAGTACAGTGGAAGGTGGATAACGCCCTCCAATCGGGTAACTCC |
| | | CAGGAGAGTGTCACAGAGCAGGACAGCAAGGACAGCACCTACAGCCTCGAAAG |
| | | CACCCTGACGCTGAGCAAAGCAGACTACGAGAAACACAAAGTCTACGCCTGCGA |
| | | AGTCACCCATCAGGGCCTGAGCTCGCCCGTCACAAAGAGCTTCAACAGGGGAGA |
| | | GTGT |
| 8 | DNP 3B1 | MDMRVPAQLLGLLLLWLRGARCQVQLQESGPGLVKPSETLSLTCTVSGGSISSYYW |
| | Ab HC AA | SWIRQPPGKGLEWIGYIYYSGNTNSNPSLKSRVTISVDTSKNQFSLKLSSVTAADTAV |
| | | YYCARTYYDSSGYYYRAFDIWGQGTMVTVSSASTKGPSVFPLAPSSKSTSGGTAAL |
| | | GCLVKDYFPEPVTVSWNSGALTSGVHTFPAVLQSSGLYSLSSVVTVPSSSLGTQTYIC |
| | | NVNHKPSNTKVDKKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRVVSVLTVLHQD |
| | | WLNGKEYKCKVSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKSRWQQGNVFSCS |
| | | VMHEALHNHYTQKSLSLSPG |
| 9 | DNP 3B1 | ATGGACATGAGGGTGCCCGCTCAGCTCCTGGGGCTCCTGCTGCTGTGGCTGAGAG |
| | Ab HC NA | GTGCGCGCTGTCAGGTGCAGCTGCAGGAGTCGGGCCCAGGACTGGTTAAGCCTT |
| | | CGGAGACCCTGTCCCTCACCTGCACTGTCTCTGGTGGCTCCATCAGTAGTTACTA |
| | | CTGGAGCTGGATCCGGCAGCCCCCAGGGAAGGGACTGGAGTGGATTGGGTATA |
| | | TCTATTACAGTGGGAACACCAACTCCAACCCCTCCCTCAAGAGTCGAGTCAC |
| | | CATATCAGTAGACACGTCCAAGAACCAGTTCTCCCTGAAGCTGAGCTCTGTGACC |
| | | GCTGCGGACACGGCCGTGTATTACTGTGCGAGAACCTACTATGATAGTAGTGG |
| | | TTACTACTACCGTGCTTTTGATATCTGGGGCCAAGGGACAATGGTCACCGTCTC |
| | | TAGTGCCTCCACCAAGGGCCCATCGGTCTTCCCCCTGGCACCCTCCTCCAAGAGC |
| | | ACCTCTGGGGGCACAGCGGCCCTGGGCTGCCTGGTCAAGGACTACTTCCCCGAAC |
| | | CGGTGACGGTGTCGTGGAACTCAGGCGCCCTGACCAGCGGCGTGCACACCTTCCC |
| | | GGCTGTCCTACAGTCCTCAGGACTCTACTCCCTCAGCAGCGTGGTGACCGTGCCC |
| | | TCCAGCAGCTTGGGCACCCAGACCTACATCTGCAACGTGAATCACAAGCCCAGC |
| | | AACACCAAGGTGGACAAGAAAGTTGAGCCCAAATCTTGTGACAAAACTCACACA |
| | | TGCCCACCGTGCCCAGCACCTGAACTCCTGGGGGGACCGTCAGTCTTCCTCTTCC |
| | | CCCCAAAACCCAAGGACACCCTCATGATCTCCCGGACCCCTGAGGTCACATGCGT |
| | | GGTGGTGGACGTGAGCCACGAAGACCCTGAGGTCAAGTTCAACTGGTACGTGGA |
| | | CGGCGTGGAGGTGCATAATGCCAAGACAAAGCCGCGGGAGGAGCAGTACAACA |
| | | GCACGTACCGTGTGGTCAGCGTCCTCACCGTCCTGCACCAGGACTGGCTGAATGG |
| | | CAAGGAGTACAAGTGCAAGGTCTCCAACAAAGCCCTCCCAGCCCCCATCGAGAA |
| | | AACCATCTCCAAAGCCAAAGGGCAGCCCCGAGAACCACAGGTGTACACCCTGCC |
| | | CCCATCCCGGGAGGAGATGACCAAGAACCAGGTCAGCCTGACCTGCCTGGTCAA |
| | | AGGCTTCTATCCCAGCGACATCGCCGTGGAGTGGGAGAGCAATGGGCAGCCGGA |
| | | GAACAACTACAAGACCACGCCTCCCGTGCTGGACTCCGACGGCTCCTTCTTCCTC |
| | | TATAGCAAGCTCACCGTGGACAAGAGCAGGTGGCAGCAGGGGAACGTCTTCTCA |
| | | TGCTCCGTGATGCATGAGGCTCTGCACAACCACTACACGCAGAAGAGCCTCTCCC |
| | | TGTCTCCGGGT |
| 10 | DNP 3B1 | MDMRVPAQLLGLLLLWLRGARCQVQLQESGPGLVKPSETLSLTCTVSGGSISSYYYW |
| | Ab HC AA | SWIRQPPGKGLEWIGYIYYSGNTNSNPSLKSRVTISVDTSKNQFSLKLSSVTAADTAV |
| | w/CPM | YYCARTYYDSSGYYYRAFDIWGQGTMVTVSSASTKGPSVFPLAPSSKSTSGGTAAL |
| | S230K | GCLVDKYFPEPVTVSWNSGALTSGVHTFPAVLQSSGLYSLKSVVTVPSSSLGTQTYIC |
| | (EU S183K) | NVNHKPSNTKVDKKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRVVSVLTVLHQD |
| | | WLNGKEYKCKVSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKSRWQQGNVFSCS |
| | | VMHEALHNHYTQKSLSLSPG |
| 11 | DNP 3B1 | ATGGACATGAGGGTGCCCGCTCAGCTCCTGGGGCTCCTGCTGCTGTGGCTGAGAG |
| | Ab HC NA | GTGCGCGCTGTCAGGTGCAGCTGCAGGAGTCGGGCCCAGGACTGGTTAAGCCTT |
| | w/CPM | CGGAGACCCTGTCCCTCACCTGCACTGTCTCTGGTGGCTCCATCAGTAGTTACTA |
| | S230K | CTGGAGCTGGATCCGGCAGCCCCCAGGGAAGGGACTGGAGTGGATTGGGTATA |
| | (EU S183K) | TCTATTACAGTGGGAACACCAACTCCAACCCCTCCCTCAAGAGTCGAGTCAC |
| | | CATATCAGTAGACACGTCCAAGAACCAGTTCTCCCTGAAGCTGAGCTCTGTGACC |
| | | GCTGCGGACACGGCCGTGTATTACTGTGCGAGAACCTACTATGATAGTAGTGG |
| | | TTACTACTATCCGTGCTTTTGATATCTGGGGCCAAGGGACAATGGTCACCGTCTC |
| | | TAGTGCCTCCACCAAGGGCCCATCGGTCTTCCCCCTGGCACCCTCCTCCAAGAGC |
| | | ACCTCTGGGGGCACAGCGGCCCTGGGCTGCCTGGTCAAGGACTACTTCCCCGAAC |
| | | CGGTGACGGTGTCGTGGAACTCAGGCGCCCTGACCAGCGGCGTGCACACCTTCCC |
| | | GGCTGTCCTACAGTCCTCAGGACTCTACTCCCCAAGAGCGTGGTGACCGTGCCC |
| | | TCCAGCAGCTTGGGCACCCAGACCTACATCTGCAACGTGAATCACAAGCCCAGC |
| | | AACACCAAGGTGGACAAGAAAGTTGAGCCCAAATCTTGTGACAAAACTCACACA |
| | | TGCCCACCGTGCCCAGGACCTGAACTCCTGGGGGGACCGTCAGTCTTCCTCTTCC |
| | | CCCCAAAACCCAAGGACACCCTCATGATCTCCCGGACCCCTGAGGTCACATGCGT |
| | | GGTGGTGGACGTGAGCCACGAAGACCCTGAGGTCAAGTTCAACTGGTACGTGGA |
| | | CGGCGTGGAGGTGCATAATGCCAAGACAAAGCCGCGGGAGGAGCAGTACAACA |
| | | GCACGTACCGTGTGGTCAGCGTCCTCACCGTCCTGCACCAGGACTGGCTGAATGG |
| | | CAAGGAGTACAAGTGCAAGGTCTCCAACAAAGCCGCCCAGCCCCCATCGAGAA |
| | | AACCATCTCCAAAGCCAAAGGGCAGCCCCGAGAACCACAGGTGTACACCCTGCC |
| | | CCCATCCCGGGAGGAGATGACCAAGAACCAGGTCAGCCTGACCTGCCTGGTCAA |
| | | AGGCTTCTATCCCAGCGACATCGCCGTGGAGTGGGAGAGCAATGGGCAGCCGGA |
| | | GAACAACTACAAGACCACGCCTCCCGTGCTGGACTCCGACGGCTCCTTCTTCCTC |
| | | TATAGCAAGCTCACCGTGGACAAGAGCAGGTGGCAGCAGGGGAACGTCTTCTCA |
| | | TGCTCCGTGATGCATGAGGCTCTGCACAACCACTACACGCAGAAGAGCCTCTCCC |
| | | TGTCTCCGGGT |
| 12 | DNP 3B1 | MDMRVPAQLLGLLLLWLRGARCQVQLQESGPGLVKPSETLSLTCTVSGGSISSYYW |
| | Fab HC AA | SWIRQPPGKGLEWIGYIYYSGNTNSNPSLKSRVTISVDTSKNQFSLKLSSVTAADTAV |
| | | YYCARTYYDSSGYYYRAFDIWGQGTMVTVSSASTKGPSVFPLAPSSKSTSGGTAAL |
| | | GCLVKDYFPEPVTVSWNSGALTSGVHTFPAVLQSSGLYSLSSVVTVPSSSLGTQTYIC |
| | | NVNHKPSNTKVDKKVEPKSC |
| 13 | DNP 3B1 | ATGGACATGAGGGTGCCCGCTCAGCTCCTGGGGCTCCTGCTGCTGTGGCTGAGAG |
| | Fab HC AA | GTGCGCGCTGTCAGGTGCAGCTTGCAGGAGTCGGGCCCAGGACTGGTTAAGCCTT |
| | | CGGAGACCCTGTCCCTCACCTGCACTGTCTCTGGTGGCTCCATCAGTAGTTACTA |
| | | CTGGAGCTGGATCCGGCAGCCCCCAGGGAAGGGACTGGAGTGGATTGGGTATA |
| | | TCTATTACAGTGGGAACACCAACTCCAACCCCTCCCTCAAGAGTCGAGTCAC |
| | | CATATCAGTAGACACGTCCAAGAACCAGTTCTCCCTGAAGCTGAGCTCTGTGACC |
| | | GCTGCGGACACGGCCGTGTATTACTGTGCGAGAACCTACTATGATAGTAGTGG |
| | | TTACTACTACCGTGCTTTTGATATCTGGGGCCAAGGGACAATGGTCACCGTCTC |
| | | TAGTGCCTCCACCAAGGGCCCATCGGTCTTCCCCCTGGCACCCTCCTCCAAGAGC |
| | | ACCTCTGGGGGCACAGCGGCCCTGGGCTGCCTGGTCAAGGACTACTTCCCCGAAC |
| | | CGGTGACGGTGTCGTGGAACTCAGGCGCCCTGACCAGCGGCGTGCACACCTTCCC |
| | | GGCTGTCCTACAGTCCTCAGGACTCTACTCCCTCAGCAGCGTGGTGACCGTGCCC |
| | | TCCAGCAGCTTGGGCACCCAGACCTACATCTGCAACGTGAATCACAAGCCCAGC |
| | | AACACCAAGGTGGACAAGAAAGTTGAGCCCAAATCTTGT |
| 14 | DNP 3B1 | MDMRVPAQLLGLLLLWLRGARCQVQLQESGPGLVKPSETLSLTCTVSGGSISSYYW |
| | Fab HC AA | SWIRQPPGKGLEWIGYIYYSGNTNSNPSLKSRVTISVDTSKNQFSLKESSVTAADTAV |
| | w/CPM | YYCARTYYDSSGYYYRAFDIWGQGTMVTVSSASTKGPSVFPLAPSSKSTSGGTAAL |
| | S230K | GCLVKDYFPEPVTVSWNSGALTSGVHTFPAVLQSSGLYSLKSVVTVPSSSLGTQTYIC |
| | (EU S183K) | NVNHKPSNTKVDKKVEPKSC |
| 15 | DNP 3B1 | ATGGACATGAGGGTGCCCGCTCAGCTCCTGGGGCTCCTGCTGCTGTGGCTGAGAG |
| | Fab HC NA | GTGCGCGCTGTCAGGTGCAGCTGCAGGAGTCGGGCCCAGGACTGGTTAAGCCTT |
| | | CGGAGACCCTGTCCCTCACCTGCACTGTCTCTGGTGGCTCCATCAGTAGTTACTA |
| | | CTGGAGCTGGATCCGGCAGCCCCCAGGGAAGGGACTGGAGTGGATTGGGTATA |
| | | TCTATTACAGTGGGAACACCAACTCCAACCCCTCCCTCAAGAGTCGAGTCAC |
| | | CATATCAGTAGACACGTCCAAGAACCAGTTCTCCCTGAAGCTGAGCTCTGTGACC |
| | | GCTGCGGACACGGCCGTGTATTACTGTGCGAGAACCTACTATGATAGTAGTGG |
| | | TTACTACTACCGTGCTTTTGATATCTGGGGCCAAGGGACAATGGTCACCGTCTC |
| | | TAGTGCCTCCACCAAGGGCCCATCGGTCTTCCCCCTGGCACCCTCCTCCAAGAGC |
| | | ACCTCTGGGGGCACAGCGGCCCTGGGCTGCCTGGTCAAGGACTACTTCCCCGAAC |
| | | CGGTGACGGTGTCGTGGAACTCAGGCGCCCTGACCAGCGGCGTGCACACCTTCCC |
| | | GGCTGTCCTACAGTCCTCAGGACTCTACTCCCTCAAGAGCGTGGTGACCGTGCCC |
| | | TCCAGCAGCTTGGGCACCCAGACCTACATCTGCAACGTGAATCACAAGCCCAGC |
| | | AACACCAAGGTGGACAAGAAAGTTGAGCCCAAATCTTGT |
| 16 | 655-341 Ab | METPAQLLFLLLLWLPDTTGEIVLTQSPGTESLSPGERATLSCRASQGISRSELAWYQ |
| | LC AA | QKPGQAPSLLIYGASSRATGIPDRFSGSGSGTDFTLTISRLEPEDFAVYYCQQFGSSP |
| | | WTFGQGTKVEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGLSSPVTKS |
| | | FNRGEC |
| 17 | 655-341 Ab | ATGGAAACCCCAGCGCAGCTTCTCTTCCTCCTGCTACTCTGGCTCCCAGATACCA |
| | LC NA | CCGGAGAAATTGTGTTGACGCAGTCTCCAGGCACCCTGTCTTTGTCTCCAGGGGA |
| | | AAGAGCCACCCTCTCCTGCAGGGCCAGTCAGGGTATTAGTAGAAGCGAATTA |
| | | GCCTGGTACCAGCAGAAACCTGGCCAGGCTCCCAGCCTCCTCATCTATGGTGCA |
| | | TCCAGCAGGGCCACTGGCATCCCAGACAGGTTCAGTGGCAGTGGGTCTGGGAC |
| | | AGACTTCACTCTCACCATCAGCAGACTGGAGCCTGAAGATTTTGCAGTGTATTAC |
| | | TGTCAACAATTTGGTAGTTCACCGTGGACGTTCGGCCAAGGGACCAAGGTGGA |
| | | AATCAAACGAACTGTGGCTGCACCATCTGTCTTCATCTTCCCGCCATCTGATGAG |
| | | CAGTTGAAATCTGGAACTGCTAGCGTTGTGTGCCTGCTGAATAACTTCTATCCCA |
| | | GAGAGGCCAAAGTACAGTGGAAGGTGGATAACGCCCTCCAATCGGGTAACTCCC |
| | | AGGAGAGTGTCACAGAGCAGGACAGCAAGGACAGCACCTACAGCCTCAGCAGC |
| | | ACCCTGACGCTGAGCAAAGCAGACTACGAGAAACACAAAGTCTACGCCTGCGAA |
| | | GTCACCCATCAGGGCCTGAGCTCGCCCGTCACAAAGAGCTTCAACAGGGGAGAG |
| | | TGT |
| 18 | 655-341 Ab | METPAQLLFLLLLWLPDTTGEIVLTQSPGTLSLSPGERATLSCRASQGISRSELAWYQ |
| | LC AA | QKPGQAPSLLIYGASSRATGIPDRFSGSGSGTDFTLTISRLEPEDFAVYYCQQFGSSP |
| | w/CPM | WTFGQGTKVEIKRTVAAPSVFIFPPSDEQLKSGTAVVCLLNNFYPREAKVQWKVD |
| | S230K | NALQSGNSQESVTEQDSKDSTYSLKSTLTLSKADYEKHKVYACEVTHQGLSSPVTKS |
| | (EU S176K) | FNRGEC |
| 19 | 655-341 Ab | ATGCAAACCCCAOCGCAGCTTCTCTTCCTCCTGCTACTCTGGCTCCCAGATACCA |
| | LC NA | CCGGAGAAATTGTGTTGACGCAGTCTCCAGGCACCCTGTCTTTGTCTCCAGGGGA |
| | w/CPM | AAGAGCCACCCTCTCCTGCAGGGCCAGTCAGGGTATTAGTAGAAGCGAATTA |
| | S230K | GCCTGGTACCAGCAGAAACCTGGCCAGGCTCCCAGCCTCCTCATCTATGGTGCA |
| | (EU S176K) | TCCAGCAGGGCCACTGGCATCCCAGACAGGTTCAGTGGCAGTGGGTCTGGGAC |
| | | AGACTTCACTCTCACCATCAGCAGACTGGAGCCTGAAGATTTTGCAGTGTATTAC |
| | | TGTCAACAATTTGGTAGTTCACCGTGGACGTTCGGCCAAGGGACCAAGGTGGA |
| | | AATCAAACGAACTGTGGCTGCACCATCTGTCTTCATCTTCCCGCCATCTGATGAG |
| | | CAGTTGAAATCTGGAACTGCTAGCGTTGTGTGCCTGCTGAATAACTTCTATCCCA |
| | | GAGAGGCCAAAGTACAGTGGAAGGTGGATAACGCCCTCCAATCGGGTAACTCCC |
| | | AGGAGAGTGTCACAGAGCAGGACAGCAAGGACAGCACCTACAGCCTCAAGAGC |
| | | ACCCTGACGCTGAGCAAAGCAGACTACGAGAAACACAAAGTCTACGCCTGCGAA |
| | | GTCACCCATCAGGGCCTGAGCTCGCCCGTCACAAAGAGCTTCAACAGGGGAGAG |
| | | TGT |
| 20 | 655-341 Ab | MKHLWFFLLLVAAPRWVLSQVQLQESGPGLVKPSQTLSLTCTVSGGSISSGDYFWS |
| | HC AA | WIRQLPGKGLEWIGHIHNSGTTYYNPSLKSRVTISVDTSKKQFSLRLSSVTAADTAV |
| | | YYCARDRGGDYAYGMDVWGQGTTVTVSSASTKGPSVFPLAPSSKSTSGGTAALGC |
| | | LVKDYFPEPVTVSWNSGALTSGVHTFPAVLQSSGLYSLSSVVTVPSSSLGTQTYICNV |
| | | NHKPSNTKVDKRVEPKSCDKTHTCPPCPAPELLGGPSVFLFPPRPKDTLMISRTPEVT |
| | | CVVVDVSHEDPEVKFNWYVDGVEVHNAKTKPREEQYNSTYRVVSVLTVLHQDWL |
| | | NGKEYKCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVFSCSVM |
| | | HEALHNHYTQKSLSLSPG |
| 21 | 655-341 Ab | ATGAAGCACCTGTGGTTCTTCCTCCTGCTGGTGGCAGCTCCCAGATGGGTCCTGT |
| | HC NA | CCCAGGTGCAGCTGCAGGAGTCGGGCCCAGGACTGGTGAAGCCTTCACAGACCC |
| | | TGTCCCTCACCTGCACTGTCTCTGGTGGCTCCATCAGCAGTGGTGATTACTTCTG |
| | | GAGCTGGATCCGCCAGCTCCCAGGGAAGGGCCTGGAGTGGATTGGGCACATCC |
| | | ATAACAGTGGGACCACCTACTACAATCCGTCCCTCAAGAGTCGAGTTACCAT |
| | | ATCAGTAGACACGTCTAAGAAGCAGTTCTCCCTGAGGCTGAGTTCTGTGACTGCC |
| | | GCGGACACGGCCGTATATTACTGTGCGAGAGATCGAGGGGGTGACTACGCTTA |
| | | TGGTATGGACGTCTGGGGCCAAGGGACCACGGTCACCGTCTCCTCAGCCTCCAC |
| | | CAAGGGCCCATCCGTCTTCCCCCTGGCACCCTCCTCCAAGAGCACCTCTGGGGGC |
| | | ACAGCGGCCCTGGGCTGCCTGGTCAAGGACTACTTCCCCGAACCGGTGACGGTGT |
| | | CGTGGAACTCAGGCGCCCTGACCAGCGGCGTGCACACCTTCCCGGCTGTCCTACA |
| | | GTCCTCAGGACTCTACTCCCTCAGCAGCGTGGTGACCGTGCCCTCCAGCAGCTTG |
| | | GGCACCCAGACCTACATCTGCAACGTGAATCACAAGCCCAGCAACACCAAGGTG |
| | | GACAAGAGAGTTGAGCCCAAATCTTGTGACAAAACTCACACATGCCCACCGTGC |
| | | CCAGCACCTGAACTCCTGGGGGGACCGTCAGTCTTCCTCTTCCCCCCAAAACCCA |
| | | AGGACACCCTCATGATCTCCCGGACCCCTGAGGTCACATGCGTGGTGGTGGACGT |
| | | GAGCCACGAAGACCCTGAGGTCAAGTTCAACTGGTACGTGGACGGCGTGGAGGT |
| | | GCATAATGCCAAGACAAAGCCGCGGGAGGAGCAGTACAACAGCACGTACCGTGT |
| | | GGTCAGCGTCCTCACCGTCCTGCACCAGGACTGGCTGAATGGCAAGGAGTACAA |
| | | GTGCAAGGTCTCCAACAAAGCCCTCCCAGCCCCCATCGAGAAAACCATCTCCAA |
| | | AGCCAAAGGGCAGCCCCGAGAACCACAGGTGTACACCCTGCCCCCATCCCGGGA |
| | | GGAGATGACCAAGAACCAGGTCAGCCTGACCTGCCTGGTCAAAGGCTTCTATCC |
| | | CAGCGACATCGCCGTGGAGTGGGAGAGCAATGGGCAGCCGGAGAACAACTACA |
| | | AGACCACGCCTCCCGTGCTGGACTCCGACGGCTCCTTCTTCCTCTATAGCAAGCT |
| | | CACCGTGGACAAGAGCAGGTGGCAGCAGGGGAACGTCTTCTCATGCTCCGTGAT |
| | | GCATGAGGCTCTGCACAACCACTACACGCAGAAGAGCCTCTCCCTGTCTCCGGGT |
| 22 | 655-341 Ab | MKHLWFFLLLVAAPRWVLSQVQLQESGPGLVKPSQTLSLTCTVSGGSISSGDYFWS |
| | HC AA | WIRQLPGKGLEWIGHIHNSGTTYYNPSLKSRVTISVDTSKKQFSLRLSSVTAADTAV |
| | w/CPM | YYCARDRGGDYAYGMDVWGQGTTVTVSSASTKGPSVFPLAPSSKSTSGGTAALGC |
| | S230E | LVKDYFPEPVTVSWMSGALTSGVHTFPAVLQSSGLYSLESVVTVPSSSLGTQTYICNV |
| | (EU S183E) | NHKPSNTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLTVLHQDWL |
| | | NGKEYKCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVFSCSVM |
| | | HEALHNHYTQKSLSLSPG |
| 23 | 655-341 Ab | ATGAAGCACCTGTGGTTCTTCCTCCTGCTGGTGGCAGCTCCCAGATGGGTCCTGT |
| | HC NA | CCCAGGTGCAGCTGCAGGAGTCGGGCCCAGGACTGGTGAAGCCTTCACAGACCC |
| | w/CPM | TGTCCCTCACCTGCACTGTCTCTGGTGGCTCCATCAGCAGTGGTGATTACTTCTG |
| | S230E | GAGCTGGATCCGCCAGCTCCCAGGGAAGGGCCTGGAGTGGATTGGGCACATCC |
| | (EU S183E) | ATAACAGTGGGACCACCTACTACAATCCGTCCCTCAAGAGTCGAGTTACCAT |
| | | ATCAGTAGACACGTCTAAGAAGCAGTTCTCCCTGAGGCTGAGTTCTGTGACTGCC |
| | | GCGGACACGGCCGTATATTACTGTGCGAGAGATCGAGGGGGTGACTACGCTTA |
| | | TGGTATGGACGTCTGGGGCCAAGGGACCACGGTCACCGTCTCCTCAGCCTCCAC |
| | | CAAGGGCCCATCCGTCTTCCCCCTGGCACCCTCCTCCAAGAGCACCTCTGGGGGC |
| | | ACAGCGGCCCTGGGCTGCCTGGTCAAGGACTACTTCCCCGAACCGGTGACGGTGT |
| | | CGTGGAACTCAGGCGCCCTGACCAGCGGCGTGCACACCTTCCCGGCTGTCCTACA |
| | | GTCCTCAGGACTCTACTCCCTCGAGAGCGTGGTGACCGTGCCCTCCAGCAGCTTG |
| | | GGCACCCAGACCTACATCTGCAACGTGAATCACAAGCCCAGCAACACCAAGGTG |
| | | GACAAGAGAGTTGAGCCCAAATCTTGTGACAAAACTCACACATGCCCACCGTGC |
| | | CCAGCACCTGAACTCCTGGGGGGACCGTCAGTCTTCCTCTTCCCCCCAAAACCCA |
| | | AGGACACCCTCATGATCTCCCGGACCCCTGACTGTCACATGCGTGGTGGTGGACGT |
| | | GAGCCACGAAGACCCTGAGGTCAAGTTCAACTGGTACGTGGACGGCGTGGAGGT |
| | | GCATAATGCCAAGACAAAGCCGCGGGAGGAGCAGTACAACAGCACGTACCGTGT |
| | | GGTCAGCGTCCTCACCGTCCTGCACCAGGACTGGCTGAATGGCAAGGAGTACAA |
| | | GTGCAAGGTCTCCAACAAAGCCCTCCCAGCCCCCATCGAGAAAACCATCTCCAA |
| | | AGCCAAAGGGCAGCCCCGAGAACCACAGGTGTACACCCTGCCCCCATCCCGGGA |
| | | GGAGATGACCAAGAACCAGGTCAGCCTGACCTGCCTGGTCAAAGGCTTCTATCC |
| | | CAGCGACATCGCCGTGGAGTGGGAGAGCAATGGGCAGCCGGAGAACAACTACA |
| | | AGACCACGCCTCCCGTGCTGGACTCCGACGGCTCCTTCTTCCTCTATAGCAAGCT |
| | | CACCGTGGACAAGAGCAGGTGGCAGCAGGGGAACGTCTTCTCATGCTCCGTGAT |
| | | GCATGAGGCTCTGCACAACCACTACACGCAGAAGAGCCTCTCCCTGTCTCCGGGT |
| 24 | 655-341 | MKHLWFFLLLVAAPRWVLSQVQLQESGPGLVKPSQTLSLTCTVSGGSISSGDVFWS |
| | Fab HC AA | WIRQLPGKGLEWIGHIHNSGTTYYNPSLKSRVTISVDTSKKQFSLRLSSVTAADTAV |
| | | YYCARDRGGDYAYGMDVWGQGTTVTVSSASTKGPSVFPLAPSSKSTSGGTAALGC |
| | | LVKDYFPEPVTVSWNSGALTSGVHTFPAVLQSSGLYSLSSVVTVPSSSLGTQTYICNV |
| | | NHKPSNTKVDKRVEPKSC |
| 25 | 655-341 | ATGAAGCACCTGTGGTTCTTCCTCCTGCTGGTGGCAGCTCCCAGATGGGTCCTGT |
| | Fab HC NA | CCCAGGTGCAGCTGCAGGAGTCGGGCCCAGGACTGGTGAAGCCTTCACAGACCC |
| | | TGTCCCTCACCTGCACTGTCTCTGGTGGCTCCATCAGCAGTGGTGATTACTTCTG |
| | | GAGCTGGATCCGCCAGCTCCCAGGGAAGGGCCTGGAGTGGATTGGGCACATCC |
| | | ATAACAGTGGGACCACCTACTACAATCCGTCCCTCAAGAGTCGAGTTACCAT |
| | | ATCAGTAGACACGTCTAAGAAGCAGTTCTCCCTGAGGCTGAGTTCTGTGACTGCC |
| | | GCGGACACGGCCGTATATTACTGTGCGAGAGATCGAGGGGGTGACTACGCTTA |
| | | TGGTATGGACGTCTGGGGCCAAGGGACCACGGTCACCGTCTCCTCAGCCTCCAC |
| | | CAAGGGCCCATCCGTCTTCCCCCTGGCACCCTCCTCCAAGAGCACCTCTGGGGGC |
| | | ACAGCGGCCCTGGGCTGCCTGGTCAAGGACTACTTCCCCGAACCGGTGACGGTGT |
| | | CGTGGAACTCAGGCGCCCTGACCAGCGGCGTGCACACCTTCCCGGCTGTCCTACA |
| | | GTCCTCAGGACTCTACTCCCTCAGCAGCGTGGTGACCGTGCCCTCCAGCAGCTTG |
| | | GGCACCCAGACCTACATCTGCAACGTGAATCACAAGCCCAGCAACACCAAGGTG |
| | | GACAAGAGAGTTGAGCCCAAATCTTGT |
| 26 | 655-341 | MKHLWFFLLLVAAPRWVLSQVQLQESGPGLVKPSQTLSLTCTVSGGSISSGDYFWS |
| | Fab HC AA | WIRQLPGKGLEWIGHIHNSGTTYYNPSLKSRVTISVDTSKKQFSLRLSSVTAADTAV |
| | w/CPM | YYCARDRGGDYAYGMDVWGQGTTVTVSSASTKGPSVFPLAPSSKSTSGGTAALGC |
| | S230E | LVKDYFPEPVTVSWNSGALTSGVHTFPAVLQSSGLYSLESVVTVPSSSLGTQTYICNV |
| | (EU S183E) | NHKPSNTKVDKRVEPKSC |
| 27 | 655-341 | ATGAAGCACCTGTGGTTCTTCCTCCTGCTGGTGGCAGCTCCCAGATGGGTCCTGT |
| | Fab HC NA | CCCAGGTGCAGCTGCAGGAGTCGGGCCCAGGACTGGTGAAGCCTTCACAGACCC |
| | w/CPM | TGTCCCTCACCTGCACTGTCTCTGGTGGCTCCATCAGCAGTGGTGATTACTTCTG |
| | S230E | GAGCTGGATCCGCCAGCTCCCAGGGAAGGGCCTGGAGTGGATTGGGCACATCC |
| | (EU S183E) | ATAACAGTGGGACCACCTACTACAATCCGTCCCTCAAGAGTCGAGTTACCAT |
| | | ATCAGTAGACACGTCTAAGAAGCAGTTCTCCCTGAGGCTGAGTTCTGTGACTGCC |
| | | GCGGACACGGCCGTATATTACTGTGCGAGAGATCGAGGGGGTGACTACGCTTA |
| | | TGGTATGGACGTCTGGGGCCAAGGGACCACGGTCACCGTCTCCTCAGCCTCCAC |
| | | CAAGGGCCCATCCGTCTTCCCCCTGGCACCCTCCTCCAAGAGCACCTCTGGGGGC |
| | | ACAGCGGCCCTGGGCTGCCTGGTCAAGGACTACTTCCCCGAACCGGTGACGGTGT |
| | | CGTGGAACTCAGGCGCCCTGACCAGCGGCGTGCACACCTTCCCGGCTGTCCTACA |
| | | GTCCTCAGGACTCTACTCCCTCGAGAGCGTGGTGACCGTGCCCTCCAGCAGCTTG |
| | | GGCACCCAGACCTACATCTGCAACGTGAATCACAAGCCCAGCAACACCAAGGTG |
| | | GACAAGAGAGTTGAGCCCAAATCTTGT |
| 28 | DNP Ab LC | ATGGACATGAGGGTGCCCGCTCAGCTCCTGGGGCTCCTGCTGCTGTGGCTGAGAG |
| | NA (TTR | GTGCGCGCTGTGACATCCAGATGACCCAGTCTCCATCCTCCCTGTCTGCATCTGA |
| | construct) | AGGAGACAGAGTCACCATCACTTGCCGGGCAAGTCAGGGCATTAGAAATGATT |
| | | TAGGCTGGTATCAGCAGAAACCAGGGAAAGCCCCTAAGCGCCTGATCTATGCTG |
| | | CATCCAGTTTGCAAAGTGGGGTCCCATTAAGGTTCAGCGGCAGTGGATCTGGGA |
| | | CAGAATTCACTCTCACAATCAGCAGCCTGCAGCCTGAAGATTTTGCAACTTATTA |
| | | CTGTCTACAGTATAATAGTTACCCGTGGACGTTCGGCCAAGGGACGAAGGTGG |
| | | AAATCAAACGTACGGTGGCTGCACCATCTGTCTTCATCTTCCCGCCATCTGATGA |
| | | GCAGTTGAAATCTGGAACTGCCTCTGTTGTGTGCCTGCTGAATAACTrCTATCCC |
| | | AGAGAGGCCAAAGTACAGTGGAAGGTGGATAACGGCCTCCAATCGGGTAACTCC |
| | | CAGGAGAGTGTCACAGAGCAGGACAGCAAGGACAGCACCTACAGCCTCAGCAG |
| | | CACCCTGACGCTGAGCAAAGCAGACTACGAGAAACACAAAGTCTACGCCTGCGA |
| | | AGTCACCCATCAGGGCCTGAGCTCGCCCGTCACAAAGAGCTTCAACAGGGGAGA |
| | | GTGT |
| 29 | DNP Ab LC | MDMRVPAQLLGLLLLWLRGARCDIQMTQSPSSLSASEGDRVTITCRASQGIRNDLG |
| | AA (TTR | WYQQKPGKAPKRLIYAASSLQSGVPLRFSGSGSGTEFTLTISSLQPEDFATYYCLQYN |
| | construct) | SYPWTFGQGTKVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTHQGLSSPVT |
| | | KSFNRGEC |
| 30 | DNP Ab | ATGGACATGAGGGTGCCCGCTCAGCTCCTGGGGCTCCTGCTGCTGTGGCTGAGAG |
| | HC NA | GTGCGCGCTGTCAGGTGCAGCTGCAGGAGTCGGGCCCAGGACTGGTTAAGCCTT |
| | (TTR | CGGAGACCCTGTCCCTCACCTGCACTGTCTCTGGTGGCTCCATCAGTAGTTACTA |
| | construct) | CTGGAGCTGGATCCGGCAGCCCCCAGGGAAGGGACTGGAGTGGATTGGGTATA |
| | | TCTATTACAGTGGGAACACCAACTCCAACCCCTCCCTCAAGAGTCGAGTCAC |
| | | CATATCAGTAGACACGTCCAAGAACCAGTTCTCCCTGAAGCTGAGCTCTGTGACC |
| | | GCTGCGGACACGGCCGTGTATTACTGTGCGAGAACCTACTATGATAGTAGTGG |
| | | TTACTACTACCGTGCTTTTGATATCTGGGGCCAAGGGACAATGGTCACCGTCTC |
| | | TAGTGCCTCCACCAAGGGCCCATCGGTCTTCCCCCTGGCACCCTCCTCCAAGAGC |
| | | ACCTCTGGGGGCACAGCGGCCCTGGGCTGCCTGGTCAAGGACTACTTCCCCGAAC |
| | | CGGTGACGGTGTCGTGGAACTCAGGCGCCCTGACCAGCGGCGTGCACACCTTCCC |
| | | GGCTGTCCTACAGTCCTCAGGACTCTACTCCCTCAGCAGCGTGGTGACCGTGCCC |
| | | TCCAGCAGCTTGGGCACCCAGACCTACATCTGCAACGTGAATCACAAGCCCAGC |
| | | AACACCAAGGTGGACAAGAAAGTTGAGCCCAAATCTTGTGACAAAACTCACACA |
| | | TGCCCACCGTGCCCAGCACCTGAACTCCTGGGGGGACCGTCAGTCTTCCTCTTCC |
| | | CCCCAAAACCCAAGGACACCCTCATGATCTCCCGGACCCCTGAGGTCACATGCGT |
| | | GGTGGTGGACGTGAGCCACGAAGACCCTGAGGTCAAGTTCAACTGGTACGTGGA |
| | | CGGCGTGGAGGTGCATAATGCCAAGACAAAGCGGCGGGAGGAGCAGTACAACA |
| | | GCACGTACCGTGTGGTCAGCGTCCTCACCGTCCTGCACCAGGACTGGCTGAATGG |
| | | CAAGGAGTACAAGTGCAAGGTCTCCAACAAAGCCCTCCCAGCCCCCATCGAGAA |
| | | AACCATCTCCAAAGCCAAAGGGCAGCCCCGAGAACCACAGGTGTACACCCTGCC |
| | | CCCATCCCGGGAGGAGATGACCAAGAACCAGGTCAGCCTGACCTGCCTGGTCAA |
| | | AGGCTTCTATCCCAGCGACATCGCCGTGGAGTGGGAGAGCAATGGGCAGCCGGA |
| | | GAACAACTACAAGACCACGCCTCCCGTGCTGGACTCCGACGGCTCCTTCTTCCTC |
| | | TATAGCAAGCTCACCGTGGACAAGAGCAGGTGGCAGCAGGGGAACGTCTTCTCA |
| | | TGCTCCGTGATGCATGAGGCTCTGCACAACCACTACACGCAGAAGAGCCTCTCCC |
| | | TGTCTCCGGGTGGAGGCGGTCCAACTGGTACTGGTGAATCTAAAGCTCCTCTTAT |
| | | GGTTGCAGTCAAAGATGCTGTTCGTGGTTCCCCGGCAATTAATGTTGCTGTACAT |
| | | GTTTTCCGTAAAGCTGCTGACGACACCTGGGAACCGTTCGCTAGCGGTAAAACCT |
| | | CCGAATCCGGTGAACTGCACGGTCTGACCACCGAAGAAGAATTCGTTGAAGGTA |
| | | TCTACAAAGTTGAAATCGACACCAAATCCTACTGGAAAGCTTTGGGTATCTCCCC |
| | | GTTCCACGAACACGCTGAAGTTGTTTTCACCGCTAACGACTCCGGTCCGCGTCGT |
| | | TACACGATCGCTGCTCTGCTGTCCCCGTACTCCTACTCCACCACCGCTGTTGTTAC |
| | | CAACCCGAAAGAA |
| 31 | DNP 3B1 | MDMRVPAQLLGLLLLWLRGARCQVQLQESGPGLVKPSETLSLTCTVSGGSISSYYW |
| | Ab LC AA | SWIRQPPGKGLEWIGYIYYSGNTNSNPSLKSRVTISVDTSKNQPSLKLSSVTAADTAV |
| | (TTR | YYCARTYYDSSGYYYRAFDIWGQGTMVTVSSASTKGPSVFPLAPSSKSTSGGTAAL |
| | construct) | GCLVKDYFPEPVTVSWNSGALTSGVHTFPAVLQSSGLYSLSSVVTVPSSSLGTQTYIC |
| | | NVNHKPSSNTKVDKKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRVVSVLYVLHQD |
| | | WLNGKEYKCKVSNKALPAPIEKTISKAKGQPREPOQVYTLPPSREEMTKNOVSLTCLV |
| | | KGFYPSDIAVEWESNGQPENNYKTTPPVLDSDGSFFLYSKLTVDKSRWQQGNVFSCS |
| | | VMHEALNHNYTQKSLSLSPGGGGPTGTGESKAPLMVAVKDAVRGSPAINVAVHVFRKA |
| | | ADDTWEPFASGKTSESGELHGLTTEEEFVEGIYKVEIDTKSYWKALGISPFHEHAEVVFTA |
| | | NDSGPRRYTIAALLSPYSYSTTAVVTNPKE |
| 32 | 655-341 Ab | ATGGAAACCCCAGCGCAGCTTCTCTTCCTCCTGCTACTCTGGCTCCCAGATACCA |
| | LC NA | CCGGAGAAATTGTGTTGACGCAGTCTCCAGGCACCCTGTCTTTGTCTCCAGGGGA |
| | (TTR | AAGAGCCACCCTCTCCTGCAGGGCCAGTCAGGGTATTAGTAGAAGCGAATTA |
| | construct) | GCCTGGTACCAGCAGAAACCTGGCCAGGCTCCCAGCCTCCTCATCTATGGTGCA |
| | | TCCAGCAGGGCCACTGGCATCCCAGACAGGTTCAGTGGCAGTGGGTCTGGGAC |
| | | AGACTTCACTCTCACCATCAGCAGACTGGAGCCTGAAGATTTTGCAGTGTATTAC |
| | | TGTCAACAATTTGGTAGTTCACCGTGGACGTTCGGCCAAGGGACCAAGGTGGA |
| | | AATCAAACGAACTGTGGCTGCACCATCTGTCTTCATCTTCCCGCCATCTGATGAG |
| | | CAGTTGAAATCTGGAACTGCTAGCGTTGTGTGCCTGCTGAATAACTTCTATCCCA |
| | | GAGAGGCCAAAGTACAGTGGAAGGTGGATAACGCCCTCCAATCGGGTAACTCCC |
| | | AGGAGAGTGTCACAGAGCAGGACAGCAAGGACAGCACCTACAGCCTCAGCAGC |
| | | ACCCTGACGCTGAGCAAAGCAGACTACGAGAAACACAAAGTCTACGCCTGCGAA |
| | | GTCACCCATCAGGGCCTGAGCTCGCCCGTCACAAAGAGCTTCAACAGGGGAGAG |
| | | TGT |
| 33 | 655-341 Ab | METPAQLLFLLLLWLPDTTGEIVLTQSPGTLSLSPGERATLSCRASQGISRSELAWYQ |
| | LC AA | QKPGQAPSLLIYGASSRATGIPDRFSGSGSGTDFTLTISRLEPEDFAVYYCQQFGSSP |
| | (TTR | WTFGQGTKVEIKRTVAAPSVFIFPPSDEQLKSGTASVYCLLNNFYPREAKVQWKVD |
| | construct) | NALQSGNSQESVTEQDSKDSTYSLSSTLTLSKADYEKHKVYACEYTHQGLSSPYTKS |
| | | FNRGEC |
| 34 | 655-341 Ab | ATGAAGCACCTGTGGTTCTTCCTCCTGCTGGTGGCAGCTCCCAGATGGGTCCTGT |
| | HC NA | CCCAGGTGCAGCTGCAGGAGTCGGGCCCAGGACTGGTGAAGCCTTCACAGACCC |
| | (TTR | TGTCCCTCACCTGCACTGTCTCTGGTGGCTCCATCAGCAGTGGTGATTACTTCTG |
| | construct) | GAGCTGGATCCGCCAGCTCCCAGGGAAGGGCCTGGAGTGGATTGGGCACATCC |
| | | ATAACAGTGGGACCACCTACTACAATCCGTCCCTCAAGAGTCGAGTTACCAT |
| | | ATCAGTAGACACGTCTAAGAAGCAGTTCTCCCTGAGGCTGAGTTCTGTGACTGCC |
| | | GCGGACACGGCCGTATATTACTGTGCGAGAGATCGAGGGGGTGACTACGCTTA |
| | | TGGTATGGACGTCTGGGGCCAAGGGACCACGGTCACCGTCTCCTCAGCCTCCAC |
| | | CAAGGGCCCATCCGTCTTCCCCCTGGCACCCTCCTCCAAGAGCACCTCTGGGGGC |
| | | ACAGCGGCCCTGGGCTGCCTGGTCAAGGACTACTTCCCCGAACCGGTGACGGTGT |
| | | CGTGGAACTCAGGCGCCCTGACCAGCGGCGTGCACACCTTCCCGGCTGTCCTACA |
| | | GTCCTCAGGACTCTACTCCCTCAGGAGCGTGGTGACCGTGCCCTCCAGCAGCTTG |
| | | GGCACCCAGACCTACATCTGCAACGTGAATCACAAGCCCAGCAACACCAAGGTG |
| | | GACAAGAGAGTTGAGCCCAAATCTTGTGACAAAACTCACACATGCCCACCGTGC |
| | | CCAGCACCTGAACTCCTGGGGGGACCGTCAGTCTTCCTCTTCCCCCCAAAACCCA |
| | | AGGACACCCTCATGATCTCCCGGACCCCTGAGGTCACATGCGTGGTGGTGGACGT |
| | | GAGCCACGAAGACCCTGAGGTCAAGTTCAACTGGTACGTGGACGGCGTGGAGGT |
| | | GCATAATGCCAAGACAAAGCCGCGGGAGGAGCAGTACAACAGCACGTACCGTGT |
| | | GGTCAGCGTCCTCACCGTCCTGCACCAGGACTGGCTGAATGGCAAGGAGTACAA |
| | | GTGCAAGGTCTCCAACAAAGCCCTCCCAGCCCCCATCGAGAAAACCATCTCCAA |
| | | AGCCAAAGGGCAGCCCCGAGAACCACAGGTGTACACCCTGCCCCCATCCCGGGA |
| | | GGAGATGACCAAGAACCAGGTCAGCCTGACCTGCCTGGTCAAAGGCTTCTATCC |
| | | CAGCGACATCGCCGTGGAGTGGGAGAGCAATGGGCAGCCGGAGAACAACTACA |
| | | AGACCACGCCTCCCGTGCTGGACTCCGACGGCTCCTTCTTCCTCTATAGCAAGCT |
| | | CACCGTGGACAAGAGCAGGTGGCAGCAGGGGAACGTCTTCTCATGCTCCGTGAT |
| | | GCATGAGGCTCTGCACAACCACTACACGCAGAAGAGCCTCTCCCTGTCTCCGGGT |
| | | GGAGGCGGTCCAACTGGTACTGGTGAATCTAAAGCTCCTCTTATGGTTGCAGTCG |
| | | ATGATGCTGTTCGTGGTTCCCCGGCAATTAATGTTGCTGTACATGTTTTCCGTAAA |
| | | GCTGCTGACGACACCTGGGAACCGTTCGCTAGCGGTAAAACCTCCGAATCCGGT |
| | | GAACTGCACGGTCTGACCACCGAAGAAGAATTCGTTGAAGGTATCTACAAAGTT |
| | | GAAATCGACACCAAATCCTACTGGAAAGCTTTGGGTATCTCCCCGTTCCACGAAC |
| | | ACGCTGAAGTTGTTTTCACCGCTAACGACTCCGGTCCGCGTCGTTACACGATCGC |
| | | TGCTCTGCTGTCCCCGTACTCCTACTCCACCACCGCTGTTGTTACCAACCCGAAAG |
| | | AA |
| 35 | 655-341 Ab | MKHLWFFLLLVAAPRWVLSQVQLQESGPGLVKPSQTLSLTCTVSGGSISSGDYFWS |
| | LC AA | WIRQLPGKGLEWIGHIHNSGTTYYNPSLKSRVTISVDTSKKQFSLRLSSVTAADTAV |
| | (TTR | YYCARDRGGDYAYGMDVWGQGTTVTVSSASTKGPSVFPLAPSSKSTSGGTAALGC |
| | construct) | LVKDYFPEPVTVSWNSGALTSGVHTFPAVLQSSGLYSLSSVVTVPSSSLGTQTYICNV |
| | | NHKPSNTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLTVLHQDWL |
| | | NGKEYKCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVFSCSVM |
| | | HEALHNHYTQKSLSLSPGGGGPTGTGESKAPLMVAVDDAVRGSPAINVAVHVFRKAAD |
| | | DTWEPFASGKTSESGELHGLTTEEEFVEGIYKVEIDTKSYWKALGISPFHEHAEVVFTAND |
| | | SGPRRYTIAALLSPYSYSTTAVVTNPKE |
| 36 | 655-341 | ATGGAAACCCCAGCGCAGCTTCTCTTCCTCCTGCTACTCTGGCTCCCAGATACCA |
| | Fab LC NA | CCGGAGAAATTGTGTTGACGCAGTCTCCAGGCACCCTGTCTTTGTCTCCAGGGGA |
| | (TTR | AAGAGCCACCCTCTCCTGCAGGGCCAGTCAGGGTATTAGTAGAAGCGAATTA |
| | construct) | GCCTGGTACCAGCAGAAACCTGGCCAGGCTCCCAGCCTCCTCATCTATGGTGCA |
| | | TCCAGCAGGGCCACTGGCATCCCAGACAGGTTCAGTGGCAGTGGGTCTGGGAC |
| | | AGACTTCACTCTCACCATCAGCAGACTGGAGCCTGAAGATTTTGCAGTGTATTAC |
| | | TGTCAACAATTTGGTAGTTCACCGTGGACGTTCGGCCAAGGGACCAAGGTGGA |
| | | AATCAAACGAACTGTGGCTGCACCATCTGTCTTCATCTTCCCGCCATCTGATGAG |
| | | CAGTTGAAATCTGGAACTGCTAGCGTTGTGTGCCTGCTGAATAACTTCTATCCCA |
| | | GAGAGGCCAAAGTACAGTGGAAGGTGGATAACGCCCTCCAATCGGGTAACTCCC |
| | | AGGAGAGTGTCACAGAGCAGGACAGCAAGGACAGCACCTACAGCCTCAGCAGC |
| | | ACCCTGACGCTGAGCAAAGCAGACTACGAGAAACACAAAGTCTACGCCTGCGAA |
| | | GTCACCCATCAGGGCCTGAGCTCGCCCGTCACAAAGAGCTTCAACAGGGGAGAG |
| | | TGT |
| 37 | 655-341 | METPAQLLFLLLLWLPDTTGEIVLTQSPGTLSLSPGERATLSCRASQGISRSELAWYQ |
| | Fab LC AA | QKPGQAPSLLIYGASSRATGIPDRFSGSGSGTDFTLTISRLEPEDFAVYYCQQFGSSP |
| | (TTR | WTFGQGTKVEIKRTVAAPSVFIFPPSDEQLKSGTASVVCLLNNFYPREAKVQWKVD |
| | construct) | NALQSGNSQESVTEQDSKDSTYSLSSTLTLSKADYEKHKVYACEVTHQGLSSPVTKS |
| | | FNRGEC |
| 38 | 655-341 | ATGAAGCACCTGTGGTTCTTCCTCCTGCTGGTGGCAGCTCCCAGATGGGTCCTGT |
| | Fab HC NA | CCCAGGTGCAGCTGCAGGAGTCGGGCCCAGGACTGGTGAAGCCTTCACAGACCC |
| | (TTR | TGTCCCTCACCTGCACTGTCTCTGGTGGCTCCATCAGCAGTGGTGATTACTTCTG |
| | construct) | GAGCTGGATCCGCCAGCTCCCAGGGAAGGGCCTGGAGTGGATTGGGCACATCC |
| | | ATAACAGTGGGACCACCTACTACAATCCGTCCCTCAAGAGTCGAGTTACCAT |
| | | ATCAGTAGACACGTCTAAGAAGCAGTTCTCCCTGAGGCTGAGTTCTGTGACTGCC |
| | | GCGGACACGGCCGTATATTACTGTGCGAGAGATCGAGGGGGTGACTACGCTTA |
| | | TGGTATGGACGTCTGGGGCCAAGGGACCACGGTCACCGTCTCCTCAGCCTCCAC |
| | | CAAGGGCCCATCCGTCTTCCCCCTGGCACCCTCCTCCAAGAGCACCTCTGGGGGC |
| | | ACAGCGGCCCTGGGCTGCCTGGTCAAGGACTACTTCCCCGAACCGGTGACGGTGT |
| | | CGTGGAACTCAGGCGCCCTGACCAGCGGCGTGCACACCTTCCCGGCTGTCCTACA |
| | | GTCCTCAGGACTCTACTCCCTCAGCAGCGTGGTGACCGTGCCCTCCAGCAGCTTG |
| | | GGCACCCAGACCTACATCTGCAACGTGAATCACAAGCCCAGCAACACCAAGGTG |
| | | GACAAGAGAGTTCAGCCCAAATCTTGTGGAGGAGGTCCAACTGGTACTGGTGAA |
| | | TCTAAAGCTCCTCTTATGGTTGCAGTCGATGATGCTGTTCGTGGTTCCCCGGCAAT |
| | | TAATGTTGCTGTACATGTTTTCCGTAAAGCTGCTGACGACACCTGGGAACCGTTC |
| | | GCTAGCGGTAAAACCTCCGAATCCGGTGAACTGCACGGTCTGACCACCGAAGAA |
| | | GAATTCGTTGAAGGTATCTACAAAGTTGAAATCGACACCAAATCCTACTGGAAA |
| | | GCTTTGGGTATCTCCCCGTTCCACGAACACGCTGAAGTTGTTTTCACCGCTAACG |
| | | ACTCCGGTTTCCGCGTCGTTACACGATCGCTGCTCTGCTGTCCCCGTACTCCTACTCC |
| | | ACCACCGCTGTTGTTACCAACCCGAAAGAAGGCGGTCACCATCACCATCACCAC |
| 39 | 655-341 | MKHLWFFLLLVAAPRWVLSQVQLQESGPGLVKPSQTLSLTCTVSGGSISSGDYFWS |
| | Fab HC AA | WIRQLPGKGLEWIGHIHNSGTTYYNPSLKSRVTISVDTSKKQFSLRLSSVTAADTAV |
| | (TTR | YYCARDRGGDYAYGMDVWGQGTTVTVSSASTKGPSVFPLAPSSKSTSGGTAALGC |
| | construct) | LVKDYFPEPVTVSWNSGALTSGVHTFPAVLQSSGLYSLSSVVTVPSSSLGTQTYICNV |
| | | NHKPSNTKVDKRVEPKSCGGGGPTGTGESKAPLMVAVDDAVRGSPAINVAVHVFRKAAD |
| | | DTWEPFASGKTSESGELHGLTTEEFVEGIYKVEIKTKSYWKALGISPFHEHAEVVFTAND |
| | | SGPRRYTIAALLSPYSYSTTAVVTNPKEGGHHHHHH |
| 40 | DNP 3B1 | ATGGACATGAGGGTGCCCGCTCAGCTCCTGGGGCTCCTGCTGCTGTGGCTGAGAG |
| | Fab LC NA | GTGCGCGCTGTGACATCCAGATGACCCAGTCTCCATCCTCCCTGTCTGCATCTGA |
| | (TTR | AGGAGACAGAGTCACCATCACTTGCCGGGCAAGTCAGGGCATTAGAAATGATT |
| | construct) | TAGGCTGGTATCAGCAGAAACCAGGGAAAGCCCCTAAGCGCCTGATCTATGCTG |
| | | CATCCAGTTTGCAAAGTGGGGTCCCATTAAGGTTCAGCGGCAGTGGATCTGGGA |
| | | CAGAATTCACTCTCACAATCAGCAGCCTGCAGCCTGAAGATTTTGCAACTTATTA |
| | | CTGTCTACAGTATAATAGTTACCCGTGGACGTTTCGGCCAAGGGACCAAGGTGG |
| | | AAATCAAACGTACGGTGGCTGCACCATCTGTCTTCATCTTCCCGCCATCTGATGA |
| | | GCAGTTGAAATCTGGAACTGCCTCTGTTGTGTGCCTGCTGAATAACTTCTATCCC |
| | | AGAGAGGCCAAAGTACAGTGGAAGGTGGATAACGCCCTCCAATCGGGTAACTCC |
| | | CAGGAGAGTGTCACAGAGCAGGACAGCAAGGACAGCACCTACAGCCTCAGCAG |
| | | CACCCTGACGCTGAGCAAAGCAGACTACGAGAAACACAAAGTCTACGCCTGCGA |
| | | AGTCACCCATCAGGGCCTGAGCTCGCCCGTCACAAAGAGCTTCAACAGGGGAGA |
| | | GTGT |
| 41 | DNP 3B1 | MDMRVPAQLLGLLLLWLRGARCDIQMTQSPSSLSASEGDRVTITCRASQGIRNDLG |
| | Fab LC AA | WVQQKPGKAPKRLIVAASSLQSGVPLRFSGSGSGTEFTLTISSLQPEDFATYYCLQYN |
| | (TTR | SVPWTFGQGTKVEIKRTVAAPSVFIFPPSDEQLKSGTASVVCLLNNFYPREAKVQWK |
| | construct) | VDNALQSGNSQESVTEQDSKDSTYSLSSTLTLSKADYEKHKVYACEVTHQGLSSPVT |
| | | KSFNRGEC |
| 42 | DNP 3B1 | ATGGACATGAGGGTGCCCGCTCAGCTCCTGGGGCTCCTGCTGCTGTGGCTGAGAG |
| | Fab HC NA | GTGCGCGCTGTCAGGTGCAGCTGCAGGAGTCGGGCCCAGGACTGGTTAAGCCTT |
| | (TTR | CGGAGACCCTGTCCCTCACCTGCACTGTCTCTGGTGGCTCCATCAGTAGTTACTA |
| | construct) | CTGGAGCTGGATCCGGCAGCCCCCAGGGAAGGGACTGGAGTGGATTGGGTATA |
| | | TCTATTACAGTGGGAACACCAACTCCAACCCCTCCCTCAAGAGTCGAGTCAC |
| | | CATATCAGTAGACACGTCCAAGAACCAGTTCTCCCTGAAGCTGAGCTCTGTGACC |
| | | GCTGCGGACACGGCCGTGTATTACTGTGCGAGAACCTACTATGATAGTAGTGG |
| | | TTACTACTACCGTGCTTTTGATATCTGGGGCCAAGGGACAATGGTCACCGTCTC |
| | | TAGTGCCTCCACCAAGGGCCCATCGGTCTTCCCCCTGGCACCCTCCTCCAAGAGC |
| | | ACCTCTGGGGGCACAGCGGCCCTGGGCTGCCTGGTCAAGGACTACTTCCCCGAAC |
| | | CGGTGACGGTGTCGTGGAACTCAGGCGCCCTGACCAGCGGCGTGCACACCTTCCC |
| | | GGCTGTCCTACAGTCCTCAGGACTCTACTCCCTCAGCAGCGTGGTGACCGTGCCC |
| | | TCCAGCAGCTTGGGCACCCAGACCTACATCTGCAACGTGAATCACAAGCCCAGC |
| | | AACACCAAGGTGGACAAGAAAGTTGAGCCCAAATCTTGTGGAGGAGGTCCAACT |
| | | GGTACTGGTGAATCTAAAGCTCCTCTTATGGTTGCAGTCAAAGATGCTGTTCGTG |
| | | GTTCCCCGGCAATTAATGTTGCTGTACATGTTTTCCGTAAAGCTGCTGACGACAC |
| | | CTGGGAACCGTTCGCTAGCGGTAAAACCTCCGAATCCGGTGAACTGCACGGTCTG |
| | | ACCACCGAAGAAGAATTCGTTGAAGGTATCTAGAAAGTTGAAATGGACACCAAA |
| | | TCCTACTGGAAAGCTTTGGGTATCTCCCCGTTCCACGAACACGCTGAAGTTGTTTT |
| | | CACCGCTAACGACTCCGGTCCGCGTCGTTACACGATCGCTGCTCTGCTGTCCCCG |
| | | TACTCCTACTCCACCACCGCTGTTGTTACCAACCCGAAAGAAGGCGGTCACCATC |
| | | ACCATCACCAC |
| 43 | DNP 3B1 | MWMRVPAQLLGLLLLWLRGARCQVQLQESGPGLVKPSETLSLTCTVSGGSISSYYW |
| | Fab HC AA | SWIRQPPGKGLEWIGYIYYSGNTNSNPSLKSRVTISVDTSKNQFSLKLSSVTAADTAV |
| | (TTR | YYCARTYYDSSGYYYRAFDIWGQGTMVTVSSASTKGPSVFPLAPSSKSTSGGTAAL |
| | construct) | GCLVKDYFPEPVTVSWNSGALTSGVHTFPAVLQSSGLYSLSSVVTVPSSSLGTQTYIC |
| | | NVNHKPSNTKVDKKVEPKSCGGGGPTGTGESKAPLMVAVKDAVRGSPAINVAVHVFRKA |
| | | ADDTWEPFASGKTSESGELHGLTTEEEFVEGIYKVEIDTKSYWKALGISPFHEHAEVVFTA |
| | | NDSGPRRYTIAALLSPYSYSTTAVVTNPKEGGHHHHHH |
| 44 | Human | GGTCCAACTGGTACCGGTGAATCCAAGTGTCCTCTGATGGTCAAAGTTCTAGATG |
| | TTR NA | CTGTCCGAGGCAGTCCTGCCATCAATGTGGCCGTGCATGTGTTCAGAAAGGCTGC |
| | | TGATGACACCTGGGAGCCATTTGCCTCTGGGAAAACCAGTGAGTCTGGAGAGCT |
| | | GCATGGGCTCACAACTGAGGAGGAATTTGTAGAAGGGATATACAAAGTGGAAAT |
| | | AGACACCAAATCTTACTGGAAGGCACTTGGCATCTCCCCATTCCATGAGCATGCA |
| | | GAGGTGGTATTCACAGCCAACGACTCCGGCCCCCGCCGCTACACCATTGCCGCCC |
| | | TGCTGAGCCCCTACTCCTATTCCACCACGGCTGTCGTCACCAATCCCAAGGAA |

Underline = Signal Peptide (The signal peptide may be removed from any amino acid or nucleic acid sequence disclosed herein. Accordingly, the sequence with and without the signal peptide is specifically contemplated for each amino acid and nucleic acid sequence recited herein).
Bold = CDRs
Bold + Underline = linker
Italics = TTR portion of fusion protein

1. A transthyretin (TTR) protein complex, wherein said TTR protein complex comprises TTR subunits A, B, C, and D;
wherein TTR subunits A and B dimerize to form TTR dimer AB;
wherein TTR subunits C and D dimerize to form TTR dimer CD;
wherein TTR dimers AB and CD further dimerize to form TTR tetramer ABCD; and
wherein each of A, B, C, and D comprise the amino acid sequence of SEQ ID NO: 1, except that at least one amino acid in the interface between TTR dimer AB and TTR dimer CD is mutated such that the formation of an ABCD tetramer is favored over the formation of an ABAB tetramer or a CDCD tetramer.

2. The TTR protein complex according to claim 1, wherein each of A, B, C, and D comprise the amino acid sequence of SEQ ID NO: 1 with the following mutations: C10A, K15A, or both a C10A and a K15A mutation.

3. The TTR protein complex according to claim 1, wherein both A and B, both C and D, or all four of A, B, C, and D comprise a mutation at one or more amino acids positions selected from the list comprising: 6, 7, 8, 9, 10, 13, 15, 17, 19, 20, 21, 22, 23, 24, 26, 50, 51, 52, 53, 54, 56, 57, 60, 61, 62, 63, 78, 82, 83, 84, 85, 100, 101, 102, 103, 104, 106, 108, 110, 112, 113, 114, 115, 117, 119, 121, 123, 124, 125, 126, and 127 of SEQ ID NO: 1.

4. (canceled)

5. (canceled)

6. (canceled)

7. The TTR protein complex according to claim 3, wherein A and B comprise a mutation at one or more amino acids positions selected from the list comprising: 15, 17, 20, 21, 22, 23, 24, 51, 52, 84, 106, 108, 112, 114, 115, 119, 121, and 123 of SEQ ID NO: 1,
wherein said amino acid is mutated to an aspartate or glutamate.

8. The TTR protein complex according to claim 3, wherein C and D comprise a mutation at one or more amino acids positions selected from the list comprising: 15, 17, 20, 21, 22, 23, 24, 51, 52, 84, 106, 108, 112, 114, 115, 119, 121, and 123 of SEQ ID NO: 1,
wherein said amino acid is mutated to an arginine, lysine, or histidine.

9. The TTR protein complex according to claim 1 wherein:
A and B comprise a mutation at one or more amino acids positions selected from the list comprising: 15, 17, 20, 21, 22, 23, 24, 51, 52, 84, 106, 108, 112, 114, 115, 119, 121, and 123 of SEQ ID NO: 1, wherein said amino acid is mutated to an aspartate or glutamate; and
C and D comprises a mutation at one or more amino acids positions selected from the list comprising: 15, 17, 20, 21, 22, 23, 24, 51, 52, 84, 106, 108, 112, 114, 115, 119, 121, and 123 of SEQ ID NO: 1, wherein said amino acid is mutated to an arginine, lysine, or histidine.

10. The TTR protein complex according to claim 9, wherein A and B comprise at least one mutation in SEQ ID NO: 1, wherein said mutation is selected from the list comprising: K15D, L17D, V20D, R21D, G22D, S23D, P24D, S52D, I84D, T106D, A108D, S112D, Y114D, S115D, T119D, V121D, S123D, K15E, L17E, V20E, R21E, G22E, S23E, P24E, D51E, S52E, I84E, T106E, A108E, S112E, Y114E, S115E, T119E, V121E, and S123E.

11. The TTR protein complex according to claim 10, wherein A and B comprise at least one mutation in SEQ ID NO: 1, wherein said mutation is selected from the list comprising: L17D, L17E, V20D, V20E, G22D, G22E, S112D, S112E, T119D, T119E, V121D, and V121E.

12. The TTR protein complex according to claim 9, wherein C and D comprise at least one mutation in SEQ ID NO: 1, wherein said mutation is selected from the list comprising: K15R, L17R, V20R, G22R, S23R, P24R, D51R, S52R, I84R, T106R, A108R, S112R, Y114R, S115R, T119R, V121R, S123R, L17K, V20K, R21K, G22K, S23K, P24K, D51K, S52K, I84K, T106K, A108K, S112K, Y114K, S115K, T119K, V121K, S123K, K15H, L17H, V20H, R21H, G22H, S23H, P24H, D51H, S52H, 184H, T106H, A108H, S112H, Y114H, S115H, T119H, V121H, and S123H.

13. The TTR protein complex according to claim 12, wherein C and D comprise at least one mutation in SEQ ID NO: 1, wherein said mutation is selected from the list comprising: L17R, L17K, L17H, V20R, V20K, V20H, G22R, G22K, G22H, S112R, S112K, S112H, T119R, T119K, T119H, V121R, V121K, and V121H.

14. The TTR protein complex according to claim 9, wherein both A and B, both C and D, or all four of A, B, C, and D independently comprise one said mutation.

15. The TTR protein complex according to claim 9, wherein both A and B, both C and D, or all four of A, B, C, and D independently comprise two said mutations.

16. The TTR protein complex according to claim 1, wherein each of A, B, C, and D comprise the amino acid sequence of SEQ ID NO: 1 with the following mutations:
both A and B comprise C10A/K15A/L17D, and both C and D comprise C10A/K15A/L17R;
both A and B comprise C10A/K15A/L17E, and both C and D comprise C10A/K15A/L17R;
both A and B comprise C10A/K15A/V20D, and both C and D comprise C10A/K15A/L17R;
both A and B comprise C10A/K15A/V20E, and both C and D comprise C10A/K15A/L17R;
both A and B comprise C10A/K15A/G22D, and both C and D comprise C10A/K15A/L17R;
both A and B comprise C10A/K15A/G22E, and both C and D comprise C10A/K15A/L17R;
both A and B comprise C10A/K15A/S112D, and both C and D comprise C10A/K15A/L17R;
both A and B comprise C10A/K15A/S112E, and both C and D comprise C10A/K15A/L17R;
both A and B comprise C10A/K15A/T19D, and both C and D comprise C10A/K15A/L17R;
both A and B comprise C10A/K15A/T119E, and both C and D comprise C10A/K15A/L17R;
both A and B comprise C10A/K15A/V121D, and both C and D comprise C10A/K15A/L17R;
both A and B comprise C10A/K15A/V121E, and both C and D comprise C10A/K15A/L17R;
both A and B comprise C10A/K15A/L17D, and both C and D comprise C10A/K15A/L17K;
both A and B comprise C10A/K15A/L17E, and both C and D comprise C10A/K15A/L17K;
both A and B comprise C10A/K15A/V20D, and both C and D comprise C10A/K15A/L17K;
both A and B comprise C10A/K15A/V20E, and both C and D comprise C10A/K15A/L17K;
both A and B comprise C10A/K15A/G22D, and both C and D comprise C10A/K15A/L17K;
both A and B comprise C10A/K15A/G22E, and both C and D comprise C10A/K15A/L17K;
both A and B comprise C10A/K15A/S112D, and both C and D comprise C10A/K15A/L17K;
both A and B comprise C10A/K15A/S112E, and both C and D comprise C10A/K15A/L17K;
both A and B comprise C10A/K15A/T19D, and both C and D comprise C10A/K15A/L17K;
both A and B comprise C10A/K15A/T119E, and both C and D comprise C10A/K15A/L17K;
both A and B comprise C10A/K15A/V121D, and both C and D comprise C10A/K15A/L17K;
both A and B comprise C10A/K15A/V121E, and both C and D comprise C10A/K15A/L17K;
both A and B comprise C10A/K15A/L17D, and both C and D comprise C10A/K15A/V20R;
both A and B comprise C10A/K15A/L17E, and both C and D comprise C10A/K15A/V20R;
both A and B comprise C10A/K15A/V20D, and both C and D comprise C10A/K15A/V20R;
both A and B comprise C10A/K15A/V20E, and both C and D comprise C10A/K15A/V20R;
both A and B comprise C10A/K15A/G22D, and both C and D comprise C10A/K15A/V20R;
both A and B comprise C10A/K15A/G22E, and both C and D comprise C10A/K15A/V20R;
both A and B comprise C10A/K15A/S112D, and both C and D comprise C10A/K15A/V20R;
both A and B comprise C10A/K15A/S112E, and both C and D comprise C10A/K15A/V20R;
both A and B comprise C10A/K15A/T119D, and both C and D comprise C10A/K15A/V20R;
both A and B comprise C10A/K15A/T119E, and both C and D comprise C10A/K15A/V20R;
both A and B comprise C10A/K15A/V121D, and both C and D comprise C10A/K15A/V20R;
both A and B comprise C10A/K15A/V121E, and both C and D comprise C10A/K15A/V20R;
both A and B comprise C10A/K15A/L17D, and both C and D comprise C10A/K15A/V20K;
both A and B comprise C10A/K15A/L17E, and both C and D comprise C10A/K15A/V20K;
both A and B comprise C10A/K15A/V20D, and both C and D comprise C10A/K15A/V20K;
both A and B comprise C10A/K15A/V20E, and both C and D comprise C10A/K15A/V20K;
both A and B comprise C10A/K15A/G22D, and both C and D comprise C10A/K15A/V20K;
both A and B comprise C10A/K15A/G22E, and both C and D comprise C10A/K15A/V20K;
both A and B comprise C10A/K15A/S112D, and both C and D comprise C10A/K15A/V20K;
both A and B comprise C10A/K15A/S112E, and both C and D comprise C10A/K15A/V20K;
both A and B comprise C10A/K15A/T119D, and both C and D comprise C10A/K15A/V20K;
both A and B comprise C10A/K15A/T119E, and both C and D comprise C10A/K15A/V20K;
both A and B comprise C10A/K15A/V121D, and both C and D comprise C10A/K15A/V20K;
both A and B comprise C10A/K15A/V121E, and both C and D comprise C10A/K15A/V20K;
both A and B comprise C10A/K15A/L17D, and both C and D comprise C10A/K15A/G22R;
both A and B comprise C10A/K15A/L17E, and both C and D comprise C10A/K15A/G22R;
both A and B comprise C10A/K15A/V20D, and both C and D comprise C10A/K15A/G22R;
both A and B comprise C10A/K15A/V20E, and both C and D comprise C10A/K15A/G22R;
both A and B comprise C10A/K15A/G22D, and both C and D comprise C10A/K15A/G22R;
both A and B comprise C10A/K15A/G22E, and both C and D comprise C10A/K15A/G22R;
both A and B comprise C10A/K15A/S112D, and both C and D comprise C10A/K15A/G22R;
both A and B comprise C10A/K15A/S112E, and both C and D comprise C10A/K15A/G22R;
both A and B comprise C10A/K15A/T119D, and both C and D comprise C10A/K15A/G22R;
both A and B comprise C10A/K15A/T119E, and both C and D comprise C10A/K15A/G22R;
both A and B comprise C10A/K15A/V121D, and both C and D comprise C10A/K15A/G22R;
both A and B comprise C10A/K15A/V121E, and both C and D comprise C10A/K15A/G22R;
both A and B comprise C10A/K15A/L17D, and both C and D comprise C10A/K15A/G22K;
both A and B comprise C10A/K15A/L17E, and both C and D comprise C10A/K15A/G22K;
both A and B comprise C10A/K15A/V20D, and both C and D comprise C10A/K15A/G22K;
both A and B comprise C10A/K15A/V20E, and both C and D comprise C10A/K15A/G22K;
both A and B comprise C10A/K15A/G22D, and both C and D comprise C10A/K15A/G22K;
both A and B comprise C10A/K15A/G22E, and both C and D comprise C10A/K15A/G22K;
both A and B comprise C10A/K15A/S112D, and both C and D comprise C10A/K15A/G22K;
both A and B comprise C10A/K15A/S112E, and both C and D comprise C10A/K15A/G22K;
both A and B comprise C10A/K15A/T119D, and both C and D comprise C10A/K15A/G22K;
both A and B comprise C10A/K15A/T119E, and both C and D comprise C10A/K15A/G22K;
both A and B comprise C10A/K15A/V121D, and both C and D comprise C10A/K15A/G22K;
both A and B comprise C10A/K15A/V121E, and both C and D comprise C10A/K15A/G22K;
both A and B comprise C10A/K15A/L17D, and both C and D comprise C10A/K15A/S112R;
both A and B comprise C10A/K15A/L17E, and both C and D comprise C10A/K15A/S112R;
both A and B comprise C10A/K15A/V20D, and both C and D comprise C10A/K15A/S112R;
both A and B comprise C10A/K15A/V20E, and both C and D comprise C10A/K15A/S112R;
both A and B comprise C10A/K15A/G22D, and both C and D comprise C10A/K15A/S112R;
both A and B comprise C10A/K15A/G22E, and both C and D comprise C10A/K15A/S112R;
both A and B comprise C10A/K15A/S112D, and both C and D comprise C10A/K15A/S112R;
both A and B comprise C10A/K15A/S112E, and both C and D comprise C10A/K15A/S112R;
both A and B comprise C10A/K15A/T119D, and both C and D comprise C10A/K15A/S112R;
both A and B comprise C10A/K15A/T119E, and both C and D comprise C10A/K15A/S112R;
both A and B comprise C10A/K15A/V121D, and both C and D comprise C10A/K15A/S112R;
both A and B comprise C10A/K15A/V121E, and both C and D comprise C10A/K15A/S112R;
both A and B comprise C10A/K15A/L17D, and both C and D comprise C10A/K15A/S112K;
both A and B comprise C10A/K15A/L17E, and both C and D comprise C10A/K15A/S112K;
both A and B comprise C10A/K15A/V20D, and both C and D comprise C10A/K15A/S112K;
both A and B comprise C10A/K15A/V20E, and both C and D comprise C10A/K15A/S112K;
both A and B comprise C10A/K15A/G22D, and both C and D comprise C10A/K15A/S112K;
both A and B comprise C10A/K15A/G22E, and both C and D comprise C10A/K15A/S112K;
both A and B comprise C10A/K15A/S112D, and both C and D comprise C10A/K15A/S112K;
both A and B comprise C10A/K15A/S112E, and both C and D comprise C10A/K15A/S112K;
both A and B comprise C10A/K15A/T119D, and both C and D comprise C10A/K15A/S112K;
both A and B comprise C10A/K15A/T119E, and both C and D comprise C10A/K15A/S112K;
both A and B comprise C10A/K15A/V121D, and both C and D comprise C10A/K15A/S112K;
both A and B comprise C10A/K15A/V121E, and both C and D comprise C10A/K15A/S112K;
both A and B comprise C10A/K15A/L17D, and both C and D comprise C10A/K15A/T119R;
both A and B comprise C10A/K15A/L17E, and both C and D comprise C10A/K15A/T119R;
both A and B comprise C10A/K15A/V20D, and both C and D comprise C10A/K15A/T119R;
both A and B comprise C10A/K15A/V20E, and both C and D comprise C10A/K15A/T119R;
both A and B comprise C10A/K15A/G22D, and both C and D comprise C10A/K15A/T119R;
both A and B comprise C10A/K15A/G22E, and both C and D comprise C10A/K15A/T119R;
both A and B comprise C10A/K15A/S112D, and both C and D comprise C10A/K15A/T119R;
both A and B comprise C10A/K15A/S112E, and both C and D comprise C10A/K15A/T119R;
both A and B comprise C10A/K15A/T119D, and both C and D comprise C10A/K15A/T119R;
both A and B comprise C10A/K15A/T119E, and both C and D comprise C10A/K15A/T119R;
both A and B comprise C10A/K15A/V121D, and both C and D comprise C10A/K15A/T119R;
both A and B comprise C10A/K15A/V121E, and both C and D comprise C10A/K15A/T119R;
both A and B comprise C10A/K15A/L17D, and both C and D comprise C10A/K15A/T119K;
both A and B comprise C10A/K15A/L17E, and both C and D comprise C10A/K15A/T119K;
both A and B comprise C10A/K15A/V20D, and both C and D comprise C10A/K15A/T119K;
both A and B comprise C10A/K15A/V20E, and both C and D comprise C10A/K15A/T119K;
both A and B comprise C10A/K15A/G22D, and both C and D comprise C10A/K15A/T119K;
both A and B comprise C10A/K15A/G22E, and both C and D comprise C10A/K15A/T119K;
both A and B comprise C10A/K15A/S112D, and both C and D comprise C10A/K15A/T119K;
both A and B comprise C10A/K15A/S112E, and both C and D comprise C10A/K15A/T119K;
both A and B comprise C10A/K15A/T119D, and both C and D comprise C10A/K15A/T119K;
both A and B comprise C10A/K15A/T119E, and both C and D comprise C10A/K15A/T119K;
both A and B comprise C10A/K15A/V121D, and both C and D comprise C10A/K15A/T119K;
both A and B comprise C10A/K15A/V121E, and both C and D comprise C10A/K15A/T119K;
both A and B comprise C10A/K15A/L17D, and both C and D comprise C10A/K15A/V121R;
both A and B comprise C10A/K15A/L17E, and both C and D comprise C10A/K15A/V121R;
both A and B comprise C10A/K15A/V20D, and both C and D comprise C10A/K15A/V121R;
both A and B comprise C10A/K15A/V20E, and both C and D comprise C10A/K15A/V121R;
both A and B comprise C10A/K15A/G22D, and both C and D comprise C10A/K15A/V121R;
both A and B comprise C10A/K15A/G22E, and both C and D comprise C10A/K15A/V121R;
both A and B comprise C10A/K15A/S112D, and both C and D comprise C10A/K15A/V121R;
both A and B comprise C10A/K15A/S112E, and both C and D comprise C10A/K15A/V121R;
both A and B comprise C10A/K15A/T119D, and both C and D comprise C10A/K15A/V121R;
both A and B comprise C10A/K15A/T119E, and both C and D comprise C10A/K15A/V121R;
both A and B comprise C10A/K15A/V121D, and both C and D comprise C10A/K15A/V121R;
both A and B comprise C10A/K15A/V121E, and both C and D comprise C10A/K15A/V121R;
both A and B comprise C10A/K15A/L17D, and both C and D comprise C10A/K15A/V121K;
both A and B comprise C10A/K15A/L17E, and both C and D comprise C10A/K15A/V121K;
both A and B comprise C10A/K15A/V20D, and both C and D comprise C10A/K15A/V121K;
both A and B comprise C10A/K15A/V20E, and both C and D comprise C10A/K15A/V121K;
both A and B comprise C10A/K15A/G22D, and both C and D comprise C10A/K15A/V121K;
both A and B comprise C10A/K15A/G22E, and both C and D comprise C10A/K15A/V121K;
both A and B comprise C10A/K15A/S112D, and both C and D comprise C10A/K15A/V121K;
both A and B comprise C10A/K15A/S112E, and both C and D comprise C10A/K15A/V121K;
both A and B comprise C10A/K15A/T119D, and both C and D comprise C10A/K15A/V121K;
both A and B comprise C10A/K15A/T119E, and both C and D comprise C10A/K15A/V121K;
both A and B comprise C10A/K15A/V121D, and both C and D comprise C10A/K15A/V121K; or
both A and B comprise C10A/K15A/V121E, and both C and D comprise C10A/K15A/V121K.

17. The TTR protein complex according to claim 1, wherein:
both A and B comprise C10A/K15A/L17D, and both C and D comprise C10A/K15A/V121R;
both A and B comprise C10A/K15A/L17D, and both C and D comprise C10A/K15A/V121K;
both A and B comprise C10A/K15A/L17E, and both C and D comprise C10A/K15A/V121R;
both A and B comprise C10A/K15A/V20D, and both C and D comprise C10A/K15A/V20R;
both A and B comprise C10A/K15A/V20D, and both C and D comprise C10A/K15A/V20K;
both A and B comprise C10A/K15A/V20E, and both C and D comprise C10A/K15A/V20R;
both A and B comprise C10A/K15A/V20E, and both C and D comprise C10A/K15A/V20K;
both A and B comprise C10A/K15A/T119D, and both C and D comprise C10A/K15A/L17R;
both A and B comprise C10A/K15A/T119D, and both C and D comprise C10A/K15A/L17K; or
both A and B comprise C10A/K15A/V121E, and both C and D comprise C10A/K15A/L17K.

18. The TTR protein complex according to claim 1, wherein each of A, B, C, and D comprise the amino acid sequence of SEQ ID NO: 1 with the following mutations:
both A and B comprise C10A/K15A/L17D/V20D, and both C and D comprise C10A/K15A/L17K/V20K;
both A and B comprise C10A/K15A/L17D/V20E, and both C and D comprise C10A/K15A/L17K/V20R;
both A and B comprise C10A/K15A/L17E/V20D, and both C and D comprise C10A/K15A/L17R/V20K;
both A and B comprise C10A/K15A/L17E/V20E, and both C and D comprise C10A/K15A/L17R/V20R;
both A and B comprise C10A/K15A/L17D/T119D, and both C and D comprise C10A/K15A/L17K/V121K;
both A and B comprise C10A/K15A/L17D/V121E, and both C and D comprise C10A/K15A/L17K/V121R;
both A and B comprise C10A/K15A/L17E/T119D, and both C and D comprise C10A/K15A/L17R/V121K;
both A and B comprise C10A/K15A/L17E/V121E, and both C and D comprise C10A/K15A/L17R/V121R;
both A and B comprise C10A/K15A/V20D/T119D, and both C and D comprise C10A/K15A/V20K/V121K;
both A and B comprise C10A/K15A/V20D/V121E, and both C and D comprise C10A/K15A/V20K/V121R;
both A and B comprise C10A/K15A/V20E/T119D, and both C and D comprise C10A/K15A/V20R/V121K; or
both A and B comprise C10A/K15A/V20E/V121E, and both C and D comprise C10A/K15A/V20R/V121R.

19. The TTR protein complex according to claim 1, wherein each of A, B, C, and D comprise the amino acid sequence of SEQ ID NO: 1 with the following mutations:
both A and B comprise C10A/K15A/V20E/T119D, and both C and D comprise C10A/K15A/V20R/V121K;
both A and B comprise C10A/K15A/L17D/T119D, and both C and D comprise C10A/K15A/L17K/V121K;
both A and B comprise C10A/K15A/L17E T119D, and both C and D comprise C10A/K15A/L17R/V121K;
both A and B comprise C10A/K15A/L17E/V20D, and both C and D comprise C10A/K15A/L17R/V20K;
both A and B comprise C10A/K15A/L17D/V20D, and both C and D comprise C10A/K15A/L17K/V20K; or
both A and B comprise C10A/K15A/L17E/V121E, and both C and D comprise C10A/K15A/L17R/V121R.

20. The TTR protein complex according to claim 1, wherein said TTR protein complex is attached to 1, 2, 3, 4, 5, 6, 7, or 8 antigen binding proteins or peptides.

21. (canceled)

22. The TTR protein complex according to claim 20, wherein said antigen binding protein or peptide is attached to the TTR protein complex at the C-terminus of a TTR subunit.

23. The TTR protein complex according to claim 20, wherein said antigen binding protein or peptide is attached to the TTR protein complex at the N-terminus of a TTR subunit.

24. The TTR protein complex according to claim 20, wherein said TTR protein complex is:
directly attached to 1, 2, 3, 4, 5, 6, 7, or 8 antigen binding proteins; or
attached to 1, 2, 3, 4, 5, 6, 7, or 8 antigen binding proteins via a linker.

25. (canceled)

26. (canceled)

27. (canceled)

28. (canceled)

29. (canceled)

30. The TTR protein complex according to claim 24, wherein said linker is G, GG, GGG, GGGG, GGGGG, GGGGGG, GGGGGGG, GGGGGGGG, GGGGGGGGG, or GGGGGGGGGG.

31. (canceled)

32. (canceled)

33. The TTR protein complex according to claim 24, wherein said linker is selected from the list comprising: GG, GGGG, GGGSGG, GGGSGGGG, and GGAGGGAGGG.

34. (canceled)

35. (canceled)

36. (canceled)

37. (canceled)

38. (canceled)

39. (canceled)

40. A pharmaceutical composition comprising a TTR protein complex according to claim 1.

41. A method of treating cancer using a TTR protein complex according to claim 1.

42. (canceled)

43. (canceled)

44. One or more isolated nucleic acid(s) encoding a TTR subunit, a TTR subunit fused or linked to an antigen binding protein or peptide (such as an antibody or Fab), or a TTR protein complex according to claim 1.

45. An expression vector comprising a nucleic acid of claim 44.

46. A recombinant host cell comprising a nucleic acid of claim 44 or a vector of claim 45.

47. (canceled)

48. A method of making a TTR protein complex according to claim 1, said method comprising:
a) culturing the recombinant host cell of claim 46 or 47; and
b) isolating the TTR protein complex from said culture.


SEQUENCE LISTING (44 sequences)

SEQ ID NO: 1 - protein, 127 aa, Artificial Sequence
  Human TTR AA
GPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 2 - protein, 127 aa, Artificial Sequence
  Murine TTR AA
GPAGAGESKCPLMVKVLDAVRGSPAVDVAVKVFKKTSEGSWEPFASGKTAESGELHGLTTDEKFVEGVYRVELDTKSYWKTLGISPFHEFADVVFTANDSGHRHYTIAALLSPYSYSTTAVVSNPQN

SEQ ID NO: 3 - DNA, 615 bp, Artificial Sequence
  Murine TTR NA
acagaagtccactcattcttggcaggatggcttctcatcgtctgctcctcctctgccttgctggactggtatttgtgtctgaggctggccctacgggcaccggtgaatccaagtgtcctctgatggtcaaagttctagatgctgtccgaggcagtcctgccatcaatgtggccgtgcatgtgttcagaaaggctgctgatgacacctgggagccatttgcctctgggaaaaccagtgagtctggagagctgcatgggctcacaactgaggaggaatttgtagaagggatatacaaagtggaaatagacaccaaatcttactggaaggcacttggcatctccccattccatgagcatgcagaggtggtattcacagccaacgactccggcccccgccgctacaccattgccgccctgctgagcccctactcctattccaccacggctgtcgtcaccaatcccaaggaatgagggacttctcctccagtggacctgaaggacgagggatgggatttcatgtaaccaagagtattccatttttactaaagcactgttttcacctcatatgctatgttagaagtccaggcagagacaataaaacattcctgtgaaaggc

SEQ ID NO: 4 - protein, 236 aa, Artificial Sequence
  DNP 3B1 Ab LC AA
MDMRVPAQLLGLLLLWLRGARCDIQMTQSPSSLSASEGDRVTITCRASQGIRNDLGWYQQKPGKAPKRLIYAASSLQSGVPLRFSGSGSGTEFTLTISSLQPEDFATYYCLQYNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 708 bp, Artificial Sequence
  DNP Ab LC NA
atggacatgagggtgcccgctcagctcctggggctcctgctgctgtggctgagaggtgcgcgctgtgacatccagatgacccagtctccatcctccctgtctgcatctgaaggagacagagtcaccatcacttgccgggcaagtcagggcattagaaatgatttaggctggtatcagcagaaaccagggaaagcccctaagcgcctgatctatgctgcatccagtttgcaaagtggggtcccattaaggttcagcggcagtggatctgggacagaattcactctcacaatcagcagcctgcagcctgaagattttgcaacttattactgtctacagtataatagttacccgtggacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 6 - protein, 236 aa, Artificial Sequence
  DNP 3B1 Ab LC AA w/CPM S230E (EU S176E)
MDMRVPAQLLGLLLLWLRGARCDIQMTQSPSSLSASEGDRVTITCRASQGIRNDLGWYQQKPGKAPKRLIYAASSLQSGVPLRFSGSGSGTEFTLTISSLQPEDFATYYCLQYNSYPWTFGQGTKVEIKRTVAAPSVFIFPPSDEQLKSGTASVVCLLNNFYPREAKVQWKVDNALQSGNSQESVTEQDSKDSTYSLESTLTLSKADYEKHKVYACEVTHQGLSSPVTKSFNRGEC

SEQ ID NO: 7 - DNA, 708 bp, Artificial Sequence
  DNP 3B1 Ab LC NA w/CPM S230E (EU S176E)
atggacatgagggtgcccgctcagctcctggggctcctgctgctgtggctgagaggtgcgcgctgtgacatccagatgacccagtctccatcctccctgtctgcatctgaaggagacagagtcaccatcacttgccgggcaagtcagggcattagaaatgatttaggctggtatcagcagaaaccagggaaagcccctaagcgcctgatctatgctgcatccagtttgcaaagtggggtcccattaaggttcagcggcagtggatctgggacagaattcactctcacaatcagcagcctgcagcctgaagattttgcaacttattactgtctacagtataatagttacccgtggacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcgaaagcaccctgacgctgagcaaagcagactacgagaaacacaaagtctacgcctgcgaagtcacccatcagggcctgagctcgcccgtcacaaagagcttcaacaggggagagtgt

SEQ ID NO: 8 - protein, 474 aa, Artificial Sequence
  DNP 3B1 Ab HC AA
MDMRVPAQLLGLLLLWLRGARCQVQLQESGPGLVKPSETLSLTCTVSGGSISSYYWSWIRQPPGKGLEWIGYIYYSGNTNSNPSLKSRVTISVDTSKNQFSLKLSSVTAADTAVYYCARTYYDSSGYYYRAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - DNA, 1422 bp, Artificial Sequence
  DNP 3B1 Ab HC NA
atggacatgagggtgcccgctcagctcctggggctcctgctgctgtggctgagaggtgcgcgctgtcaggtgcagctgcaggagtcgggcccaggactggttaagccttcggagaccctgtccctcacctgcactgtctctggtggctccatcagtagttactactggagctggatccggcagcccccagggaagggactggagtggattgggtatatctattacagtgggaacaccaactccaacccctccctcaagagtcgagtcaccatatcagtagacacgtccaagaaccagttctccctgaagctgagctctgtgaccgctgcggacacggccgtgtattactgtgcgagaacctactatgatagtagtggttactactaccgtgcttttgatatctggggccaagggacaatggtcaccgtctctagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 10 - protein, 474 aa, Artificial Sequence
  DNP Ab HC AA w/CPM S230K (EU S183K)
MDMRVPAQLLGLLLLWLRGARCQVQLQESGPGLVKPSETLSLTCTVSGGSISSYYWSWIRQPPGKGLEWIGYIYYSGNTNSNPSLKSRVTISVDTSKNQFSLKLSSVTAADTAVYYCARTYYDSSGYYYRAFDIWGQGTMVTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - DNA, 1422 bp, Artificial Sequence
  DNP Ab HC NA w/CPM S230K (EU S183K)
atggacatgagggtgcccgctcagctcctggggctcctgctgctgtggctgagaggtgcgcgctgtcaggtgcagctgcaggagtcgggcccaggactggttaagccttcggagaccctgtccctcacctgcactgtctctggtggctccatcagtagttactactggagctggatccggcagcccccagggaagggactggagtggattgggtatatctattacagtgggaacaccaactccaacccctccctcaagagtcgagtcaccatatcagtagacacgtccaagaaccagttctccctgaagctgagctctgtgaccgctgcggacacggccgtgtattactgtgcgagaacctactatgatagtagtggttactactaccgtgcttttgatatctggggccaagggacaatggtcaccgtctctagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcaagagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 12 - protein, 248 aa, Artificial Sequence
  DNP Fab HC AA
MDMRVPAQLLGLLLLWLRGARCQVQLQESGPGLVKPSETLSLTCTVSGGSISSYYWSWIRQPPGKGLEWIGYIYYSGNTNSNPSLKSRVTISVDTSKNQFSLKLSSVTAADTAVYYCARTYYDSSGYYYRAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 13 - DNA, 744 bp, Artificial Sequence
  DNP Fab HC AA
atggacatgagggtgcccgctcagctcctggggctcctgctgctgtggctgagaggtgcgcgctgtcaggtgcagctgcaggagtcgggcccaggactggttaagccttcggagaccctgtccctcacctgcactgtctctggtggctccatcagtagttactactggagctggatccggcagcccccagggaagggactggagtggattgggtatatctattacagtgggaacaccaactccaacccctccctcaagagtcgagtcaccatatcagtagacacgtccaagaaccagttctccctgaagctgagctctgtgaccgctgcggacacggccgtgtattactgtgcgagaacctactatgatagtagtggttactactaccgtgcttttgatatctggggccaagggacaatggtcaccgtctctagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgt

SEQ ID NO: 14 - protein, 248 aa, Artificial Sequence
  DNP Fab HC AA w/CPM S230K (EU S183K)
MDMRVPAQLLGLLLLWLRGARCQVQLQESGPGLVKPSETLSLTCTVSGGSISSYYWSWIRQPPGKGLEWIGYIYYSGNTNSNPSLKSRVTISVDTSKNQFSLKLSSVTAADTAVYYCARTYYDSSGYYYRAFDIWGQGTMVTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKKVEPKSC

SEQ ID NO: 15 - DNA, 744 bp, Artificial Sequence
  DNP Fab HC NA w/CPM S230K (EU S183K)
atggacatgagggtgcccgctcagctcctggggctcctgctgctgtggctgagaggtgcgcgctgtcaggtgcagctgcaggagtcgggcccaggactggttaagccttcggagaccctgtccctcacctgcactgtctctggtggctccatcagtagttactactggagctggatccggcagcccccagggaagggactggagtggattgggtatatctattacagtgggaacaccaactccaacccctccctcaagagtcgagtcaccatatcagtagacacgtccaagaaccagttctccctgaagctgagctctgtgaccgctgcggacacggccgtgtattactgtgcgagaacctactatgatagtagtggttactactaccgtgcttttgatatctggggccaagggacaatggtcaccgtctctagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcaagagcgtggtgaccgtgccctccagcagcttgggcacccagacctacatctgcaacgtgaatcacaagcccagcaacaccaaggtggacaagaaagttgagcccaaatcttgt

SEQ ID NO: 16 - protein, 235 aa, Artificial Sequence
  655-341 Ab LC AA
METPAQLLFLLLLWLPDTTGEIVLTQSPGTLSLSPGERATLSCRASQGISRSELAWYQQKPGQAPSLLIYGASSRATGIPDRFSGSGSGTDFTLTISRLEPEDFAVYYCQQFGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - DNA, 705 bp, Artificial Sequence
  655-341 Ab LC NA
atggaaaccccagcgcagcttctcttcctcctgctactctggctcccagataccaccggagaaattgtgttgacgcagtctccaggcaccctgtctttgtctccaggggaaagagccaccctctcctgcagggccagtcagggtattagtagaagcgaattagcctggtaccagcagaaacctggccaggctcccagcctcctcatctatggtgcatccagcagggccactggcatcccagacaggttcagtggcagtgggtctgggacagacttcactctcaccatcagcagactggagcctgaagattttgcagtgtattactgtcaacaatttggtagttcaccgtggacgttcggccaagggaccaaggtggaaatcaaacgaactgtggctgcaccatctgtcttcatcttcccgccatctgatgagcagttgaaatctggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 18 - protein, 235 aa, Artificial Sequence
  655-341 Ab LC AA w/CPM S230K (EU S176K)
METPAQLLFLLLLWLPDTTGEIVLTQSPGTLSLSPGERATLSCRASQGISRSELAWYQQKPGQAPSLLIYGASSRATGIPDRFSGSGSGTDFTLTISRLEPEDFAVYYCQQFGSSPWTFGQGTKVEIKRTVAAPSVFIFPPSDEQLKSGTASVVCLLNNFYPREAKVQWKVDNALQSGNSQESVTEQDSKDSTYSLKSTLTLSKADYEKHKVYACEVTHQGLSSPVTKSFNRGEC

SEQ ID NO: 19 - DNA, 705 bp, Artificial Sequence
  655-341 Ab LC NA w/CPM S230K (EU S176K)
atggaaaccccagcgcagcttctcttcctcctgctactctggctcccagataccaccggagaaattgtgttgacgcagtctccaggcaccctgtctttgtctccaggggaaagagccaccctctcctgcagggccagtcagggtattagtagaagcgaattagcctggtaccagcagaaacctggccaggctcccagcctcctcatctatggtgcatccagcagggccactggcatcccagacaggttcagtggcagtgggtctgggacagacttcactctcaccatcagcagactggagcctgaagattttgcagtgtattactgtcaacaatttggtagttcaccgtggacgttcggccaagggaccaaggtggaaatcaaacgaactgtggctgcaccatctgtcttcatcttcccgccatctgatgagcagttgaaatctggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcaagagcaccctgacgctgagcaaagcagactacgagaaacacaaagtctacgcctgcgaagtcacccatcagggcctgagctcgcccgtcacaaagagcttcaacaggggagagtgt

SEQ ID NO: 20 - protein, 470 aa, Artificial Sequence
  655-341 Ab HC AA
MKHLWFFLLLVAAPRWVLSQVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYAYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - DNA, 1410 bp, Artificial Sequence
  655-341 Ab HC NA
atgaagcacctgtggttcttcctcctgctggtggcagctcccagatgggtcctgtcccaggtgcagctgcaggagtcgggcccaggactggtgaagccttcacagaccctgtccctcacctgcactgtctctggtggctccatcagcagtggtgattacttctggagctggatccgccagctcccagggaagggcctggagtggattgggcacatccataacagtgggaccacctactacaatccgtccctcaagagtcgagttaccatatcagtagacacgtctaagaagcagttctccctgaggctgagttctgtgactgccgcggacacggccgtatattactgtgcgagagatcgagggggtgactacgcttatggtatggacgtctggggccaagggaccacggtcaccgtctcctcagcctccaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 22 - protein, 470 aa, Artificial Sequence
  655-341 Ab HC AA w/CPM S230E (EU S183E)
MKHLWFFLLLVAAPRWVLSQVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYAYGMDVWGQGTTVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - DNA, 1410 bp, Artificial Sequence
  655-341 Ab HC NA w/CPM S230E (EU S183E)
atgaagcacctgtggttcttcctcctgctggtggcagctcccagatgggtcctgtcccaggtgcagctgcaggagtcgggcccaggactggtgaagccttcacagaccctgtccctcacctgcactgtctctggtggctccatcagcagtggtgattacttctggagctggatccgccagctcccagggaagggcctggagtggattgggcacatccataacagtgggaccacctactacaatccgtccctcaagagtcgagttaccatatcagtagacacgtctaagaagcagttctccctgaggctgagttctgtgactgccgcggacacggccgtatattactgtgcgagagatcgagggggtgactacgcttatggtatggacgtctggggccaagggaccacggtcaccgtctcctcagcctccaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcgagagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 24 - protein, 244 aa, Artificial Sequence
  655-341 Fab HC AA
MKHLWFFLLLVAAPRWVLSQVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYAYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 25 - DNA, 732 bp, Artificial Sequence
  655-341 Fab HC NA
atgaagcacctgtggttcttcctcctgctggtggcagctcccagatgggtcctgtcccaggtgcagctgcaggagtcgggcccaggactggtgaagccttcacagaccctgtccctcacctgcactgtctctggtggctccatcagcagtggtgattacttctggagctggatccgccagctcccagggaagggcctggagtggattgggcacatccataacagtgggaccacctactacaatccgtccctcaagagtcgagttaccatatcagtagacacgtctaagaagcagttctccctgaggctgagttctgtgactgccgcggacacggccgtatattactgtgcgagagatcgagggggtgactacgcttatggtatggacgtctggggccaagggaccacggtcaccgtctcctcagcctccaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgt

SEQ ID NO: 26 - protein, 244 aa, Artificial Sequence
  655-341 Fab HC AA w/CPM S230E (EU S183E)
MKHLWFFLLLVAAPRWVLSQVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYAYGMDVWGQGTTVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKRVEPKSC

SEQ ID NO: 27 - DNA, 732 bp, Artificial Sequence
  655-341 Fab HC NA w/CPM S230E (EU S183E)
atgaagcacctgtggttcttcctcctgctggtggcagctcccagatgggtcctgtcccaggtgcagctgcaggagtcgggcccaggactggtgaagccttcacagaccctgtccctcacctgcactgtctctggtggctccatcagcagtggtgattacttctggagctggatccgccagctcccagggaagggcctggagtggattgggcacatccataacagtgggaccacctactacaatccgtccctcaagagtcgagttaccatatcagtagacacgtctaagaagcagttctccctgaggctgagttctgtgactgccgcggacacggccgtatattactgtgcgagagatcgagggggtgactacgcttatggtatggacgtctggggccaagggaccacggtcaccgtctcctcagcctccaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcgagagcgtggtgaccgtgccctccagcagcttgggcacccagacctacatctgcaacgtgaatcacaagcccagcaacaccaaggtggacaagagagttgagcccaaatcttgt

SEQ ID NO: 28 - DNA, 708 bp, Artificial Sequence; identical to SEQ ID NO: 5
  DNP Ab LC NA (TTR construct)

SEQ ID NO: 29 - protein, 236 aa, Artificial Sequence; identical to SEQ ID NO: 4
  DNP Ab LC AA (TTR construct)

SEQ ID NO: 30 - DNA, 1809 bp, Artificial Sequence
  DNP Ab HC NA (TTR construct)
atggacatgagggtgcccgctcagctcctggggctcctgctgctgtggctgagaggtgcgcgctgtcaggtgcagctgcaggagtcgggcccaggactggttaagccttcggagaccctgtccctcacctgcactgtctctggtggctccatcagtagttactactggagctggatccggcagcccccagggaagggactggagtggattgggtatatctattacagtgggaacaccaactccaacccctccctcaagagtcgagtcaccatatcagtagacacgtccaagaaccagttctccctgaagctgagctctgtgaccgctgcggacacggccgtgtattactgtgcgagaacctactatgatagtagtggttactactaccgtgcttttgatatctggggccaagggacaatggtcaccgtctctagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggaggcggtccaactggtactggtgaatctaaagctcctcttatggttgcagtcaaagatgctgttcgtggttccccggcaattaatgttgctgtacatgttttccgtaaagctgctgacgacacctgggaaccgttcgctagcggtaaaacctccgaatccggtgaactgcacggtctgaccaccgaagaagaattcgttgaaggtatctacaaagttgaaatcgacaccaaatcctactggaaagctttgggtatctccccgttccacgaacacgctgaagttgttttcaccgctaacgactccggtccgcgtcgttacacgatcgctgctctgctgtccccgtactcctactccaccaccgctgttgttaccaacccgaaagaa

SEQ ID NO: 31 - protein, 603 aa, Artificial Sequence
  DNP Ab LC AA (TTR construct)
MDMRVPAQLLGLLLLWLRGARCQVQLQESGPGLVKPSETLSLTCTVSGGSISSYYWSWIRQPPGKGLEWIGYIYYSGNTNSNPSLKSRVTISVDTSKNQFSLKLSSVTAADTAVYYCARTYYDSSGYYYRAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGPTGTGESKAPLMVAVKDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 32 - DNA, 705 bp, Artificial Sequence; identical to SEQ ID NO: 17
  655-341 Ab LC NA (TTR construct)

SEQ ID NO: 33 - protein, 235 aa, Artificial Sequence; identical to SEQ ID NO: 16
  655-341 Ab LC AA (TTR construct)

SEQ ID NO: 34 - DNA, 1797 bp, Artificial Sequence
  655-341 Ab HC NA (TTR construct)
atgaagcacctgtggttcttcctcctgctggtggcagctcccagatgggtcctgtcccaggtgcagctgcaggagtcgggcccaggactggtgaagccttcacagaccctgtccctcacctgcactgtctctggtggctccatcagcagtggtgattacttctggagctggatccgccagctcccagggaagggcctggagtggattgggcacatccataacagtgggaccacctactacaatccgtccctcaagagtcgagttaccatatcagtagacacgtctaagaagcagttctccctgaggctgagttctgtgactgccgcggacacggccgtatattactgtgcgagagatcgagggggtgactacgcttatggtatggacgtctggggccaagggaccacggtcaccgtctcctcagcctccaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggaggcggtccaactggtactggtgaatctaaagctcctcttatggttgcagtcgatgatgctgttcgtggttccccggcaattaatgttgctgtacatgttttccgtaaagctgctgacgacacctgggaaccgttcgctagcggtaaaacctccgaatccggtgaactgcacggtctgaccaccgaagaagaattcgttgaaggtatctacaaagttgaaatcgacaccaaatcctactggaaagctttgggtatctccccgttccacgaacacgctgaagttgttttcaccgctaacgactccggtccgcgtcgttacacgatcgctgctctgctgtccccgtactcctactccaccaccgctgttgttaccaacccgaaagaa

SEQ ID NO: 35 - protein, 599 aa, Artificial Sequence
  655-341 Ab LC AA (TTR construct)
MKHLWFFLLLVAAPRWVLSQVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYAYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGPTGTGESKAPLMVAVDDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 36 - DNA, 705 bp, Artificial Sequence; identical to SEQ ID NO: 17
  655-341 Fab LC NA (TTR construct)

SEQ ID NO: 37 - protein, 235 aa, Artificial Sequence; identical to SEQ ID NO: 16
  655-341 Fab LC AA (TTR construct)

SEQ ID NO: 38 - DNA, 1143 bp, Artificial Sequence
  655-341 Fab HC NA (TTR construct)
atgaagcacctgtggttcttcctcctgctggtggcagctcccagatgggtcctgtcccaggtgcagctgcaggagtcgggcccaggactggtgaagccttcacagaccctgtccctcacctgcactgtctctggtggctccatcagcagtggtgattacttctggagctggatccgccagctcccagggaagggcctggagtggattgggcacatccataacagtgggaccacctactacaatccgtccctcaagagtcgagttaccatatcagtagacacgtctaagaagcagttctccctgaggctgagttctgtgactgccgcggacacggccgtatattactgtgcgagagatcgagggggtgactacgcttatggtatggacgtctggggccaagggaccacggtcaccgtctcctcagcctccaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtggaggaggtccaactggtactggtgaatctaaagctcctcttatggttgcagtcgatgatgctgttcgtggttccccggcaattaatgttgctgtacatgttttccgtaaagctgctgacgacacctgggaaccgttcgctagcggtaaaacctccgaatccggtgaactgcacggtctgaccaccgaagaagaattcgttgaaggtatctacaaagttgaaatcgacaccaaatcctactggaaagctttgggtatctccccgttccacgaacacgctgaagttgttttcaccgctaacgactccggtccgcgtcgttacacgatcgctgctctgctgtccccgtactcctactccaccaccgctgttgttaccaacccgaaagaaggcggtcaccatcaccatcaccac

SEQ ID NO: 39 - protein, 381 aa, Artificial Sequence
  655-341 Fab HC AA (TTR construct)
MKHLWFFLLLVAAPRWVLSQVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYAYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGPTGTGESKAPLMVAVDDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKEGGHHHHHH

SEQ ID NO: 40 - DNA, 708 bp, Artificial Sequence; identical to SEQ ID NO: 5
  DNP Fab LC NA (TTR construct)

SEQ ID NO: 41 - protein, 236 aa, Artificial Sequence; identical to SEQ ID NO: 4
  DNP Fab LC AA (TTR construct)

SEQ ID NO: 42 - protein, 1155 aa, Artificial Sequence
  DNP Fab HC NA (TTR construct)
ATGGACATGAGGGTGCCCGCTCAGCTCCTGGGGCTCCTGCTGCTGTGGCTGAGAGGTGCGCGCTGTCAGGTGCAGCTGCAGGAGTCGGGCCCAGGACTGGTTAAGCCTTCGGAGACCCTGTCCCTCACCTGCACTGTCTCTGGTGGCTCCATCAGTAGTTACTACTGGAGCTGGATCCGGCAGCCCCCAGGGAAGGGACTGGAGTGGATTGGGTATATCTATTACAGTGGGAACACCAACTCCAACCCCTCCCTCAAGAGTCGAGTCACCATATCAGTAGACACGTCCAAGAACCAGTTCTCCCTGAAGCTGAGCTCTGTGACCGCTGCGGACACGGCCGTGTATTACTGTGCGAGAACCTACTATGATAGTAGTGGTTACTACTACCGTGCTTTTGATATCTGGGGCCAAGGGACAATGGTCACCGTCTCTAGTGCCTCCACCAAGGGCCCATCGGTCTTCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAACTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGCTTGGGCACCCAGACCTACATCTGCAACGTGAATCACAAGCCCAGCAACACCAAGGTGGACAAGAAAGTTGAGCCCAAATCTTGTGGAGGAGGTCCAACTGGTACTGGTGAATCTAAAGCTCCTCTTATGGTTGCAGTCAAAGATGCTGTTCGTGGTTCCCCGGCAATTAATGTTGCTGTACATGTTTTCCGTAAAGCTGCTGACGACACCTGGGAACCGTTCGCTAGCGGTAAAACCTCCGAATCCGGTGAACTGCACGGTCTGACCACCGAAGAAGAATTCGTTGAAGGTATCTACAAAGTTGAAATCGACACCAAATCCTACTGGAAAGCTTTGGGTATCTCCCCGTTCCACGAACACGCTGAAGTTGTTTTCACCGCTAACGACTCCGGTCCGCGTCGTTACACGATCGCTGCTCTGCTGTCCCCGTACTCCTACTCCACCACCGCTGTTGTTACCAACCCGAAAGAAGGCGGTCACCATCACCATCACCAC

SEQ ID NO: 43 - protein, 385 aa, Artificial Sequence
  DNP Fab HC AA (TTR construct)
MDMRVPAQLLGLLLLWLRGARCQVQLQESGPGLVKPSETLSLTCTVSGGSISSYYWSWIRQPPGKGLEWIGYIYYSGNTNSNPSLKSRVTISVDTSKNQFSLKLSSVTAADTAVYYCARTYYDSSGYYYRAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGPTGTGESKAPLMVAVKDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKEGGHHHHHH

SEQ ID NO: 44 - DNA, 381 bp, Artificial Sequence
  Human TTR NA
ggtccaactggtaccggtgaatccaagtgtcctctgatggtcaaagttctagatgctgtccgaggcagtcctgccatcaatgtggccgtgcatgtgttcagaaaggctgctgatgacacctgggagccatttgcctctgggaaaaccagtgagtctggagagctgcatgggctcacaactgaggaggaatttgtagaagggatatacaaagtggaaatagacaccaaatcttactggaaggcacttggcatctccccattccatgagcatgcagaggtggtattcacagccaacgactccggcccccgccgctacaccattgccgccctgctgagcccctactcctattccaccacggctgtcgtcaccaatcccaaggaa